SEQUENCE LISTING (44 sequences)

SEQ ID NO: 1 - protein, 228 aa, Mycobacterium tuberculosis
MPEAKRPESKRRSPASRPGKAGDSVRGGRATKPSAKPSTPAPHASRKTTRTPHEHIVEPIKRAITESVEKRSEQRLGFTARRAAILAAVVCVLTLTIARPVRTYFAQRAEMEQLAATEAMLRRQIADLEEQQVKLADPAYIAAQARERLGFVMPGDIPFQVQLPSTPLAPPQPGSDAATATNNEPWYTALWHTIADDPHLPPAAPPAPEPGRPGPLPPASPNPEQPGG

SEQ ID NO: 2 - protein, 119 aa, Mycobacterium tuberculosis
MSKELTAKKRAALNRLKTVRGHLDGIVRMLESDAYCVDVMKQISAVQSSLERANRVMLHNHLETCFSTAVLDGHGQAAIEELIDAVKFTPALTGPHARLGGAAVGESATEEPMPDASNM

SEQ ID NO: 3 - protein, 765 aa, Mycobacterium tuberculosis
MAFPVISTYRVQMRGRSNGFGFTFADAENLLDYLDDLGVSHLYLSPILTAVGGSTHGYDVTDPTTVSPELGGSDGLARLSAAARSRGMGLIVDIVPSHVGVGKPEQNAWWWDVLKFGRSSAYAEFFDIDWELGDGRIILPLLGSDSDVANLRVDGDLLRLGDLALPVAPGSGDGTGPAVHDRQHYRLVGWRHGLCGYRRFFSITSLAGLRQEDRAVFDASHAEVARWFTEGLVDGVRVDHLDGLSDPSGYLAQLRELLGPNAWIVVEKILAVDEALEPTLPVDGSTGYDVLREIGGVLVDPQGESPLTALVESAGVDYQEMPAMLADLKVHAAVHTLASELRRLRRCIAAAAGADHPLLPAAVAALLRHIGRYRCDYPGQAAVLPCALAETHSTTPQLAPGLQLIAAAVARGGEPAVRLQQLCGAVSAKAVEDCMFYRDARLVSLNEVGGEPRRFGVGAAEFHHRAATRARLWPRSMTTLSTHDTKRGEDVRARIGVLSQVPWLWAKFIGHAQAIAPAPDAVTGQFLWQNVFGVWPVSGEVSAALRGRLHTYAEKAIREAAWHTSWHNPNRAFEDDVHGWLDLVLDGPLASELTGLVAHLNSHAESDALAAKLLALTVPGVPDVYQGSELWDDSLVDPDNRRPVDYGTRRVALKALQHPKIRVLAAALRLRRTHPESFLGGAYHPVFAAGPAADHVVAFRRGDDILVAVTRWTVRLQQTGWDHTVLPLPDGSWTDALTGFTASGHTPAVELFADLPVVLLVRDNA

SEQ ID NO: 4 - protein, 4151 aa, Mycobacterium tuberculosis
MVDQLQHATEALRKALVQVERLKRTNRALLERSSEPIAIVGMSCRFPGGVDSPEGLWQMVADARDVMSEFPTDRGWDLAGLFDPDPDVRHKSYARTGGFVDGVADFDPAFFGISPSEALAMDPQHRMLLELSWEALERAGIDPTGLRGSATGVFAGLIVGGYGMLAEEIEGYRLTGMTSSVASGRVAYVLGLEGPAVSVDTACSSSLVALHMAVGSLRSGECDLALAGGVTVNATPTVFVEFSRHRGLAPDGRCKPYAGRADGVGWSEGGGMLVLQRLSDARRLGHPVLAVVVGSAVNQDGASNGLTAPNGPSQQRVVRAALANAGLSAAEVDVVEGHGTGTTLGDPIEAQALLATYGQDRGEPGEPLWLGSVKSNMGHTQAAAGVAGVIKMVLAMRHELLPATLHVDVPSPHVDWSAGAVELLTAPRVWPAGARTRRAGVSSFGISGTNAHVIIEAVPVVPRREAGWAGPVVPWVVSAKSESALRGQAARLAAYVRGDDGLDVADVGWSLAGRSVFEHRAVVVGGDRDRLLAGLDELAGDQLGGSVVRGTATAAGKTVFVFPGQGSQWLGMGIELLDTAPAFAQQIDACAEAFAEFVDWSLVDVLRGAPGAPGLDRVDVVQPVLFAVMVSLAELWKSVAVHPDAVIGHSQGEIAAAYVAGALSLRDAARVVTLRSKLLAGLAGPGGMVSIACGADQARDLLAPFGDRVSIAVVNGPSAVVVSGEVGALEELIAVCSTKELRTRRIEVDYASHSVEVEAIRGPLAEALSGIEPRSTRTVFFSTVTGNRLDTAGLDADYWYRNVRQTVLFDQAVRNACEQGYRTFIESSPHPALITGVEETFAACTDGDSEAIVVPTLGRGDGGLHRFLLSAASAFVAGVAVNWRGTLDGAGYVELPTYAFDKRRFWLSAEGSGADVSGLGLGASEHPLLGAVVDLPASGGVVLTGRLSPNVQPWLADHAVSDVVLFPGTGFVELAIRAGDEVGCSVLDELTLAAPLLLPATGSVAVQVVVDAGRDSNSRGVSIFSRADAQAGWLLHAEGILRPGSVEPGADLSVWPPAGAVTVDVADGYERLATRGYRYGPAFRGLTAMWARGEEIFAEVRLPEAAGGVGGFGVHPALLDAVLHAVVIAGDPDELALPFAWQGVSLHATGASAVRARIAPAGPSAVSVELADGLGLPVLSVASMVARPVTERQLLAAVSGSGPDRLFEVIWSPASAATSPGPTPAYQIFESVAADQDPVAGSYVRSHQALAAVQSWLTDHESGVLVVATRGAMALPREDVADLAGAAVWGLVRSAQTEHPGRIVLVDSDAATDDAAIAMALATGEPQVVLRGGQVYTARVRGSRAADAILVPPGDGPWRLGLGSAGTFENLRLEPVPNADAPLGPGQVRVAMRAIAANFRDIMITLGMFTHDALLGGEGAGVVVEVGPGVTEFSVGDSVFGFFPDGSGTLVAGDVRLLLPMPADWSYAEAAAISAVFTTAYYAFIHLADVQPGQRVLIHAGTGGVGMAAVQLARHLGLEVFATASKGKWDTLRAMGFDDDHISDSRSLEFEDKFRAATGGRGFDVVLDSLAGEFVDASLRLVAPGGVFLEMGKTDIRDPGVIAQQYPGVRYRAFDLFEPGRPRMHQYMLELATLFGDGVLRPLPVTTFDVRRAPAALRYLSQARHTGKVVMLMPGSWAAGTVLITGGTGMAGSAVARHVVARHGVRNLVLVSRRGPDAPGAAELVAELAAAGAQVQVVACDAADRAALAKVIADIPVQHPLSGVIHTAGALDDAVVMSLTPDRVDVVLRSKVDAAWHLHELTRDLDVSAFVMFSSMAGLVGSSGQANYAAANSFLDALAAHRRAHGLPAISLGWGLWDQASAMTGGLDAADLARLGREGVLALSTAEALELFDTAMIVDEPFLAPARIDLTALRAHAVAVPPMFSDLASAPTRRQVDDSVAAAKSKSALAHRLHGLPEAEQHAVLLGLVRLHIATVLGNITPEAIDPDKAFQDLGFDSLTAVEMRNRLKSATGLSLSPTLIFDYPTPNRLASYIRTELAGLPQEIKHTPAVRTTSEDPIAIVGMACRYPGGVNSPDDMWDMLIQGRDVLSEFPADRGWDLAGLYNPDPDAAGACYTRTGGFVDGVGDFDPAFFGVGPSEALAMDPQQRMLLELSWEALERAGIDPTGLRGSATGVFAGVMTQGYGMFAAEPVEGFRLTGQLSSVASGRVAYVLGLEGPAVSVDTACSSSLVALHMAVGSLRSGECDLALAGGVTVNATPTVFVEFSRHRGLAPDGRCKPYAGRADGVGWSEGGGMLVLQRLSDARRLGHPVLAVVVGSAVNQDGASNGLTAPNGPSQQRVVRAALANAGLSAAEVDVVEGHGTGTTLGDPIEAQALLATYGQDRGEPGEPLWLGSVKSNMGHTQAAAGVAGVIKMVLAMRHELLPATLHVDVPSPHVDWSAGAVELLTAPRVWPAGARTRRAGVSSFGISGTNAHVIIEAVPVVPRREAGWAGPVVPWVVSAKSESALRGQAARLAAYVRGDDGLDVADVGWSLAGRSVFEHRAVVVGGDRDRLLAGLDELAGDQLGGSVVRGTATAAGKTVFVFPGQGSQWLGMGMGLHAGYPVFAEAFNTVVGELDRHLLRPLREVMWGHDENLLNSTEFAQPALFAVEVALFRLLGSWGVRPDFVMGHSIGELSAAHVAGVLSLENAAVLVAARGRLMQALPAGGAMVAVQAAEEEVRPLLSAEVDIAAVNGPASLVISGAQNAVAAVADQLRADGRRVHQLAVSHAFHSPLMDPMIDEFAAVAAGIAIGRPTIGVISNVTGQLAGDDFGSAAYWRRHIRQAVRFADSVRFAQAAGGSRFLEVGPSGGLVASIEESLPDVAVTTMSALRKDRPEPATLTNAVAQGFVTGMDLDWRAVVGEAQFVELPTYAFQRRRFWLSGDGVAADAAGLGLAASEHALLGAVIDLPASGGVVLTGRLSPSVQGWLADHSVAGVTIFPGAGFVELAIRAGDEVGCGVVDELTLAAPLVLPASGSVAVQVVVNGPDESGVRGVSVYSRGDVGTGWVLHAEGALRAGSAEPTADLAMWPPAGAVPVEVADGYQQLAERGYGYGPAFRGLTAMWRRGDEVFAEVALPADAGVSVTGFGVHPVLLDAALHAVVLSAESAERGQGSVLVPFSWQGVSLHAAGASAVRARIAPVGPSAVSIELADGLGLPVLSVASMLARPVTDQQLRAAVSSSGPDRLFEVTWSPQPSAAVEPLPVCAWGTTEDSAAVVFESVPLAGDVVAGVYAATSSVLDVLQSWLTRDGAGVLVVMTRGAVALPGEDVTDLAGAAVWGLVRSAQTEHPGRIVLVDSDAPLDDSALAAVVTTGEPQVLWRRGEVYTARVHGSRAVGGLLVPPSDRPWRLAMSTAGTFENLRLELIPDADAPLGPGQVRVAVSAIAANFRDVMIALGLYPDPDAVMGVEACGVVIETSLNKGSFAVGDRVMGLFPEGTGTVASTDQRLLVKVPAGWSHTAAATTSVVFATAHYALVDLAAARSGQRVLIHAGTGGVGMAAVQLARHLGLEVFATASKGKWDTLRAMGFDDDHISDSRSLEFEDKFRAATGGRGFDVVLDSLAGEFVDASLRLVAPGGVFLEMGKTDIRDPGVIAQQYPGVRYRAFDLFEAGPDRIAQILAELATLFGDGVLRPLPVTTFDVRCAPAALRYLSQARHTGKVVMLMPGSWAAGTVLITGGTGMAGSAVARHVVARHGVRNLVLVSRRGPDAPGAAELVAELAAAGAQVQVVACDAADRAALAKVIADIPVQHPLSGVIHTAGALDDAVVMSLTPDRVDVVLRSKVDAAWHLHELTRDLDVSAFVMFSSMAGLVGSSGQANYAAANSFLDALAAHRRAHGLPAISLGWGLWDQASAMTGGLATVDFKRFARDGIVAMSSADALQLFDTAMIVDEPFMLPAHIDFAALKVKFDGGTLPPMFVDLINAPTRRQVDDSLAAAKSKSALLQRLEGLPEDEQHAVLLDLVRSHIATVLGSASPEAIDPDRAFQELGFDSLTAVEMRNRLKSATGLALSPTLIFDYPNSAALAGYMRRELLGSSPQDTSAVAAGEAELQRIVASIPVKRLRQAGVLDLLLALANETETSGQDPALAPTAEQEIADMDLDDLVNAAFRNDDE

SEQ ID NO: 5 - protein, 364 aa, Mycobacterium tuberculosis
MTDLAKGPGKDPGSRGITYASAGVDIEAGDRAIDLFKPLASKATRPEVRGGLGGFAGLFTLRGDYREPVLAASSDGVGTKLAIAQAMDKHDTVGLDLVAMVVDDLVVCGAEPLFLLDYIAVGRIVPERLSAIVAGIADGCMRAGCALLGGETAEHPGLIEPDHYDISATGVGVVEADNVLGPDRVKPGDVIIAMGSSGLHSNGYSLVRKVLLEIDRMNLAGHVEEFGRTLGEELLEPTRIYAKDCLALAAETRVRTFCHVTGGGLAGNLQRVIPHGLIAEVDRGTWTPAPVFTMIAQRGRVRRTEMEKTFNMGVGMIAVVAPEDTTRALAVLTARHLDCWVLGTVCKGGKQGPRAKLVGQHPRF

SEQ ID NO: 6 - protein, 107 aa, Mycobacterium tuberculosis
MIFIVVKFETKPEWTERWPDLVASFTAATRAEEGNLWFEWSRSLDDPAEYVLVESFRDGEAGGVHVNSDHFRQAMRELPKALASTPKIISQTIDATGWSAMGEMTVG

SEQ ID NO: 7 - protein, 653 aa, Mycobacterium tuberculosis
MRTPCSQHRRDRPSAIGSQLPDADTLDTRQPPLQEIPISSFADKTFTAPAQIRNFCIIAHIDHGKSTLADRMLQLTGVVDERSMRAQYLDRMDIERERGITIKAQNVRLPWRVDKTDYVLHLIDTPGHVDFTYEVSRALEACEGAVLLVDAAQGIEAQTLANLYLALDRDLHIIPVLNKIDLPAADPDRYAAEMAHIIGCEPAEVLRVSGKTGEGVSDLLDEVVRQVPPPQGDAEAPTRAMIFDSVYDIYRGVVTYVRVVDGKISPRERIMMMSTGATHELLEVGIVSPEPKPCEGLGVGEVGYLITGVKDVRQSKVGDTVTSLSRARGAAAEALTGYREPKPMVYSGLYPVDGSDYPNLRDALDKLQLNDAALTYEPETSVALGFGFRCGFLGLLHMEITRERLEREFGLDLISTSPNVVYRVHKDDGTEIRVTNPSDWPEGKIRTVYEPVVKTTIIAPSEFIGTIMELCQSRRGELGGMDYLSPERVELRYTMPLGEIIFDFFDALKSRTRGYASLDYEEAGEQEAALVKVDILLQGEAVDAFSAIVHKDTAYAYGNKMTTKLKELIPRQQFEVPVQAAIGSKIIARENIRAIRKDVLSKCYGGDITRKRKLLEKQKEGKKRMKTIGRVEVPQEAFVAALSTDAAGDKGKK

SEQ ID NO: 8 - protein, 637 aa, Mycobacterium tuberculosis
MGATGGTQLSFADLAHAQGAAWTPADEMSLRETTFVVVDLETTGGRTTGNDATPPDAITEIGAVKVCGGAVLGEFATLVNPQHSIPPQIVRLTGITTAMVGNAPTIDAVLPMFFEFAGDSVLVAHNAGFDIGFLRAAARRCDITWPQPQVLCTMRLARRVLSRDEAPSVRLAALARLFAVASNPTHRALDDARATVDVLHALIERVGNQGVHTYAELRSYLPNVTQAQRCKRVLAETLPHRPGVYLFRGPSGEVLYVGTAADLRRRVSQYFNGTDRRKRMTEMVMLASSIDHVECAHPLEAGVRELRMLSTHAPPYNRRSKFPYRWWWVALTDEAFPRLSVIRAPRHDRVVGPFRSRSKAAETAALLARCTGLRTCTTRLTRSARHGPACPELEVSACPAARDVTAAQYAEAVLRAAALIGGLDNAALAAAVQQVTELAERRRYESAARLRDHLATAIEALWHGQRLRALAALPELIAAKPDGPREGGYQLAVIRHGQLAAAGRAPRGVPPMPVVDAIRRGAQAILPTPAPLGGALVEEIALIARWLAEPGVRIVGVSNDAAGLASPVRSAGPWAAWAATARSAQLAGEQLSRGWQSDLPTEPHPSREQLFGRTGVDCRTGPPQPLLPGRQPFSTAG

SEQ ID NO: 9 - protein, 127 aa, Mycobacterium tuberculosis
MTPDPAMLVHLCGVQEWSHARERGGIYPESDKTGYIHLSTLEQVHLPANRLYRGRADLVLLYIDPAALDSPVRWEPGVPTDPRSMLFPHLYGPLPVRAVIGAAAYPPAGDGSFGPAPEFRSATADPT

SEQ ID NO: 10 - protein, 142 aa, Mycobacterium tuberculosis
MFLIDVNVLLAAHRGDHPNHRTVRPWFDRLLAADDPFTVPNLVWASFLRLTTNRRIFEIPSPRADAFAFVEAVNAQPHHLPTSPGPRHLVLLRKLCDEADASGDLIPDAVLGAIAVEHHCAVVSLDRDFARFASVRHIRPPI

SEQ ID NO: 11 - protein, 306 aa, Mycobacterium tuberculosis
MAISLVAHQPIPHVERPMADPPRLQLARRRRSAAGPGGNEDSLMGVALLAGPANVIMELAMPGVGYGVLESRVESGRLDRHPIKRARTTFTYVAVAVAGSDDQKAAFRRAVNKVHAQVYSTPESPVSYHAFDPELQLWVAACLYKGGVDVYRTFVGEMDDEEADHHYRAGMAMGTTLQVPPQMWPPDRAAFDRYWRQSLDRVHIDDVVRDYLYPIVALRIRGIALPGPLRRLSEGIALLITTGFLPQRFRDEMRLPWDATKQRRFDALMAVLRTVNRLMPRFVREFPFNLMLWDLDRRMRRGRPLV

SEQ ID NO: 12 - protein, 363 aa, Mycobacterium tuberculosis
MWPMKIQQAKPPVTQDTSATCPLTSTVQDSSPVAGQLGRPIGFRGLAGGCPVSPLGYESPPLPLGPDSLTWRYFGDWRGMLQGPWAGSMQNMHPQLGAAVEDHSTFFRERWPRLLRSLYPIGGVVFDGDRAPVTGVQVRDYHITIKGVDGAGRRYHALNPDVFYWAHATFFVGTLHVAERFCGGLTEAQRRQLFDEHVQWYRMYGMSMRPVPATWEEFQDYWDHMCRNVLENNFAARAVLDLTELPKPPFAQRVPDWLWAAPRKLLARFFVWLTVGLYDPPVRELMGYRWLRRDEWLHRRFGDIVRLVFALVPFRFRKHPRARAGWDRATGRIPADAPLVQTPARNLPPPDERDNPTHYCPKV

SEQ ID NO: 13 - protein, 132 aa, Mycobacterium tuberculosis
MRMYADSGPDPLPDDQVCLVVEVFRMLADATRVQVLWSLADREMSVNELAEQVGKPAPSVSQHLAKLRMARLVRTRRDGTTIFYRLENEHVRQLVIDAVFNAEHAGPGIPRHHRAAGGLQSVAKASATKDVG

SEQ ID NO: 14 - protein, 352 aa, Mycobacterium tuberculosis
MRQPFIIFRQEVMELGSLIRATNLWGYTDLMRELGADPLPFLRRFDIPPGIEHQEDAFMSLAGFVRMLEASAAELDCPDFGLRLARWQGLGILGPVAVIARNAATLFGGLEAIGRYLYVHSPALTLTVSSTTARSNVRFGYEVTEPGIPYPLQGYELSMANAARMIRLLGGPQARARVFSFRHAQLGTDAAYREALGCTVRFGRTWCGFEVDHRLAGRPIDHADPETKRIATKYLESQYLPSDATLSERVVGLARRLLPTGQCSAEAIADQLDMHPRTLQRRLAAEGLRCHDLIERERRAQAARYLAQPGLYLSQIAVLLGYSEQSALNRSCRRWFGMTPRQYRAYGGVSGR

SEQ ID NO: 15 - protein, 724 aa, Mycobacterium tuberculosis
MTELAPSLVELARRFGIATEYTDWTGRQVLVSEATLVAALAALGVPAQTEQQRNDALAAQLRSYWARPLPATIVMRAGEQTQFRVHVTDGAPADVWLQLEDGTTRAEVVQVDNFTPPFDLDGRWIGEASFVLPADLPLGYHRVNLRSGDSQASAAVVVTPDWLGLPDKLAGRRAWGLAVQLYSVRSRQSWGIGDLTDLANLALWSASAHGAGYVLVNPLHAATLPGPAGRSKPIEPSPYLPTSRRFVNPLYLRVEAIPELVDLPKRGRVQRLRTNVQQHADQLDTIDRDSAWAAKRAALKLVHRVPRSAGRELAYAAFRTREGRALDDFATWCALAETYGDDWHRWPKSLRHPDASGVADFVDKHADAVDFHRWLQWQLDEQLASAQSQALRAGMSLGIMADLAVGVHPNGADAWALQDVLAQGVTAGAPPDEFNQLGQDWSQPPWRPDRLAEQEYRPFRALIQAALRHAGAVRIDHIIGLFRLWWIPDGAPPTQGTYVRYDHDAMIGIVALEAHRAGAVVVGEDLGTVEPWVRDYLLLRGLLGTSILWFEQDRDCGPAGTPLPAERWREYCLSSVTTHDLPPTAGYLAGDQVRLRESLGLLTNPVEAELESARADRAAWMAELRRVGLLADGAEPDSEEAVLALYRYLGRTPSRLLAVALTDAVGDRRTQNQPGTTDEYPNWRVPLTGPDGQPMLLEDIFTDRRAATLAEAVRAATTSPMSCW

SEQ ID NO: 16 - protein, 791 aa, Mycobacterium tuberculosis
MVTLTIDGVEISVPKGTLVIRAAELMGIQIPRFCDHPLLEPVGACRQCLVEVEGQRKPLASCTTVATDDMVVRTQLTSEIADKAQHGVMELLLINHPLDCPMCDKGGECPLQNQAMSNGRTDSRFTEAKRTFAKPINISAQVLLDRERCILCARCTRFSDQIAGDPFIDMQERGALQQVGIYADEPFESYFSGNTVQICPVGALTGTAYRFRARPFDLVSSPSVCEHCASGCAQRTDHRRGKVLRRLAGDDPEVNEEWNCDKGRWAFTYATQPDVITTPLIRDGGDPKGALVPTSWSHAMAVAAQGLAAARGRTGVLVGGRVTWEDAYAYAKFARITLGTNDIDFRARPHSAEEADFLAARIAGRHMAVSYADLESAPVVLLVGFEPEDESPIVFLRLRKAARRHRVPVYTIAPFATGGLHKMSGRLIKTVPGGEPAALDDLATGAVGDLLATPGAVIMVGERLATVPGGLSAAARLADTTGARLAWVPRRAGERGALEAGALPTLLPGGRPLADEVARAQVCAAWHIAELPAAAGRDADGILAAAADETLAALLVGGIEPADFADPDAVLAALDATGFVVSLELRHSAVTERADVVFPVAPTTQKAGAFVNWEGRYRTFEPALRGSTLQAGQSDHRVLDALADDMGVHLGVPTVEAAREELAALGIWDGKHAAGPHIAATGPTQPEAGEAILTGWRMLLDEGRLQDGEPYLAGTARTPVVRLSPDTAAEIGAADGEAVTVSTSRGSITLPCSVTDMPDRVVWLPLNSAGSTVHRQLRVTIGSIVKIGAGS

SEQ ID NO: 17 - protein, 463 aa, Mycobacterium tuberculosis
MSPQQEPTAQPPRRHRVVIIGSGFGGLNAAKKLKRADVDIKLIARTTHHLFQPLLYQVATGIISEGEIAPPTRVVLRKQRNVQVLLGNVTHIDLAGQCVVSELLGHTYQTPYDSLIVAAGAGQSYFGNDHFAEFAPGMKSIDDALELRGRILSAFEQAERSSDPERRAKLLTFTVVGAGPTGVEMAGQIAELAEHTLKGAFRHIDSTKARVILLDAAPAVLPPMGAKLGQRAAARLQKLGVEIQLGAMVTDVDRNGITVKDSDGTVRRIESACKVWSAGVSASRLGRDLAEQSRVELDRAGRVQVLPDLSIPGYPNVFVVGDMAAVEGVPGVAQGAIQGAKYVASTIKAELAGANPAEREPFQYFDKGSMATVSRFSAVAKIGPVEFSGFIAWLIWLVLHLAYLIGFKTKITTLLSWTVTFLSTRRGQLTITDQQAFARTRLEQLAELAAEAQGSAASAKVAS

SEQ ID NO: 18 - protein, 226 aa, Mycobacterium tuberculosis
MSWPGFVVNWPPMESSVLVVASPNRLPRIDCRGGVQARRTAPDTVHLVSAAATPLGGDTMRIRVIVERGAQLRLRSAAATVALPGVDTLTSHAHWEIDVTGTLDVDLEPTVVAASARHLSHATLRLHDDGRVRLRERVQIGRCNEREGFWSSSLQADRHGRPLLRHRVELGAGSLADDVIAAPRATISELRYPATAFTDAIDARSTVLALAGGGTLSTWQADRLPG

SEQ ID NO: 19 - protein, 307 aa, Mycobacterium tuberculosis
MTAHRSVLLVVHTGRDEATETARRVEKVLGDNKIALRVLSAEAVDRGSLHLAPDDMRAMGVEIEVVDADQHAADGCELVLVLGGDGTFLRAAELARNASIPVLGVNLGRIGFLAEAEAEAIDAVLEHVVAQDYRVEDRLTLDVVVRQGGRIVNRGWALNEVSLEKGPRLGVLGVVVEIDGRPVSAFGCDGVLVSTPTGSTAYAFSAGGPVLWPDLEAILVVPNNAHALFGRPMVTSPEATIAIEIEADGHDALVFCDGRREMLIPAGSRLEVTRCVTSVKWARLDSAPFTDRLVRKFRLPVTGWRGK

SEQ ID NO: 20 - protein, 647 aa, Mycobacterium tuberculosis
MNAHVEQLEFQAEARQLLDLMVHSVYSNKDAFLRELISNASDALDKLRIEALRNKDLEVDTSDLHIEIDADKAARTLTVRDNGIGMAREEVVDLIGTLAKSGTAELRAQLREAKNAAASEELIGQFGIGFYSSFMVADKVQLLTRKAGESAATRWESSGEGTYTIESVEDAPQGTSVTLHLKPEDAEDDLHDYTSEWKIRNLVKKYSDFIAWPIRMDVERRTPASQEEGGEGGEETVTIETETLNSMKALWARPKEEVSEQEYKEFYKHVAHAWDDPLEIIAMKAEGTFEYQALLFIPSHAPFDLFDRDAHVGIQLYVKRVFIMGDCDQLMPEYLRFVKGVVDAQDMSLNVSREILQQDRQIKAIRRRLTKKVLSTIKDVQSSRPEDYRTFWTQFGRVLKEGLLSDIDNRETLLGISSFVSTYSEEEPTTLAEYVERMKDGQQQIFYATGETRQQLLKSPHLEAFKAKGYEVLLLTDPVDEVWVGMVPEFDGKPLQSVAKGEVDLSSEEDTSEAEREERQKEFADLLTWLQETLSDHVKEVRLSTRLTESPACLITDAFGMTPALARIYRASGQEVPVGKRILELNPSHPLVTGLRQAHQDRADDAEKSLAETAELLYGTALLAEGGALEDPARFAELLAERLARTL

SEQ ID NO: 21 - protein, 958 aa, Mycobacterium tuberculosis
MRSQRLAGHLSAAARTIHALSLPIILFWVALTIVVNVVAPQLQSVARTHSVALGPHDAPSLIAMKRIGKDFQQFDSDTTAMVLLEGQEKLGDEAHRFYDVLVTKLSQDTTHVQHIENFWGDPLTAAGSQSADGKAAYVQLNLTGDQGGSQANESVAAVQRIVDSVPPPPGIKAYVTGPGPLGADRVVYGDRSLHTITGISIAVIAIMLFIAYRSLSAALIMLLTVGLELLAVRGIISTFAVNDLMGLSTFTVNVLVALTIAASTDYIIFLVGRYQEARATGQNREAAYYTMFGGTAHVVLASGLTVAGAMYCLGFTRLPYFNTLASPCAIGLVTVMLASLTLAPAIIAVASRFGLFDPKRATTKRRWRRIGTVVVRWPGPVLAATLLIALIGLLALPKYQTNYNERYYIPSAAPSNIGYLASDRHFPQARMEPEVLMVEADHDLRNPTDMLILDRIAKTVFHTPGIARVQSITRPLGAPIDHSSIPFQLGMQSTMTIENLQNLKDRVADLSTLTDQLQRMIDITQRTQELTRQLTDATHDMNAHTRQMRDNANELRDRIADFDDFWRPLRSFTYWERHCFDIPICWSMRSLLNSMDNVDKLTEDLANLTDDTERMDTTQRQLLAQLDPTIATMQTVKDLAQTLTSAFSGLVTQMEDMTRNATVMGRTFDAANNDDSFYLPPEAFQNPDFQRGLKLFLSPDGTCARFVITHRGDPASAEGISHIDPIMQAADEAVKGTPLQAASIYLAGTSSTYKDIHEGTLYDVMIAVVASLCLIFIIMLGITRSVVASAVIVGTVALSLGSAFGLSVLIWQHILHMPLHWLVLPMAIIVMLAVGSDYNLLLIARFQEEIGAGLKTGMIRAMAGTGRVVTIAGLVFAFTMGSMVASDLRVVGQIGTTIMIGLLFDTLVVRSYMTPALATLLGRWFWWPRRVDRLARQPQVLGPRRTTALSAERAALLQ

SEQ ID NO: 22 - protein, 561 aa, Mycobacterium tuberculosis
  misc_feature (357)..(357): Xaa can be any naturally occurring amino acid
MFAGGGAGGLGRCVMSFVSVAPEIVVAAATDLAGIGSAISAANAAAAAPTTAVLAAGADEVSAAIAALFSGHAQAYQALSAQAAAFHQQFVQTLAGGAGAYAAAEAQVEQQLLAAINAPTQALLGRPLIGNGADGAPGTGQAGGAGGILYGNGGNGGSGAAGQAGGAGGPAGLIGHGGSGGAGGSGAAGGAGGHGGWLWGNGGVGGSGGAGVGAGVAGGHGGAGGAAGLWGAGGGGGNGGNGADANIVSGGDGGLGGAGGGGGWLYGDGGAGGHGGQGAGGGAGGAGGDGGQGGAGRGLWGTGGAGGHGGQGGGTGGPPLPGQAGMGAAGGAGGLIGNGGAGGDGGVGASGGVAGVXGAGGNAMLIGHGGAGGAGGDSSFANGAAGGAGGAGGHLFGNGGSGGHGGAVTAGNTGIGGAGGVGGDARLIGHGGAGGAGGDRAGALVGRDGGPGGNGGAGGQLYGNGGDGGPGGQGGQAFGANNIGGTGGAGGNGGPAILSGNGGNGGAGGAGGAGGAGGGAGGVGGAGGAPGTGGTLQAAVSGLVTALFGAPGQPGDTGQPG

SEQ ID NO: 23 - protein, 403 aa, Mycobacterium tuberculosis
MSEEAFIYEAIRTPRGKQKNGSLHEVKPLSLVVGLIDELRKRHPDLDENLISDVILGCVSPVGDQGGDIARAAVLASGMPVTSGGVQLNRFCASGLEAVNTAAQKVRSGWDDLVLAGGVESMSRVPMGSDGGAMGLDPATNYDVMFVPQGIGADLIATIEGFSREDVDAYALRSQQKAAEAWSGGYFAKSVVPVRDQNGLLILDHDEHMRPDTTKEGLAKLKPAFEGLAALGGFDDVALQKYHWVEKINHVHTGGNSSGIVDGAALVMIGSAAAGKLQGLTPRARIVATATSGADPVIMLTGPTPATRKVLDRAGLTVDDIDLFELNEAFASVVLKFQKDLNIPDEKLNVNGGAIAMGHPLGATGAMILGTMVDELERRNARRALITLCIGGGMGVATIIERV

SEQ ID NO: 24 - protein, 545 aa, Mycobacterium tuberculosis
MTGIDMLGNAMVEACPAEGDAPVPITPAGRPRSGQRSYPDRLDVGLLRTAGVCVLASVMAHVDVTVVSVAQRTFVADFGSTQAVVAWTMTGYMLALATVIPTAGWAADRFGTRRLFMGSVLAFTLGSLLCAVAPNILLLIIFRVVQGFGGGMLTPVSFAILAREAGPKRLGRVMAVVGIPMLLGPVGGPILGGWLIGAYGWRWIFLVNLPVGLSALVLAAIVFPRDRPAASENFDYMGLLLLSPGLATFLFGVSSSPARGTMADRHVLIPAITGLALIAAFVAHSWYRTEHPLIDMRLFQNRAVAQANMTMTVLSLGLFGSFLLLPSYLQQVLHQSPMQSGVHIIPQGLGAMLAMPIAGAMMDRRGPAKIVLVGIMLIAAGLGTFAFGVARQADYLPILPTGLAIMGMGMVCSMMPLSGAAVQTLAPHQIARGSTLISVNQQVGGSIGTALMSVLLTYQFNHSEIIATAKKVALTPESGAGRGAAVDPSSLPRQTNFAAQLLHDLSHAYAVVFVIATALVVSTLIPAAFLPKQQASHRRAPLLSA

SEQ ID NO: 25 - protein, 920 aa, Mycobacterium tuberculosis
MPSPAGRLHRIRYIRLKKSSPDCRATITSGSADGQRRSPRLTNLLVVAAWVAAAVIANLLLTFTQAEPHDTSPALLPQDAKTAAATSRIAQAFPGTGSNAIAYLVVEGGSTLEPQDQPYYDAAVGALRADTRHVGSVLDWWSDPVTAPLGTSPDGRSATAMVWLRGEAGTTQAAESLDAVRSVLRQLPPSEGLRASIVVPAITNDMPMQITAWQSATIVTVAAVIAVLLLLRARLSVRAAAIVLLTADLSLAVAWPLAAVVRGHDWGTDSVFSWTLAAVLTIGTITAATMLAARLGSDAGHSAAPTYRDSLPAFALPGACVAIFTGPLLLARTPALHGVGTAGLGVFVALAASLTVLPALIALAGASRQLPAPTTGAGWTGRLSLPVSSASALGTAAVLAICMLPIIGMRWGVAENPTRQGGAQVLPGNALPDVVVIKSARDLRDPAALIAINQVSHRLVEVPGVRKVESAAWPAGVPWTDASLSSAAGRLADQLGQQAGSFVPAVTAIKSMKSIIEQMSGAVDQLDSTVNVTLAGARQAQQYLDPMLAAARNLKNKTTELSEYLETIHTWIVGFTNCPDDVLCTAMRKVIEPYDIVVTGMNELSTGADRISAISTQTMSALSSAPRMVAQMRSALAQVRSFVPKLETTIQDAMPQIAQASAMLKNLSADFADTGEGGFHLSRKDLADPSYRHVRESMFSSDGTATRLFLYSDGQLDLAAAARAQQLEIAAGKAMKYGSLVDSQVTVGGAAQIAAAVRDALIHDAVLLAVILLTVVALASMWRGAVHGAAVGVGVLASYLAALGVSIALWQHLLDRELNALVPLVSFAVLASCGVPYLVAGIKAGRIADEATGARSKGAVSGRGAVAPLAALGGVFGAGLVLVSGGSFSVLSQIGTVVVLGLGVLITVQRAWLPTTPGRR

SEQ ID NO: 26 - protein, 265 aa, Mycobacterium tuberculosis
MTTHAVIITYLRDQTQPAVDAIGGFYRTCVLTGKALVRRPFHWREAIEQGWFITSVSLLPTLAVSIPLTVLIIFTLNILLAEFGAADISGAGAALGAVTQLGPLTTVLVIAGAGATAICADLGARTIREEIDAMEVLGIDPIHRLVVPRVVAATIVAALLNGAVITIGLVGGFVFSVFIQHVSAGAYVGTLTLVTGLPEVIISVVKSATFGLIAGLVGCYRGLTTKGGPKGVGTAVNETLVLCVIALFATNVVLTTIGVRFGTGH

SEQ ID NO: 27 - protein, 968 aa, Mycobacterium tuberculosis
MSGAPTRCSPAAVNMVATAGAPLWALSASASAHCSTKMKVSPVSWSAYNWQPGSRCTSSTARVRMSRTVSTDSGLAVSVATTTTGMLGPPGTPRVGRRDAARSLVTLLRRPWQRGEQIAVTSVADGVDGVIATRLAVMSSKTVARSGTRTSRSKATSRGASRSARSAVPRKRSRPVKGVGRPSRRHHRSLLVSTGLACGRAMRAVWMMAAKGTGGAARSIGRARDIEPGHRRDGIALVLLGLAVVVAASSWFDAARPLGAWVDALLRTFIGSAVVMLPLVAAAVAVVLMRTSPNPDSRPRLILGASLIGLSFLGLCHLWAGSPEAPESRLRAAGFIGFAIGGPLSDGLTAWIAAPLLFIGALFGLLLLAGITIREVPDAMRAMFGTRLLPREYADDFEDFADFDGDDADTVEVARQDFSDGYYDEVPLCSDDGPPAWPSAEVPQDDTATIPEASAGRGSGRRGRRKDTQVLDRIVEGPYTLPSLDLLISGDPPKKRSAANTHMAGAIGEVLTQFKVDAAVTGCTRGPTVTRYEVELGPGVKVEKITALQRNIAYAVATESVRMLAPIPGKSAVGIEVPNTDREMVRLADVLTARETRRDHHPLVIGLGKDIEGDFISANLAKMPHLLVAGSTGSGKSSFVNSMLVSLLTRATPEEVRMILIDPKMVELTPYEGIPHLITPIITQPKKAAAALAWLVDEMEQRYQDMQASRVRHIDDFNDKVRSGAITAPLGSQREYRPYPYVVAIVDELADLMMTAPRDVEDAIVRITQKARAAGIHLVLATQRPSVDVVTGLIKTNVPSRLAFATSSLTDSRVILDQAGAEKLIGMGDGLFLPMGASKPLRLQGAYVSDEEIHAVVTACKEQAEPEYTEGVTTAKPTAERTDVDPDIGDDMDVFLQAVELVVSSQFGSTSMLQRKLRVGFAKAGRLMDLMETRGIVGPSEGSKAREVLVKPDELAGTLAAIRGDGGE

SEQ ID NO: 28 - protein, 226 aa, Mycobacterium tuberculosis
MILLVPILIITLMYFMFENVPHRPGTPSGFNTACLVLLGLFPLFVMFVITAITMQRERASGTLERILTTPLRRLDLLAGYGTAFSIAAAAQATLACIVAFWFLGFDTAGSPVWVFAIAIVNAVLGVGLGLLCSAFARTEFQAVQFIPLVMVPQLLLAGIIVPRALMPTWLEWISNVMPASYALEALQQVGAHPELTGIAVRDVVVVLSFAVASLCLAAVTLRRRTS

SEQ ID NO: 29 - protein, 609 aa, Mycobacterium tuberculosis
MPSPDSRPVGTRRPSSCSRICLWCCWCATMPEFRVWAPKPALVRLDVNGAVHAMTRSADGWWHTTVAAPADARYGYLLDDDPTVLPDPRSARQPDGVHARSQRWEPPGQFGAARTDTGWPGRSVEGAVIYELHIGTFTTAGTFDAAIEKLDYLVDLGIDFVELMPVNSFAGTRGWGYDGVLWYSVHEPYGGPDGLVRFIDACHARRLGVLIDAVFNHLGPSGNYLPRFGPYLSSASNPWGDGINIAGADSDEVRHYIIDCALRWMRDFHADGLRLDAVHALVDTTAVHVLEELANATRWLSGQLGRPLSLIAETDRNDPRLITRPSHGGYGITAQWNDDIHHAIHTAVSGERQGYYADFGSLATLAYTLRNGYFHAGTYSSFRRRRHGRALDTSAIPATRLLAYTCTHDQVGNRALGDRPSQYLTGGQLAIKAALTLGSPYTAMLFMGEEWGASSPFQFFCSHPEPELAHSTVAGRKEEFAEHGWAADDIPDPQDPQTFQRCKLNWAEAGSGEHARLHRFYRDLIALRHNEADLADPWLDHLMVDYDEQQRWVVMRRGQLMIACNLGAEPTCVPVSGELVLAWESPIIGDNSTELAAYSLAILRAAEPA

SEQ ID NO: 30 - protein, 385 aa, Mycobacterium tuberculosis
MDFGALPPEVNSGRMYCGPGSAPMVAAASAWNGLAAELSVAAVGYERVITTLQTEEWLGPASTLMVEAVAPYVAWMRATAIQAEQAASQARAAAAAYETAFAAIVPPPLIAANRARLTSLVTHNVFGQNTASIAATEAQYAEMWAQDAMAMYGYAGSSATATKVTPFAPPPNTTSPSAAATQLSAVAKAAGTSAGAAQSAIAELIAHLPNTLLGLTSPLSSALTAAATPGWLEWFINWYLPISQLFYNTVGLPYFAIGIGNSLITSWRALGWIGPEAAEAAAAAPAAVGAAVGGTGPVSAGLGNAATIGKLSLPPNWAGASPSLAPTVGSASAPLVSDIVEQPEAGAAGNLLGGMPLAGSGTGMGGAGPRYGFRVTVMSRPPFAG

SEQ ID NO: 31 - protein, 687 aa, Mycobacterium tuberculosis
MAGPTAPTTAPTAIRAGGPLLSPVRRNIIFTALVFGVLVAATGQTIVVPALPTIVAELGSTVDQSWAVTSYLLGGTVVVVVAGKLGDLLGRNRVLLGSVVVFVVGSVLCGLSQTMTMLAISRALQGVGAGAISVTAYALAAEVVPLRDRGRYQGVLGAVFGVNTVTGPLLGGWLTDYLSWRWAFWINVPVSIAVLTVAATAVPALARPPKPVIDYLGILVIAVATTALIMATSWGGTTYAWGSATIVGLLIGAAVALGFFVWLEGRAAAAILPPRLFGSPVFAVCCVLSFVVGFAMLGALTFVPIYLGYVDGASATASGLRTLPMVIGLLIASTGTGVLVGRTGRYKIFPVAGMALMAVAFLLMSQMDEWTPPLLQSLYLVVLGAGIGLSMQVLVLIVQNTSSFEDLGVATSGVTFFRVVGASFGTATFGALFVNFLDRRLGSALTSGAVPVPAVPSPAVLHQLPQSMAAPIVRAYAESLTQVFLCAVSVTVVGFILALLLREVPLTDIHDDADDLGDGFGVPRAESPEDVLEIAVRRMLPNGVRLRDIATQPGCGLGVAELWALLRIYQYQRLFEAVRLTDIGRHLHVPYQVFEPVFDRLVQTGYAARDGDILTLTPSGHRQVDSLAVLIRQWLLDHLAVAPGLKRQPDHQFEAALQHVTDAVLVQRDWYEDLGDLSESRQLAATT

SEQ ID NO: 32 - protein, 868 aa, Mycobacterium tuberculosis
MAAQLPTTRTAVRTRLTRLVVSIVAGLLLYASFPPRNCWWAAVVALALLAWVLTHRATTPVGGLGYGLLFGLVFYVSLLPWIGELVGPGPWLALATTCALFPGIFGLFAVVVRLLPGWPIWFAVGWAAQEWLKSILPFGGFPWGSVAFGQAEGPLLPLVQLGGVALLSTGVALVGCGLTAIALEIEKWWRTGGQGDAPPAVVLPAACICLVLFAAIVVWPQVRHAGSGSGGEPTVTVAVVQGNVPRLGLDFNAQRRAVLDNHVEETLRLAADVHAGLAQQPQFVIWPENSSDIDPFVNPDAGQRISAAAEAIGAPILIGTLMDVPGRPRENPEWTNTAIVWNPGTGPADRHDKAIVQPFGEYLPMPWLFRHLSGYADRAGHFVPGNGTGVVRIAGVPVGVATCWEVIFDRAPRKSILGGAQLLTVPSNNATFNKTMSEQQLAFAKVRAVEHDRYVVVAGTTGISAVIAPDGGELIRTDFFQPAYLDSQVRLKTRLTPATRWGPILQWILVGAAAAVVLVAMRQNGWFPRPRRSEPKGENDDSDAPPGRSEASGPPALSESDDELIQPEQGGRHSSGFGRHRATSRSYMTTGQPAPPAPGNRPSQRVLVIIPTFNERENLPVIHRRLTQACPAVHVLVVDDSSPDGTGQLADELAQADPGRTHVMHRTAKNGLGAAYLAGFAWGLSREYSVLVEMDADGSHAPEQLQRLLDAVDAGADLAIGSRYVAGGTVRNWPWRRLVLSKTANTYSRLALGIGIHDITAGYRAYRREALEAIDLDGVDSKGYCFQIDLTWRTVSNGFVVTEVPITFTERELGVSKMSGSNIREALVKVARWGIEGRLSRSDHARARPDIARPGAGGSRVSRADVTE

SEQ ID NO: 33 - protein, 561 aa, Mycobacterium tuberculosis
MVRAFNQAGVLDVSDVHVAQRLCALAGESDERVALAVAVAVRALRAGSVCVDLLSIARVAGHDDLPWPDPADWLAAVRASPLLADPPVLHLYDDRLLYLDRYWREEEQVCADLLALLTSRRPAGVPDLRRLFPTGFDEQRRAAEIALSQGVTVLTGGPGTGKTTTVARLLALVAEQAELAGEPRPRIALAAPTGKAAARLAEAVRREMAKLDATDRARLGDLHAVTLHRLLGAKPGARFRQDRQNRLPHNVIVVDETSMVSLTLMARLAEAVRPGARLILVGDADQLASVEAGAVLADLVDGFSVRDDALVAQLRTSHRFGKVIGTLAEAIRAGDGDAVLGLLRSGEERIEFVDDEDPAPRLRAVLVPHALRLREAALLGASDVALATLDEHRLLCAHRDGPTGVLHWNRRVQAWLAEETGQPPWTPWYAGRPLLVTANDYGLRVYNGDTGVVLAGPTGLRAVISGASGPLDVATGRLGDVETMHAMTIHKSQGSQVDEVTVLMPQEDSRLLTRELLYTAVTRAKRKVRVVGSEASVRAAIARRAVRASGLRMRLQSTGCG

SEQ ID NO: 34 - protein, 1098 aa, Mycobacterium tuberculosis
MTQCASRRKSTPNRAILGAFASARGTRWVATIAGLIGFVLSVATPLLPVVQTTAMLDWPQRGQLGSVTAPLISLTPVDFTATVPCDVVRAMPPAGGVVLGTAPKQGKDANLQALFVVVSAQRVDVTDRNVVILSVPREQVTSPQCQRIEVTSTHAGTFANFVGLKDPSGAPLRSGFPDPNLRPQIVGVFTDLTGPAPPGLAVSATIDTRFSTRPTTLKLLAIIGAIVATVVALIALWRLDQLDGRGSIAQLLLRPFRPASSPGGMRRLIPASWRTFTLTDAVVIFGFLLWHVIGANSSDDGYILGMARVADHAGYMSNYFRWFGSPEDPFGWYYNLLALMTHVSDASLWMRLPDLAAGLVCWLLLSREVLPRLGPAVEASKPAYWAAAMVLLTAWMPFNNGLRPEGIIALGSLVTYVLIERSMRYSRLTPAALAVVTAAFTLGVQPTGLIAVAALVAGGRPMLRILVRRHRLVGTLPLVSPMLAAGTVILTVVFADQTLSTVLEATRVRAKIGPSQAWYTENLRYYYLILPTVDGSLSRRFGFLITALCLFTAVFIMLRRKRIPSVARGPAWRLMGVIFGTMFFLMFTPTKWVHHFGLFAAVGAAMAALTTVLVSPSVLRWSRNRMAFLAALFFLLALCWATTNGWWYVSSYGVPFNSAMPKIDGITVSTIFFALFAIAAGYAAWLHFAPRGAGEGRLIRALTTAPVPIVAGFMAAVFVASMVAGIVRQYPTYSNGWSNVRAFVGGCGLADDVLVEPDTNAGFMKPLDGDSGSWGPLGPLGGVNPVGFTPNGVPEHTVAEAIVMKPNQPGTDYDWDAPTKLTSPGINGSTVPLPYGLDPARVPLAGTYTTGAQQQSTLVSAWYLLPKPDDGHPLVVVTAAGKIAGNSVLHGYTPGQTVVLEYAMPGPGALVPAGRMVPDDLYGEQPKAWRNLRFARAKMPADAVAVRVVAEDLSLTPEDWIAVTPPRVPDLRSLQEYVGSTQPVLLDWAVGLAFPCQQPMLHANGIAEIPKFRITPDYSAKKLDTDTWEDGTNGGLLGITDLLLRAHVMATYLSRDWARDWGSLRKFDTLVDAPPAQLELGTATRSGLWSPGKIRIGP

SEQ ID NO: 35 - protein, 776 aa, Mycobacterium tuberculosis
MFVEYTKSICPVCKVVVDAQVNIRHDKVYLRKRCREHGSFEALVYGDAQMYLESARFNKPGTFPLRFQTEVRDGCPSDCGLCPDHKQHACLGLIEVNTHCNLDCPICFADSGHQPDGYAITAAQCERMLDTLVAAEGEPEVVMFSGGEPTIHKQLLEFVDAAQARPVKTVIINTNGIRLASDRRFVDQLATRNRPGHPVHIYLQFDGLDEATHRRIRGHDLRDVKQRALDNCAAAGLTVSLVAAVERGLNEHELGAVIRHGMAQPGVQPVVFQPVTHAGRHVQFDPLTRLTNSDIIACITAQLPEWFRPGDFFPVPCCFPSCRSITYLLTDGEHVVPIPRLLNVEDYLDYVSNRVIPDLAIREALENLWSASAVPGTDTMTAQLQRATAALNCAEGCGINLPEALTHLTDRVFAIVIQDFQDPYTLNVKQLMKCCVQQITPDGRLIPFCAYNSVGYREQVREQLTGVPVPDIVPNAIPLAGLLADAPHGSKQANTGGSIARLAGPTRGAPMALPPQQIKACCADAYSRDIVALLLGDSFHPGGATLTRRLADQLGLRSTGDPRRVADIAAGPGASARLLASDYGVAVDGVDISEINVKRAQAAVAQTGLTERVRFHLGDAESVPLPDDTFDALVCECAFCTFPDKNAAAQQFARILRPGGLAGITDVTVGDGGLPAELTPLAAWVACIADARTVTDYTDILEGAGLRTRHIESHDESLLDMIDRIDARITALHVAAPEILADNGIRHDSVRDFTALARAAVQTGRIGYTLMIAEKP

SEQ ID NO: 36 - protein, 97 aa, Mycobacterium tuberculosis
MDATAPLVGGTALIGYVAVLGLGYVLGAKAGRRRYEQIASTYRALTGSPVARSMIEGGRRKIANRISPDAGFVTLAEIDNQTAVVQRGVERQPKTAR

SEQ ID NO: 37 - protein, 139 aa, Mycobacterium tuberculosis
MPVGGRQHVFEKLASILGLVAAPLMLLGLSACGRSAGKTSEPTCPTEPIDAADSSTTPDPSCVVRATEINGNGSRIQTWTGSYDAAATQSGGVCGGTCNFHATVRFTVDEGQISGSVDQVYQAAMVAIATRPTSPSLAP

SEQ ID NO: 38 - protein, 142 aa, Mycobacterium tuberculosis
MFLLDANVLLAAHRGDHPNHRTVRPWFDRLLAADDPFTVPNLVWASFLRLATNRRIFEIPSPRAEAFAFVEAVTAQPHHLPTNPGPRHLMLLRKLCDEADASGDLIPDAVLAAIAVGHHCAVVSLDRDFARFASVRHIRPPL

SEQ ID NO: 39 - protein, 89 aa, Mycobacterium tuberculosis
MGLNYLGQVRAIVGDCVIHIMPMGTGVELSKLADLALDIGRSVGCSAYENDFTLPDIPTQWRNQPLGWYTQGLAPYLPGLSDPKDAAEG

SEQ ID NO: 40 - protein, 216 aa, Mycobacterium tuberculosis
MSNPQPEKVRVVVGDDHPLFREGVVRALSLSGSVNVVGEADDGAAALELIKAHLPDVALLDYRMPGMDGAQVAAAVRSYELPTRVLLISAHDEPAIVYQALQQGAAGFLLKDSTRTEIVKAVLDCAKGRDVVAPSLVGGLAGEIRQRAAPVAPVLSAREREVLNRIACGQSIPAIAAELYVAPSTVKTHVQRLYEKLGVSDRAAAVAEAMRQRLLD

SEQ ID NO: 41 - protein, 141 aa, Mycobacterium tuberculosis
MSAGPAIEVAVAFVWLGMVVAISFLEAPLKFRAAGVTLQIGLGIGRLVFRALNTVEVGFALVILAIVVVGSTPARIAAAFSVALAALAVQLIAVRPRLTRRSNQVLAGLQAPRSRGHHIYVGLEIVKVVALLVAGILLLNG

SEQ ID NO: 42 - protein, 527 aa, Mycobacterium tuberculosis
MACGVGISGCAIGSAIVLASIVAGVIDPANPGMAGLRRWLGPLSILLVLWGLRASIQWLQARLAQRGASAVIADLSGQVLTAVTARRPSQLAAQRDAAAVLITRGLDGLRPYFTGYLPTLLLAAILTPATVAVIGLYDLKSMAIVVITLPLIPIFMVLIGLATTNPSAAALAAMTAVQARLLDLIAGIPTLRALGRASGPEQRIAELSADHRRSAMATLRIAFLSALVLELLATLGVALVAVGIGLRLVFGEMSLTAGLTVLLLAPEVYWPLRRVGVQFHAAADGRTAADKAFALLGESPSPTPGRRTVTARGGVIRLERLSVRGRDGRAPYDLTADIEPGRVTVLTGRNGAGKSTTLQAIAGLTAPSSGRITVAGVDVTNLAPAAWWRQLSWLPQRPVLVPGTVRHNLVLLGPVDDLERACAAAGFDAVLDELPRGLDTVLGRGGVGLSLGQRQRLGLARALGSPAAVLLLDEPTAHLDARTEQHVLGAIVERARAGATVLVVAHRQQVAAAGDRVVEVNSDGFRR

SEQ ID NO: 43 - protein, 1400 aa, Mycobacterium tuberculosis
MAPLSRKWLPVVGAVALALTFAQSPGQVSPDTKLDLTANPLRFLARATNLWNSDLPFGQAQNQAYGYLFPHGTFFVIGHLLGVPGWVTQRLWWAVLLTVGFWGLLRVAEALGVGGPSSRVVGAVAFALSPRVLTTLGSISSETLPMMLAPWVLLPTILALRGTSGRSVRALAAQAGLAVALMGAVNAIATLAGCLPAVIWWACHRPNRLWWRYTAWWLLAMALATLWWVMALTQLHGVSPPFLDFIESSGVTTQWSSLVEVLRGTDSWTPFVAPNATAGAPLVTGSAAILGTCLVAAAGLAGLTSPAMPARGRLVTMLLVGVVLLAVGHRGGLASPVAHPVQAFLDAAGTPLRNVHKVGPVIRLPLVLGLAQLLSRVPLPGSAPRPAWLRAFAHPERDKRVAVAVVALTALMVSTSLAWTGRVAPPGTFGALPQYWQEAADWLRTHHAATPTPGRVLVVPGAPFATQVWGTSHDEPLQVLGDGPWGVRDSIPLTPPQTIRALDSVQRLFAAGRPSAGLADTLARQGISYVLVRNDLDPETSRSARPILLHRSIAGSPGLAKLAEFGAPVGPDPLAGFVNDSGLRPRYPAIEIYRVSAPANPGAPYFAATDQLARVDGGPEVLLRLDERRRLQGQPPLGPVLMTADARAAGLPVPQVAVTDTPVARETDYGRVDHHSSAIRAPGDARHTYNRVPDYPVPGAEPVVGGWTGGRITVSSSSADATAMPDVAPASAPAAAVDGDPATAWVSNALQAAVGQWLQVDFDRPVTNAVVTLTPSATAVGAQVRRILIETVNGSTTLRFDEAGKPLTAALPYGETPWVRFTAAATDDGSAGVQFGITDLAITQYDASGFAHPVQLRHTVLVPGPPPGSAIAGWDLGSELLGRPGCAPGPDGVRCAASMALAPEEPANLSRTLTVPRPVSVTPMVWVRPRQGPKLADLIAAPSTTRASGDSDLVDILGSAYAAADGDPATAWTAPQRVVQHKTPPTLTLTLPRPTVVTGLRLAASRSMLPAHPTVVAINLGDGPQVRQLQVGELTTLWLHPRVTDTVSVSLLDWDDVIDRNALGFDQLKPPGLAEVVVLGAGGAPIAPADAARNRARALTVDCDHGPVVAVAGRFVHTSIRTTVGALLDGEPVAALPCEREPIALPAGQQELLISPGAAFVVDGAQLSTPGAGLSSATVTSAETGAWGPTHREVRVPESATSRVLVVPESINSGWVARTSTGARLTPIAVNGWQQAWVVPAGNPGTITLTFAPNSLYRASLAIGLALLPLLALLAFWRTGRRQLADRPTPPWRPGAWAAAGVLAAGAVIASIAGVMVMGTALGVRYALRRRERLRDRVTVGLAAGGLILAGAALSRHPWRSVDGYAGNWASVQLLALISVSVVAASVVATSESRGQDRMQ

SEQ ID NO: 44 - protein, 272 aa, Mycobacterium tuberculosis
MTALEVLGGWPVPAAAAAVIGPAGVLATHGDTARVFALASVTKPLVARAAQVAVEEGVVNLDTPAGPPGSTVRHLLAHTSGLAMHSDQALARPGTRRMYSNYGFTVLAESVQRESGIEFGRYLTEAVCEPLGMVTTRLDGGPAAAGFGATSTVADLAVFAGDLLRPSTVSAQMHADATTVQFPGLDGVLPGYGVQRPNDWGLGFEIRNSKSPHWTGECNSTRTFGHFGQSGGFIWVDPKADLALVVLTARDFGDWALDLWPAISDAVLAEYT

(19) United States
(12) Patent Application Publication
(10) Pub. No.: US 2010/0008922 A1
(43) Pub. Date: Jan. 14, 2010

(54) In Vivo Induced Genes of Mycobacterium Tuberculosis

(75) Inventor: Jeffrey D. Hillman, Gainesville, FL (US)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ORAGENICS, INC., Alachua, FL (US)

(21) Appl. No.: 12/293,497

(22) PCT Filed: Mar. 13, 2007
(86) PCT No.: PCT/US2007/063858

§ 371 Date: Sep. 2, 2009

Related U.S. Application Data
(60) Provisional application No. 60/781,953, filed on Mar. 13, 2006.

(51) Int. Cl. A61K 39/395; G01N 33/569; C07K 16/12; A61K 39/02; C07K 14/35

(52) U.S. Cl. 424/139.1; 435/7.32; 530/387.9; 424/190.1; 530/350

(57) ABSTRACT

The invention provides compositions and methods for the detection of Mycobacterium tuberculosis.

### PRIORITY

This application claims the benefit of U.S. Provisional Application No. 60/781,953, filed Mar. 13, 2006, which is incorporated by reference in its entirety herein.

### GOVERNMENT INTERESTS

This invention was made with government support under Contract No. 1R43AI055082-01A1 by the National Institutes of Health. The government has certain rights in the invention.

### BACKGROUND OF INVENTION

Tuberculosis (TB) is the world's leading cause of death from an infectious agent. The World Health Organization (WHO) estimates that there are 8 million new cases annually and two to three million deaths. Current methods for diagnosis and treatment, particularly of multiple drug resistant strains, are inadequate. In 1993, WHO took the unprecedented step of declaring tuberculosis a global emergency. Mycobacterium tuberculosis, the cause of tuberculosis, is a difficult microorganism to study using conventional methods. Consequently, there is still much to be learned about the ways it causes disease.

TB is one of the world's oldest infectious diseases, as skeletal remains show prehistoric humans had TB as early as 4000 BC. Its etiologic agent, Mycobacterium tuberculosis, however, was only identified in 1882. It was not until 1946 with the development of the antibiotic streptomycin that treatment rather than prevention became a possibility. Prior to 1946, only the sanatoria and surgical intervention were possible as supposed treatments. Any possibility that the disease could be completely eliminated with the use of drugs was dashed by the discovery of drug resistant strains in the 1980s. The increase in resistant strains could relate to the high numbers of patients who failed to complete their course of drugs. The resurgence of tuberculosis resulted in the declaration of a global health emergency by the World Health Organization in 1993.

Ninety percent of patients infected with TB have asymptomatic, latent TB infection (LTBI). There is a ten percent lifetime chance that LTBI will progress to active TB disease which, if left untreated, will kill more than fifty percent of its victims. TB is one of the top three infectious killing diseases in the world along with malaria and HIV/AIDS. Although TB is generally considered a problem only for developing countries, public health officials estimate that 10 to 15 million Americans are infected with the latent form of the disease. About one in ten people with latent TB becomes sick and contagious. If left untreated, he or she will infect on average up to 15 others each year.

Despite the overriding global need for better and simpler TB treatment regimens, no new class of TB drugs have been developed since the 1960s. TB has become the number one killer of HIV/AIDS patients and is rapidly mutating into drug resistant forms. The only available vaccine for TB has limited efficacy. Although TB drugs are inexpensive, the treatment regimens are long and complicated. Current TB therapy is based on four drugs discovered over forty years ago that must be administered for six to eight months, often under the direct observation of a healthcare provider. Due to the above complexities, global progress in controlling TB has slowed, drug resistance is spreading and TB deaths are increasing, especially in areas with high HIV prevalence. New diagnostic tests and vaccines could help significantly to control this disease.

M. tuberculosis is one of the longest studied bacteria, dating back to its isolation by Robert Koch in 1882. There was considerable early promise in prevention and treatment of tuberculosis with the isolation and implementation of the bacillus Calmette-Guérin (BCG) vaccine and the discovery of efficient antituberculosis drugs in the 1940s. The reemergence of tuberculosis has depended on a variety of factors (reviewed by Pelicic et al., 1998), chief among which are the emergence of AIDS, the low efficiency of the BCG vaccine against pulmonary tuberculosis, worsening social conditions, and resistance of up to 15% of clinical isolates to one or more front-line antituberculosis drugs (WHO, 1997). In 1993, the WHO declared this disease a global emergency (WHO, 1994).

Tuberculosis is principally a pulmonary disease, but organs other than the lungs may become infected. Primary exposure is usually via droplet transmission, and exposure to M. tuberculosis probably results only rarely in overt infection (Bloom and Small, 1998), an effect that is attributed in part to variability in the human genes that control host defenses (Casanova and Abel, 2002). The actual ratio of infection to exposure cannot be ascertained because occurrences of individual exposure cannot be accurately determined (Casadevall et al., 2000), but rates of 5-10% have been proposed (Pine, 2002).

From the standpoint of basic microbiological and immunological research, the current worsening situation with regard to the tuberculosis epidemic can be ascribed to several main factors that largely reflect the unusual nature of Mycobacteria. First, with regard to diagnosis of tuberculosis, M. tuberculosis is very slow growing in vitro. Diagnosis of tuberculosis is still a major problem. The sensitivity of sputum smears for acid-fast bacteria is only approximately 50%, and the slow growth of M. tuberculosis in vitro requires a relatively long turnaround time that impacts on important factors such as when to initiate treatment and the potential for spread of the pathogen to uninfected contacts. Despite all of the recent advances in molecular techniques, the presence of acid-fast bacteria in sputum smears and a positive culture remain the gold standard for diagnosis of tuberculosis (Chan et al., 2000). The diagnosis of asymptomatic tuberculosis infection has long depended largely upon delayed type hypersensitive reaction to a purified protein derivative (PPD) of M. tuberculosis. Tuberculin (or like) skin testing is virtually the only means to identify latent tuberculosis infection, since by definition, all culture material must be negative to qualify as latent infection (Chan et al., 2000). PPD tests are unreliable, giving significant numbers of false negatives, especially in young and elderly subjects and in any subject who is immunosuppressed. It also gives significant numbers of false positives in subjects who have received the BCG vaccine, and is therefore typically not used in such subjects.

Development of a non-bacteriological rapid diagnostic test that is both sensitive and specific for active tuberculosis has been a formidable problem. The difficulties are largely due to the inability of many tests to differentiate latent infection from active disease, the fact that most individuals, worldwide, have been vaccinated with BCG and because of exposure to saprophytic, nontuberculous mycobacteria (Chan et al., 2000; Daniel and Janicki, 1978). Several serological assays have been developed in the recent past and been found to have a high negative predictive value, making them potentially useful in ruling out active tuberculosis (reviewed by Chan et al., 2000; Gennaro, 2001). However, in populations where the prevalence of latent tuberculosis infection is high, the relatively low positive predictive value of the tests reduces their usefulness (Zahrani et al., 2000). These tests also require significantly greater costs and training, which is of particular concern in their application to third world populations that have the greatest need for such tests. Landowski and coworkers (2001) described a test based on immunodetection of circulating M. tuberculosis proteins shed during active infection. Their test used a monoclonal antibodies directed against a particular epitope of the Ag85 protein involved in transport, and also looked at several other potential shed proteins alone and in combination. They found that Ag85 performed the best, although the overall sensitivity and specificity of the test were not significantly better than certain host immune response-dependent serological tests and were slightly lower than non-culture nucleic amplification tests. They raise the possibility of adding additional epitopes from the same or other shed proteins to improve the quality of their host immune response-independent test. In summary, with regard to diagnosis, a simple, rapid, highly sensitive and specific test that would enable differentiation of healthy and latently infected subjects from actively infected subjects, regardless of whether or not they had been vaccinated with BCG, would be highly desirable, as would the identification of novel targets for the development of new, more effective vaccines.

From the standpoint of its pathophysiology, M. tuberculosis has evolved some remarkable traits to help assure its survival in the host and its transmission to new hosts. Primary infection is usually a self-limited process, reflecting the host's immune response to the pathogen (Casanova and Abel, 2002). During this period, M. tuberculosis will be taken up by host macrophages by one or more mechanisms (reviewed by Pieters, 2001) and, once intracellular, it prevents phagosome maturation and fusion with lysosomes. This is accomplished by altering the normal signal trafficking of the macrophage involving, at least in part, the phagosomal coat protein, TACO. M. tuberculosis has other effects on the local and systemic host immune response. Using the mouse model of infection, it's been shown that, in some of the macrophages constituting the granuloma, M. tuberculosis secretes one or more antigens that stimulate production of the Fas ligand (Mustafa et al., 1999). This ligand initiates apoptosis in cytotoxic T lymphocytes, thereby protecting the host macrophage from being destroyed by them. This allows the M. tuberculosis to persist in the macrophage. When local necrosis becomes significant, it appears that reduced oxygen tension serves as a signal for M. tuberculosis to enter into a nonreplicating persistence stage (reviewed by Wayne and Sohaskey, 2001). This involves down-regulation of most central intermediary metabolism processes, except for the glyoxylate shunt and several other key pathways that provide basal levels of metabolism and increased protection against host immune mechanisms. The pathogen can persist in this state, without replication, for decades. Clinically, this corresponds to the latency period. Various signals, particularly factors that depress the immune system, can lead to up-regulation of the dormant M. tuberculosis cells and reactivation of the disease process. Various animal models and cell culture models have provided much of our current information. There is still much to be learned. For example, the oxygen sensors and global regulators that lead to hypoxia-induced nonreplicating persistence have not been identified. It is possible that further identification of factors and events involved with granuloma formation and the shiftdown to and shiftup from nonreplicating persistence will identify targets for development of new drugs and diagnostic strategies.

In a market dominated by drugs developed in the 1960s with long and complicated treatment regimens, a vaccine with limited efficacy, and an old and slow testing regimen, a technology capable of identifying in vivo induced (IVI) genes in M. tuberculosis, with the ability to quickly and accurately diagnose, to provide surrogate markers for drug development, and to potentially produce an effective new vaccine, would be extremely interesting to the medical community. Increasing TB bacterial resistance to current drugs is a major concern for the medical community, and this would get their attention, not only for the ability to aid in the diagnosis of the disease stage, but also for the fact that it could potentially generate a new and more effective vaccine against M. tuberculosis.

With regard to current laboratory tests, AFB smears and cultures are the gold standard and are used to determine whether one has an active M. tuberculosis infection, an infection due to another member of the Mycobacterium family, or TB-like symptoms due to another cause. The culturing step can take weeks before a result is available and sputum smears are unreliable because they often fail to identify AFB even when demonstrated in culture. Several other testing methods, based on genetic components of mycobacteria, have been developed to help decrease the amount of time necessary to diagnose tuberculosis. These include genetic probes and molecular TB testing. They amplify/replicate pieces of the microorganism's genetic code to detect mycobacteria in body samples in less than 24 hours and can narrow the identification to a complex of mycobacteria (a combination, of which M. tuberculosis is the most common). This test is costly and requires high maintenance and calibration of equipment to meet the necessary standards for routine use. It is not likely to be useful in developing nations because of these drawbacks.

The pathogenesis of bacterial infections is a complex and dynamic process that is constantly evolving within the host. In many instances, the production of virulence determinants is tightly regulated, and their production is modulated in response to the changing environment encountered at the site of infection. It is unlikely that all regulated virulence determinants of a pathogen can be identified in vitro because it is technically impossible to determine and mimic all of the complex and changing environmental stimuli that occur at the site of an infection. This shortcoming hampers our complete understanding of the virulence mechanisms employed by human pathogens, including Mycobacterium tuberculosis. To overcome this problem, a number of investigators have emphasized the need to study bacterial virulence using organisms engaged in an actual infectious process (Smith et al., 1998; Mekalanos, 1992; Finlay and Falkow, 1997; Mahan et al., 1993; Handfield and Levesque, 1999).

Since the early 1990's, novel technologies have been designed to study gene regulation of microorganisms in vivo, hoping to fill gaps in our understanding of bacterial pathogenicity mechanisms. New approaches to identify genes specifically induced during an actual infectious process, so called “in vivo induced (IVI) genes” have shed light on various infection processes in animal models of human infections, plant-pathogen interactions, bacteria-matrix associations in biofilms, and bioremediation, as reviewed by Handfield and Levesque (1999) and Cotter and Miller (1998). All of these methods depend on the very reasonable assumption that genes expressed during in vivo growth but not during routine in vitro growth are significantly more likely to be important to the pathogenic process or, at the very least, to survival in the host. The use of technologies including in vivo expression technology (IVET), signature-tagged mutagenesis (STM), differential display, proteomic technologies, differential fluorescence induction (DFI) and diverse macro- and micro-scale differential hybridization protocols have proven to be powerful tools that have been applied to an ever-growing number of pathogenic microorganisms. Pathogenicity islands were characterized with these new tools (Hensel et al., 1997), novel targets for active and passive vaccine strategies reported (Wang et al., 1996), the global role for DNA adenine methylation (Dam) in virulence established (Heithoff et al., 1999), and new targets for antibiotic therapy were initially discovered from the study of IVI genes and proteins (Stanislavsky and Lam, 1997). In Salmonella typhimurium and Pseudomonas aeruginosa, mutant analysis has been used to confirm that certain genes identified by these methods from various pathogens do, in fact, encode virulence factors as assayed in animal models of infection (Mahan et al., 1993; Wang et al., 1996; Heithoff et al., 1999; Handfield and Levesque, 1999; Lehoux et al., 2000).

Although remarkably powerful and innovative, all of these technologies still present certain limitations, which are summarized in Table 1. One drawback for essentially all of them is that they depend on the use of animal models to obtain cells of the pathogen growing in an actual site of infection. In most instances, the animal model does not closely resemble the condition found within the natural human host. Consequently, a number of examples exist in the literature of erroneous conclusions being drawn by extrapolation of results from animal models to humans (e.g., see Smith, 1998). Furthermore, many of these schemes are not readily applicable to genetically “undomesticated” microorganisms, which are microorganisms for which there is no well established or reliable means for genetic manipulations. With the sole exception of a modified IVET approach (Slauch and Camilli, 2000), which was technically difficult to perform, none of these methods is very well suited to identify IVI genes that are transiently expressed during the course of an infection. Finally, these methods are generally restricted to the use of a single representative strain of the pathogen since, from a technical standpoint, the use of multiple strains is very demanding. This restriction can be particularly important in instances where a pathogenic species demonstrates strain-specific differences in pathogenic potential (so-called clonality).

TABLE 1
Relevant features of IVIAT

| | Other | |
|---|---|---|
| | technologies | IVIAT |
| No animal model required | No | Yes |
| Direct relevance to natural host | No | Yes |
| No genetic manipulations in the | No | Yes |
| pathogen are required | | |
| Applicable to both prokaryotic and | No | Yes |
| eukaryotic pathogens | | |
| Multiple strains and clonal types | No | Yes |
| can be analyzed simultaneously | | |
| Detects transient gene expression | No | Yes |
| Potential to detect stage and/or | No | Yes |
| route of infection-specific genes | | |
| Technically simple, fast and | No | Yes |
| inexpensive | | |

A novel approach called in vivo induced antigen technology (IVIAT; U.S. Ser. No. 09/980,845; U.S. Ser. No. 10/092,243; Handfield et al., 2000) has been described that accomplishes the same goals as IVET, STM, DFI and microarrays in identifying IVI genes. IVIAT overcomes all of the problems described above and, in particular, does not require the use of potentially misleading animal models. IVIAT (Handfield et al., Trends Microbiol. 8:336-339, 2000) identifies genes of pathogenic bacteria that are specifically expressed during actual human infections. Thus far, IVIAT has been successfully used to analyze a number of pathogenic microorganisms including Actinobacillus actinomycetemcomitans (Handfield et al., 2000; Cao et al., 2004; Handfield et al., submitted), Candida albicans (Cheng et al., 2003; Cheng et al., submitted), Porphyromonas gingivalis (Song et al., 2002), Vibrio cholerae (Hang et al., 2003), Escherichia coli (Manohar et al., in press), V. vulnificus (Kim et al., 2003), Pseudomonas aeruginosa (J. D. Hillman, unpublished), and Burkholderia pseudomallei (S. Tumwasorn, unpublished), among others. In the cases of Pseudomonas and Actinobacillus, the entire genomes have been screened with approximately 2× coverage, involving 500,000 and 200,000 independent clones, respectively.

There are several points that have emerged from these previous studies that are critical to the proper appreciation and understanding of the IVIAT method. Firstly, of the microorganisms studied so far, it is clear that humans respond to a very broad array of proteins expressed by the pathogen. This appears to be the case regardless of whether the antibodies serve a protective role or are simply so-called “bystander” antibodies (Jensen and Kapp, 1986). As many as 33% of the clones in the expression library reacted with pooled, unadsorbed serum from infected patients. In accord with this finding, Western blots in which lysates of in vitro grown cells are probed with unadsorbed serum show many hundreds of reactive bands. Thus, contrary to a priori speculations, the human humoral response is directed against much more than just surface proteins of the pathogen. This enables IVIAT to identify a broad array of IVI genes, regardless of the cellular localization of their expressed products.

Secondly, an IVI gene is not necessarily a virulence gene, nor is it a forgone conclusion that it is absolutely indispensable for survival in vivo. In the case of both diagnostic and vaccine strategies, neither of these attributes is essential, although in the latter case it might be highly preferable. Thus, the sole purpose of IVIAT is to identify IVI genes, which are more likely than in vitro and constitutively expressed genes to be important to the microorganism's pathogenic personality and which likely would not be found using conventional methods. Once the IVI genes of a pathogen have been identified, conventional biochemical, genetic, and/or immunological methods can be applied to prioritize them with regard to the ultimate goals of the project.

Thirdly, as in the case of all other in vivo expression technologies, IVIAT will not identify every virulence factor expressed by a particular pathogen. Obviously, it would not identify those virulence factors that are expressed during in vitro as well as in vivo growth. These, we presume will be discovered using conventional in vitro methods. Also, there may be proteins that, for whatever reason, are not immunogenic. Clearly, however, IVI genes that IVIAT does identify would almost certainly not be found by conventional in vitro biochemical, genetic or immunological methods. And, because IVIAT does not depend on animal models of infection, and because of its speed and ease of application to any microorganism, its ability to identify transiently expressed genes, and its flexibility with regard to potential clonality problems, IVIAT is significantly superior to other reported in vivo expression technologies.

From the standpoint of the quality of IVI genes recovered, there is very clear evidence for the superiority of IVIAT relative to microarrays and other methods. For example, V. cholerae has been extensively studied by a variety of methods intended to identify IVI genes, including microarrays. IVIAT analysis of a partial V. cholerae genomic library was performed (Hang et al., Proc. Natl. Acad. Sci., 2003). The adsorption method did not significantly remove antibodies in pooled convalescent patient sera directed against such proteins as CtxB, MshA and TcpF, which are known by other methods to be repressed during in vitro growth and expressed during human infection. Screening of the partial expression libraries for reactivity with the adsorbed, convalescent sera yielded 38 reproducibly reactive clones. In addition to identifying 27 genes previously identified in a neonatal mouse model using RIVET, STM or microarrays (Lee et al. 2001; Hensel et al., 1995; Xu et al., 2003; Merrell et al., 2002), IVIAT identified additional genes such as mshO, mshP, cheA, cheR, luxP, and four hypothetical open reading frames. Significantly, 3 of the 4 hypothetical open reading frames identified by IVIAT are encoded on chromosome II, which raises the possibility that these genes may encode functions specifically required for growth in the human intestine. Thus, IVIAT appears to identify putative pathogenesis genes that are expressed during human infection but were not previous identified using extensive animal model experiments. This finding provides an extremely clear indication of one of the main advantages of IVIAT, namely that the use of animal models is likely to lead to misleading and incomplete results, particularly since many such models circumvent the natural mode of transmission. For example, induction of adhesins necessary for colonization of a gastrointestinal pathogen are likely to be missed in a rabbit ileal loop model since conditions for their induction are unlikely to be present. Thus, to be able to identify IVI genes directly in infected humans during the course of a natural infection is an enormous advantage over other methods. Even in rare instances where a sufficient number of cells can be recovered from infected human subjects, microarray analysis is often likely to be inadequate (Schoolnik, 2002; Selinger et al., 2003). This is very well demonstrated by comparison of the results of Merrell et al. (2002), who performed transcriptional profiling of V. cholerae in human stool. IVIAT identified many of the genes that were strongly expressed in human stool, but the genes from chromosome II of V. cholerae identified by IVIAT were expressed poorly in human stool according to microarray analysis (Bina et al., 2003). One possibility is that genes on chromosome II turn off expression quickly during the transit from the upper gastrointestinal tract to stool. Since these same proteins are likely involved in generating protective immune responses, IVIAT clearly provides data superior to microarray technology by identifying relevant antigens expressed in vivo. IVIAT is being used in a more complete screen of the V. cholerae genome.

Fourthly, one of the truly remarkable aspects of IVIAT is its simplicity. Years of experience in growing and working with a particular pathogen are not required to successfully accomplish the screening aspects that lead to identification of IVI genes. Cox and coworkers (1999) and Camacho and coworkers (1999) invested a number of years (R. Jacobs, personal communication) to construct a library in M. tuberculosis in order to perform signature tagged mutagenesis. Their disease model employed intravenous injections of the library in murine models, and resulted in the recovery of a total of 13 clustered open reading frames, 7 of which are believed to be involved in the biosynthesis of the mycobacterial cell envelope. By contrast, our work took 6 months to screen the entire M. tuberculosis genome to recover and identify 44 IVI genes that operate in various aspects of cellular physiology and genetics, as described below. The TB Research Materials and Vaccine Testing Center at Colorado State University facilitated our studies by providing a ready source of cells, cell lysates, and DNA. The World Health Organization provided 80 serum samples from infected patients in different stages of disease, and included basic information regarding each subject's health status, prior BCG vaccination, and results of staining sputum samples for acid fast bacilli. These are all of the essential reagents for performing the initial screening and characterization of the IVI genes recovered, and required no special knowledge of M. tuberculosis genetics, physiology, or immunobiology.

Fifthly, we also do not assume that any gene is totally shut off under any in vitro growth condition, since there may be a basal level of expression. Our experience with IVIAT indicates that, while our adsorption process may possibly exhaust the sera of antibodies directed at certain differentially expressed genes, this is not the general case. Through trial and error, our adsorption process has been refined to achieve an excellent balance between eliminating antibodies directed against in vitro induced antigens and preserving antibodies directed against IVI antigens. The fact that we have already identified a significant number of interesting IVI genes from a broad array of human pathogens is the strongest argument that IVIAT works and yields marketable results.

Finally, antibody probes from infected animals or humans have been used in the past to study bacterial virulence (e.g., Aitchison et al., 1987; Lyashchenko et al., 1998a; Yi et al., 1997; Li et al., 2000). Because the targets of these probes were in vitro grown cells, these studies were designed to identify genes that were constitutively expressed or regulated genes that happened to be expressed under the conditions of in vitro cultivation that were used. IVIAT is clearly and expressly different from these sorts of studies. By adsorbing out antibodies directed against in vitro expressed genes and by probing a genomic expression library of the pathogen's DNA, IVIAT identifies genes of the pathogen that are specifically expressed in vivo, and in particular, during infection of an actual human host. There is currently no other technology that can achieve this end.

Genes specifically expressed during the persistence phase may be identified by IVIAT using serum from patients in that phase of infection, and these genes may serve as potential markers for use in diagnostic tests. In general, application of IVIAT to M. tuberculosis is very likely to isolate genes that are involved in various important functions as they occur in the human host during primary, recurrent and nonreplicating stages of infection.

IVI genes are also very likely to provide interesting and novel targets for vaccine approaches. With regard to vaccine strategies, meta-analysis of the BCG vaccine over decades of application has shown a lack of effectiveness (Coldiz, 1995; Behr, and Small, 1997). Like other intracellular pathogens that are predominantly controlled by T lymphocytes, there is no consistently effective vaccine that has so far been found. This has suggested the need for novel approaches including DNA vaccines (reviewed by Sharma and Khuller, 2001) and the use of live attenuated vaccines or vaccine carrier strains (reviewed by Mollenkopf et al., 2001). And, there certainly are a number of subunit type vaccines that have also been developed. All of these categories of vaccines were recently reviewed by Orme et al. (2001). At the time of their publication, 170 candidate or combinations of candidates were tested first in the mouse model and, if found to be promising, in the guinea pig model. The authors emphasize that it is a big jump from animals to humans. Indeed, these animal models predicted that BCG should have a positive effect, which we know from extensive human testing is only partially true. M. tuberculosis is not readily conducive to genetic manipulations (reviewed by Pelicic et al., 1998; Tang et al., 2001), although, as mentioned above, some progress has been made that has enabled the identification of several IVI genes using signature tagged mutagenesis. The two groups (Camacho et al, 1999; Cox et al., 1999) that independently reported attenuating insertions in a gene cluster that contained ORFs involved in cell envelope biosynthesis. A mutation in one of these genes demonstrated an altered pathogenic potential in an animal model, thereby directly demonstrating that IVI genes can serve as good, potential targets for novel antibiotics and/or for use in live attenuated strains for vaccine applications. These results accord with the widely held belief that IVI genes are among the preferred targets for vaccine approaches to a variety of microbial infections (reviewed by Handfield and Levesque, 1999), since their products are likely to be involved in the pathogenic process.

The resurgence of tuberculosis has emphasized the need for new diagnostic tests, vaccines and drugs. By more accurately and rapidly determining whether a person has been infected, and whether the disease is in an active or latent stage, treatment can be better directed and potentially save millions of lives and substantially reduce the overall cost of curing each case of the disease. A simple, rapid, highly sensitive and specific test that would enable differentiation of healthy and TB-infected subjects regardless of whether or not they had been vaccinated with BCG is needed.

The successful completion of the development of a diagnostic TB test is expected to address a critical healthcare need through its ability to differentiate healthy and latently infected subjects from actively infected subjects. As noted previously, the identified in vivo expressed proteins of M. tuberculosis may have application both as a diagnostic and as a vaccine. Thus, the identification of IVI genes offers the promise of a new diagnostic TB test to meet a critical need, particularly in developing countries, and could potentially lead to the development of a more effective TB vaccine.

### SUMMARY OF THE INVENTION

In one embodiment, the invention provides a method for detecting an antibody specific for Mycobacterium tuberculosis in a test sample comprising contacting the test sample with a purified polypeptide comprising SEQ ID NOs:1-44 or a combination thereof and detecting formation of an immunocomplex comprising the polypeptide of SEQ ID NO:1-44 and the antibody specific for M. tuberculosis, wherein detection of the immunocomplex indicates the presence of an antibody specific for M. tuberculosis in the test sample. The test sample can be blood, sputum, serum, or lung lavage fluid. The polypeptide can be immobilized on a substrate. The method can comprise an assay selected from the group consisting of a radioimmunoassay, horizontal flow chromatography, a dot blot assay, a competitive-binding assay, a western blot, an enzyme-linked immunosorbent assay (ELISA), and a sandwich assay.

In another embodiment, the invention provides a method for diagnosing a patient with tuberculosis comprising contacting a test sample from the patient with a purified polypeptide comprising SEQ ID NOs:1-44 and detecting formation of an immunocomplex comprising the polypeptide of SEQ ID NO:1-44 and the antibody specific for M. tuberculosis, wherein detection of the immunocomplex indicates that the patient has tuberculosis. The detection of the immunocomplex can indicate that the patient has tuberculosis whether or not the patient has been vaccinated for tuberculosis.

In yet another embodiment, the invention provides a method of detecting the presence or absence of a M. tuberculosis antigen in a test sample comprising contacting the test sample with an antibody or an antigen-binding portion thereof that specifically binds to a polypeptide consisting of SEQ ID NO:1-44, and detecting an immunocomplex comprising the M. tuberculosis antigen and the antibody or antigen-binding portion thereof, wherein detection of the immunocomplex indicates the presence of the M. tuberculosis antigen in the test sample.

Still another embodiment of the invention provides a purified antibody or antigen-binding portion thereof that binds to a polypeptide consisting of SEQ ID NOs:1-44 with a binding affinity of about Kₐ of 10⁷ l/mol or more. Another embodiment of the invention provides a composition comprising the purified antibody or antigen-binding portion thereof and a pharmaceutically acceptable carrier.

Even another embodiment of the invention provides a method of determining effectiveness of a treatment for a M. tuberculosis infection comprising:

(a) determining an amount or presence of one or more polypeptides comprising SEQ ID NOs:1-44 in a test sample from a patient infected with M. tuberculosis prior to treatment;

(b) determining an amount or presence of the one or more polypeptides comprising SEQ ID NOs:1-44 in a test sample from the patient after one or more treatments; and

(c) comparing the amount or presence of the one or more polypeptides comprising SEQ ID NOs:1-44 in steps (a) and (b);

wherein a lesser amount of the polypeptides in step (b) as compared to step (c) indicates effectiveness of the treatment; and wherein the absence of the polypeptides in step (b) as compared to presence of the polypeptides in step (a) indicates effectiveness of the treatment.

Another embodiment of the invention provides a method of determining effectiveness of a treatment for a M. tuberculosis infection. The method comprises:

(a) determining an amount of one or more polypeptides comprising SEQ ID NOs:1-44 in a test sample from a patient infected with M. tuberculosis;

(b) comparing the amount of the one or more polypeptides comprising SEQ ID NOs:1-44 to a standard.

If the amount of the one or more polypeptides is greater than the standard, then the treatment is ineffective and wherein if the amount of the one or more polypeptides is less than or equal to the standard then the treatment is effective.

Yet another embodiment of the invention provides an antibody that specifically binds a polypeptide consisting of SEQ ID NO:1-44.

Another embodiment of the invention provides a method of ameliorating one or more symptoms of tuberculosis comprising administering one or more antibodies of the invention to a tuberculosis patient.

Even another embodiment of the invention provides an immunogenic composition comprising one or more purified polypeptides shown in SEQ ID NO:1-44 and one or more pharmaceutically acceptable carriers. The composition can further comprise one or more adjuvants.

Still another embodiment of the invention provides a method of immunizing a mammal against a Mycobacterium tuberculosis infection comprising administering immunogenic composition of the invention to the mammal.

Yet another embodiment of the invention provides a fusion protein comprising one or more of polypeptides comprising SEQ ID NO:1-44 and a heterologous protein, wherein the heterologous protein can be a polypeptide comprising SEQ ID NOs:1-44. The heterologous protein can be Mycobacterium tuberculosis Antigen 85b, Mycobacterium tuberculosis ESAT-6, Mycobacterium tuberculosis MtB41, or Mycobacterium tuberculosis Mtb39.

Another embodiment of the invention provides an immunogenic composition comprising one or more purified polynucleotides that encode one or more polypeptides comprising SEQ ID NO:1-44 and one or more pharmaceutically acceptable carriers.

Even another embodiment of the invention provides a method of immunizing a mammal against a Mycobacterium tuberculosis infection, stimulating a Mycobacterium tuberculosis specific immunogenic response in a mammal, or reducing the severity of a Mycobacterium tuberculosis infection comprising administering the immunogenic composition of comprising one or more purified polynucleotides that encode one or more polypeptides comprising SEQ ID NO:1-44 and one or more pharmaceutically acceptable carriers to the mammal.

Still another embodiment of the invention provides a method for diagnosing a patient with a M. tuberculosis infection comprising: a) contacting a biological sample from the patient with at least two oligonucleotide primers in a polymerase chain reaction, wherein the at least two oligonucleotide primers hybridize specifically to a polynucleotide encoding a polypeptide comprising SEQ ID NOs:1-44, and wherein the oligonucleotide primers hybridize sufficiently to allow amplification of the primers; and b) detecting amplified nucleic acid sequences in the biological sample; wherein the presence of the amplified nucleic acid sequences indicates an M tuberculosis infection and the absence of the amplified nucleic acid sequences indicates absence of a M. tuberculosis infection.

Another embodiment of the invention provides a method for diagnosing a patient with a M. tuberculosis infection comprising: a) contacting a biological sample with one or more oligonucleotide probes that hybridize specifically to a polynucleotide encoding a polypeptide comprising SEQ ID NO:1-44, under high stringency conditions, under conditions sufficient to allow hybridization between any polynucleotides present in the sample and the one or more oligonucleotide probes; and b) detecting whether the one or more oligonucleotide probes hybridized to one or more polypeptides in the sample; wherein the presence of hybridization indicates M. tuberculosis infection, and the absence of hybridization indicates an absence of M tuberculosis infection.

Therefore, the invention provides polypeptides and antibodies for use in diagnostics, drug development, and vaccines. The invention can provide for a series of rapid and accurate tests, specific for primary TB, dormant infections, secondary TB, and primary or secondary TB regardless of the BCG vaccination history of the patient.

### BRIEF DESCRIPTION OF THE FIGURES

FIG. 1 shows a diagram of the IVIAT system.

FIG. 2 shows five rounds of adsorption of 500 μl of serum against whole, irradiated cells and extracts of CSU93 (CDC 1551).

### DETAILED DESCRIPTION OF THE INVENTION

In vivo induced antigen technology (IVIAT) has been used to study Mycobacterium tuberculosis pathogenesis. Like earlier technologies, such as IVET and signature tagged mutagenesis, the approach is designed to identify genes of a pathogen that are expressed only during an actual infectious process and that, therefore, are likely to be important in disease causation or in vivo survival. IVIAT uses pooled sera from patients who have tuberculosis. Antibodies in the sera are used as probes to identify genes that are expressed during in vivo but not during in vitro growth of the M. tuberculosis. There are three major conceptual and technological advantages provided by IVIAT: 1) identification of in vivo induced (IVI) genes does not rely on animal models that, in most cases, poorly mimic a natural infection in the human host. Instead, it identifies genes expressed during an actual human infection; 2) any microorganism can be readily studied, regardless of the ability to genetically manipulate it; and 3) it can identify genes that are expressed at any point in the course of infection, even if they are only transiently expressed. From the application of IVIAT to M. tuberculosis, 44 IVI genes have been identified and validated. The majority of them have been validated using Western blot analyses to demonstrate that antibodies in serum of patients react with the products expressed by the cloned IVI genes. The genes have been analyzed also with regard to their function, if known, their cellular location, and other genomic and proteomic parameters. The IVI genes can be used in the development of a test that is substantially faster and better than the current PPD test that requires an extended period for development, two visits to the caregiver, and does not differentiate between patients with tuberculosis and individuals who have received the BCG vaccine. These targets may also serve as biomarkers to determine the effectiveness of treatment once the diagnosis of active tuberculosis has been made and therapy instituted.

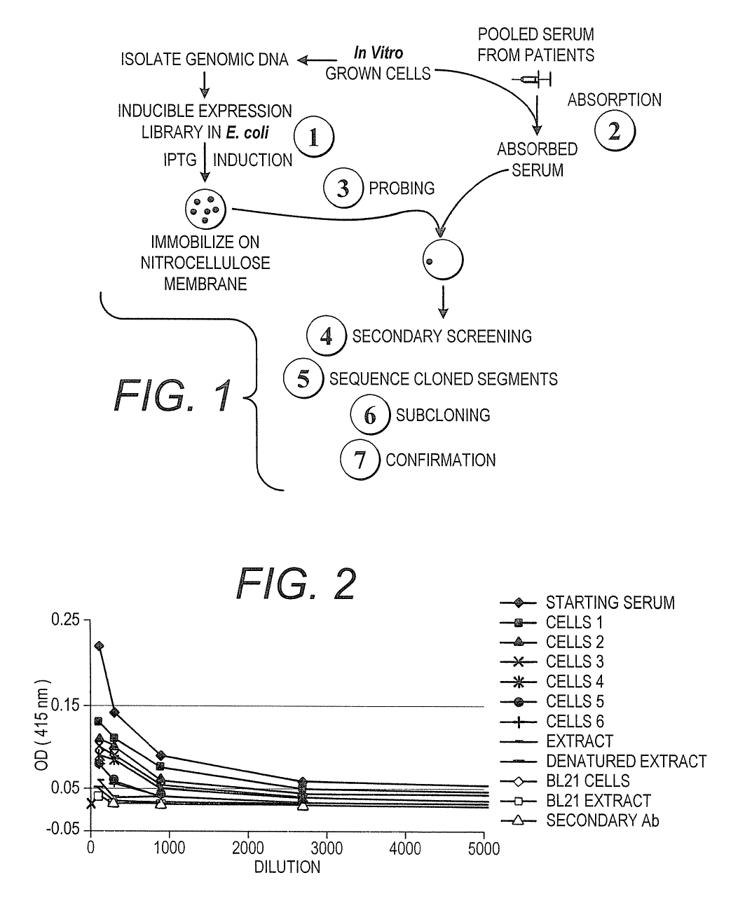

The invention provides an enormous potential breakthrough since a good animal model for studying M. tuberculosis has been a major hindrance to studying this microorganism (Balasubramanian et al., 1994; Tsuyuguchi, 2000; Smith et al., 2000). A general overview of the IVIAT scheme that was used to identify IVI genes of M. tuberculosis is presented in FIG. 1. First, an expression library of the pathogen's DNA is generated in a suitable host, such as Escherichia coli. It is possible to use DNA from one or more strains of the pathogen in library construction. Second, sera from patients who have experienced an infection caused by the pathogen under study is pooled and repeatedly adsorbed with whole cells and extracts of cells of the pathogen grown in vitro, leaving antibodies against antigens that are expressed only in vivo. Considerable attention should paid to the types and numbers of serum samples used to optimize the array of IVI genes recovered. A complete explanation of this aspect of IVIAT, particularly as it was applied to M. tuberculosis, is presented in the Examples section. Third, the adsorbed antibody probe is used in a colony lift format to identify reactive clones in the library. These are clones that are producing antigens expressed during a natural infection. In the fourth step, these clones are purified and rescreened to minimize false positives. In the fifth and sixth steps, plasmid DNA from reactive clones is sequenced and the ORF responsible for expression of an IVI antigen is subcloned. Lastly, PAGE and Western blot analysis is used in a final confirmation step.

Forty-four IVI genes were identified in the IVIAT analysis of M. tuberculosis. These genes and their expressed products have great potential for the development of new diagnostic, vaccine and biomarker strategies.

M. tuberculosis Polypeptides

A polypeptide is a polymer of three or more amino acids covalently linked by amide bonds. A polypeptide can be post-translationally modified. A purified polypeptide is a polypeptide preparation that is substantially free of cellular material, other types of polypeptides, chemical precursors, chemicals used in synthesis of the polypeptide, or combinations thereof. A polypeptide preparation that is substantially free of cellular material, culture medium, chemical precursors, chemicals used in synthesis of the polypeptide has less than about 30%, 20%, 10%, 5%, 1% or more of other polypeptides, culture medium, chemical precursors, and/or other chemicals used in synthesis. Therefore, a purified polypeptide is about 70%, 80%, 90%, 95%, 99% or more pure.

Purified polypeptides of the invention can either be full-length polypeptides or fragments of polypeptides. For example, fragments of polypeptides of the invention can comprise about 5, 10, 20, 50, 100, 200, 300, 400, 500, 600, 750, 1,000 or more amino acids of polypeptides of the invention. Examples of polypeptides of the invention include those shown in SEQ ID NOs:1-44. Variant polypeptides are at least about 90, 96, 98, or 99% identical to the polypeptide sequences shown in SEQ ID NOs:1-44. Variant polypeptides have 1, 2, 3, 4, 5, 10, 20, 30, 40, 50 or more conservative amino acid substitutions or, optionally, other minor modifications and retain biological activity, i.e., are biologically functional equivalents. A biologically active equivalent has substantially equivalent function when compared to the corresponding wild-type polypeptide.

Percent sequence identity has an art recognized meaning and there are a number of methods to measure identity between two polypeptide or polynucleotide sequences. See, e.g., Lesk, Ed., Computational Molecular Biology, Oxford University Press, New York, (1988); Smith, Ed., Biocomputing: Informatics And Genome Projects, Academic Press, New York, (1993); Griffin & Griffin, Eds., Computer Analysis Of Sequence Data, Part 1, Humana Press, New Jersey, (1994); von Heinje, Sequence Analysis In Molecular Biology, Academic Press, (1987); and Gribskov & Devereux, Eds., Sequence Analysis Primer, M Stockton Press, New York, (1991). Methods for aligning polynucleotides or polypeptides are codified in computer programs, including the GCG program package (Devereux et al., Nuc. Acids Res. 12:387 (1984)), BLASTP, BLASTN, FASTA (Atschul et al., J. Molec. Biol. 215:403 (1990)), and Bestfit program (Wisconsin Sequence Analysis Package, Version 8 for Unix, Genetics Computer Group, University Research Park, 575 Science Drive, Madison, Wis. 53711) which uses the local homology algorithm of Smith and Waterman (Adv. App. Math., 2:482-489 (1981)). For example, the computer program ALIGN which employs the FASTA algorithm can be used, with an affine gap search with a gap open penalty of −12 and a gap extension penalty of −2.

When using any of the sequence alignment programs to determine whether a particular sequence is, for instance, about 95% identical to a reference sequence, the parameters are set such that the percentage of identity is calculated over the full length of the reference polynucleotide and that gaps in identity of up to 5% of the total number of nucleotides in the reference polynucleotide are allowed.

Variants can generally be identified by modifying one of the polypeptide sequences of the invention, and evaluating the properties of the modified polypeptide to determine if it is a biological equivalent. A variant is a biological equivalent if it reacts substantially the same as a polypeptide of the invention in an assay such as an immunohistochemical assay, an enzyme-linked immunosorbent Assay (ELISA), a radioimmunoassay (RIA), immunoenzyme assay or a western blot assay, e.g., has 90-110% of the activity of the original polypeptide. In one embodiment, the assay is a competition assay wherein the biologically equivalent polypeptide is capable of reducing binding of the polypeptide of the invention to a corresponding reactive antigen or antibody by about 80, 95, 99, or 100%. An antibody that specifically binds a corresponding wild-type polypeptide also specifically binds the variant polypeptide. Variant polypeptides of the invention can comprise about 1, 2, 3, 4, 5, 10, 10, 30, 40, 50, 60, 70, 80, 90, 100 or more conservative amino acid substitutions.

A conservative substitution is one in which an amino acid is substituted for another amino acid that has similar properties, such that one skilled in the art of peptide chemistry would expect the secondary structure and hydropathic nature of the polypeptide to be substantially unchanged. Conservative substitutions include swaps within groups of amino acids such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly. A polypeptide of the invention can further comprise a signal (or leader) sequence that co-translationally or post-translationally directs transfer of the protein. The polypeptide can also comprise a linker or other sequence for ease of synthesis, purification or identification of the polypeptide (e.g., poly-His), or to enhance binding of the polypeptide to a solid support. For example, a polypeptide can be conjugated to an immunoglobulin Fc region or bovine serum albumin.

13. A polypeptide can be covalently or non-covalently linked to an amino acid sequence to which the polypeptide is not normally associated with in nature (i.e., a heterologous amino acid sequence). A heterologous amino acid sequence can be a non-M. tuberculosis polypeptide or can be a M. tuberculosis polypeptide that is not found in association with the polypeptide in nature. A heterologous polypeptide can be, e.g., Mycobacterium tuberculosis Antigen 85b, Mycobacterium tuberculosis ESAT-6, Mycobacterium tuberculosis MtB41, or Mycobacterium tuberculosis Mtb39. Additionally, a polypeptide can be covalently or non-covalently linked to compounds or molecules other than amino acids. For example, a polypeptide can be linked to an indicator reagent, an amino acid spacer, an amino acid linker, a signal sequence, a stop transfer sequence, a transmembrane domain, a protein purification ligand, or a combination thereof. In one embodiment of the invention a protein purification ligand can be one or more C amino acid residues at, for example, the amino terminus or carboxy terminus of a polypeptide of the invention. An amino acid spacer is a sequence of amino acids that are not associated with a polypeptide of the invention in nature. An amino acid spacer can comprise about 1, 5, 10, 20, 100, or 1,000 amino acids.

If desired, a polypeptide can be a fusion protein, which can also contain other amino acid sequences, such as amino acid linkers, amino acid spacers, signal sequences, TMR stop transfer sequences, transmembrane domains, as well as ligands useful in protein purification, such as glutathione-S-transferase, histidine tag, and staphylococcal protein A, or combinations thereof. More than one polypeptide of the invention can be present in a fusion protein. Fragments of polypeptides of the invention can be present in a fusion protein of the invention. A fusion protein of the invention can comprise one or more of SEQ ID NOs:1-44, fragments thereof, or combinations thereof. A fusion protein can comprise one or more of polypeptides comprising SEQ ID NO:1-44 and a heterologous protein.

Polypeptides of the invention can be in a multimeric form. That is, a polypeptide can comprise one or more copies of SEQ ID NOs:1-44 or a combination thereof. A multimeric polypeptide can be a multiple antigen peptide (MAP). See e.g., Tam, J. Immunol. Methods, 196:17-32 (1996).

Polypeptides of the invention can comprise an antigen that is recognized by an antibody specific for M. tuberculosis. The antigen can comprise one or more epitopes (i.e., antigenic determinants). An epitope can be a linear epitope, sequential epitope or a conformational epitope. Epitopes within a polypeptide of the invention can be identified by several methods. See, e.g., U.S. Pat. No. 4,554,101; Jameson & Wolf, CABIOS 4:181-186 (1988). For example, a polypeptide of the invention can be isolated and screened. A series of short peptides, which together span an entire polypeptide sequence, can be prepared by proteolytic cleavage. By starting with, for example, 100-mer polypeptide fragments, each fragment can be tested for the presence of epitopes recognized in an ELISA. For example, in an ELISA assay a M. tuberculosis polypeptide, such as a 100-mer polypeptide fragment, is attached to a solid support, such as the wells of a plastic multi-well plate. A population of antibodies are labeled, added to the solid support and allowed to bind to the unlabeled antigen, under conditions where non-specific absorption is blocked, and any unbound antibody and other proteins are washed away. Antibody binding is detected by, for example, a reaction that converts a colorless substrate into a colored reaction product. Progressively smaller and overlapping fragments can then be tested from an identified 100-mer to map the epitope of interest.

A polypeptide of the invention can be produced recombinantly. A polynucleotide encoding a polypeptide of the invention can be introduced into a recombinant expression vector, which can be expressed in a suitable expression host cell system using techniques well known in the art. A variety of bacterial, yeast, plant, mammalian, and insect expression systems are available in the art and any such expression system can be used. Optionally, a polynucleotide encoding a polypeptide can be translated in a cell-free translation system. A polypeptide can also be chemically synthesized or obtained from M. tuberculosis cells.

An immunogenic polypeptide of the invention can comprise an amino acid sequence shown in SEQ ID NOs:1-44. An immunogenic polypeptide can elicit antibodies or other immune responses (e.g., T-cell responses of the immune system) that recognize epitopes of polypeptides having SEQ ID NOs:1-44. An immunogenic polypeptide of the invention can also be a fragment of a polypeptide that has an amino acid sequence shown in SEQ ID NOs:1-44. An immunogenic polypeptide fragment of the invention can be about 5, 10, 20, 50, 100, 200, 300, 400, 500, 600, 750, 1,000 or more amino acids in length.

M. tuberculosis Polynucleotides

Polynucleotides of the invention contain less than an entire microbial genome and can be single- or double-stranded nucleic acids. A polynucleotide can be RNA, DNA, cDNA, genomic DNA, chemically synthesized RNA or DNA or combinations thereof. The polynucleotides can be purified free of other components, such as proteins, lipids and other polynucleotides. For example, the polynucleotide can be 50%, 75%, 90%, 95%, 96%, 97%, 98%, 99%, or 100% purified. The polynucleotides of the invention encode the polypeptides described above. In one embodiment of the invention the polynucleotides encode polypeptides shown in SEQ ID NOs:1-44 or combinations thereof. Polynucleotides of the invention can comprise other nucleotide sequences, such as sequences coding for linkers, signal sequences, TMR stop transfer sequences, transmembrane domains, or ligands useful in protein purification such as glutathione-S-transferase, histidine tag, and staphylococcal protein A.

Polynucleotides of the invention can be isolated. An isolated polynucleotide is a naturally-occurring polynucleotide that is not immediately contiguous with one or both of the 5′ and 3′ flanking genomic sequences that it is naturally associated with. An isolated polynucleotide can be, for example, a recombinant DNA molecule of any length, provided that the nucleic acid sequences naturally found immediately flanking the recombinant DNA molecule in a naturally-occurring genome is removed or absent. Isolated polynucleotides also include non-naturally occurring nucleic acid molecules. A nucleic acid molecule existing among hundreds to millions of other nucleic acid molecules within, for example, cDNA or genomic libraries, or gel slices containing a genomic DNA restriction digest are not to be considered an isolated polynucleotide.

Polynucleotides of the invention can also comprise fragments that encode immunogenic polypeptides. Polynucleotides of the invention can encode full-length polypeptides, polypeptide fragments, and variant or fusion polypeptides.

Degenerate nucleotide sequences encoding polypeptides of the invention, as well as homologous nucleotide sequences that are at least about 90, 96, 98, or 99% identical to the polynucleotide sequences of the invention and the complements thereof are also polynucleotides of the invention. Percent sequence identity can be calculated as described in the “M. tuberculosis Polypeptides” section. Degenerate nucleotide sequences are polynucleotides that encode a polypeptide of the invention or fragments thereof, but differ in nucleic acid sequence from the wild-type polynucleotide sequence, due to the degeneracy of the genetic code. Complementary DNA (cDNA) molecules, species homologs, and variants of M. tuberculosis polynucleotides that encode biologically functional M. tuberculosis polypeptides also are M. tuberculosis polynucleotides. Polynucleotides of the invention can be isolated from nucleic acid sequences present in, for example, a biological sample, such as blood, serum, sputum, lung lavage fluid, saliva, or tissue from an infected individual. Polynucleotides can also be synthesized in the laboratory, for example, using an automatic synthesizer. An amplification method such as PCR can be used to amplify polynucleotides from either genomic DNA or cDNA encoding the polypeptides.

Polynucleotides of the invention can comprise coding sequences for naturally occurring polypeptides or can encode altered sequences that do not occur in nature. If desired, polynucleotides can be cloned into an expression vector comprising expression control elements, including for example, origins of replication, promoters, enhancers, or other regulatory elements that drive expression of the polynucleotides of the invention in host cells. An expression vector can be, for example, a plasmid, such as pBR322, pUC, or ColE1, or an adenovirus vector, such as an adenovirus Type 2 vector or Type 5 vector. Optionally, other vectors can be used, including but not limited to Sindbis virus, simian virus 40, alphavirus vectors, poxvirus vectors, and cytomegalovirus and retroviral vectors, such as murine sarcoma virus, mouse mammary tumor virus, Moloney murine leukemia virus, and Rous sarcoma virus. Minichromosomes such as MC and MC1, bacteriophages, phagemids, yeast artificial chromosomes, bacterial artificial chromosomes, virus particles, virus-like particles, cosmids (plasmids into which phage lambda cos sites have been inserted) and replicons (genetic elements that are capable of replication under their own control in a cell) can also be used.

Methods for preparing polynucleotides operably linked to an expression control sequence and expressing them in a host cell are well-known in the art. See, e.g., U.S. Pat. No. 4,366,246. A polynucleotide of the invention is operably linked when it is positioned adjacent to or close to one or more expression control elements, which direct transcription and/or translation of the polynucleotide.

Polynucleotides of the invention can be used, for example, as probes or primers, for example PCR primers, to detect the presence of M. tuberculosis polynucleotides in a sample, such as a biological sample. The ability of such probes and primers to specifically hybridize to M. tuberculosis polynucleotide sequences will enable them to be of use in detecting the presence of complementary sequences in a given sample. Polynucleotide probes and primers of the invention can hybridize to complementary sequences in a sample such as a biological sample, including saliva, sputum, blood, serum, lung lavage fluid, urine, feces, cerebrospinal fluid, amniotic fluid, wound exudate, or tissue. Polynucleotides from the sample can be, for example, subjected to gel electrophoresis or other size separation techniques or can be immobilized without size separation. The polynucleotide probes or primers can be labeled. Suitable labels, and methods for labeling probes and primers are known in the art, and include, for example, radioactive labels incorporated by nick translation or by kinase, biotin labels, fluorescent labels, chemiluminescent labels, bioluminescent labels, metal chelator labels and enzyme labels. The polynucleotides from the sample are contacted with the probes or primers under hybridization conditions of suitable stringencies.

Depending on the application, varying conditions of hybridization can be used to achieve varying degrees of selectivity of the probe or primer towards the target sequence. Methods for hybridization are well known and design of probes and primers is well known in the art. For applications requiring high selectivity, relatively high stringent conditions can be used, such as low salt and/or high temperature conditions, such as provided by a salt concentration of from about 0.02 M to about 0.15 M salt at temperatures of from about 50° C. to about 70° C. For applications requiring less selectivity, less stringent hybridization conditions can be used. For example, salt conditions from about 0.14 M to about 0.9M salt, at temperatures ranging from about 20° C. to about 55° C. The presence of a hybridized complex comprising the probe or primer and a complementary polynucleotide from the test sample indicates the presence of M. tuberculosis or a M. tuberculosis polynucleotide sequence in the sample.

### Antibodies

Antibodies of the invention are antibody molecules that specifically and stably bind to a M. tuberculosis polypeptide of the invention or fragment thereof. An antibody of the invention can be a polyclonal antibody, a monoclonal antibody, a single chain antibody (scFv), or an antigen-binding portion of an antibody. An antigen-binding portion of an antibody is a part of an antibody comprising the antigen binding site or variable region of the antibody, wherein the portion is free of the constant heavy chain domains of the Fc region of the intact antibody. Examples of antigen-binding portions include Fab, Fab′, Fab′-SH, F(ab′)₂ and Fv fragments.

A purified antibody is an antibody preparation that is substantially free of cellular material, other types of antibodies, or other contaminants. An antibody preparation that is substantially free of cellular material, other antibodies or other contaminants has less than about 30%, 20%, 10%, 5%, 1% or more of other antibodies, cellular material or other contaminants. Therefore, a purified antibody is about 70%, 80%, 90%, 95%, 99% or more pure.

An antibody of the invention can be any antibody class, including for example, IgG, IgM, IgA, IgD and IgE. An antibody or antigen-binding portion thereof binds to an epitope of a polypeptide of the invention. An antibody can be made in vivo in suitable laboratory animals or in vitro using recombinant DNA techniques. Means for preparing and characterizing antibodies are well know in the art. See, e.g., Dean, Methods Mol. Biol. 80:23-37 (1998); Dean, Methods Mol. Biol. 32:361-79 (1994); Baileg, Methods Mol. Biol. 32:381-88 (1994); Gullick, Methods Mol. Biol. 32:389-99 (1994); Drenckhahn et al. Methods Cell. Biol. 37:7-56 (1993); Morrison, Ann. Rev. Immunol. 10:239-65 (1992); Wright et al. Crit. Rev. Immunol. 12:125-68 (1992). For example, polyclonal antibodies can be produced by administering a polypeptide of the invention to an animal, such as a human or other primate, mouse, rat, rabbit, guinea pig, goat, pig, dog, cow, sheep, donkey, or horse. Serum from the immunized animal is collected and the antibodies are purified from the plasma by, for example, precipitation with ammonium sulfate, followed by chromatography, such as affinity chromatography. Techniques for producing and processing polyclonal antibodies are known in the art.

“Specifically binds” or “specific for” means that a first antigen, e.g., a polypeptide, recognizes and binds to an antibody of the invention with greater affinity than to other, non-specific molecules. A non-specific molecule is an antigen that shares no common epitope with the first antigen. For example, an antibody raised against an antigen (e.g., a polypeptide) to which it binds more efficiently than to a non-specific antigen can be described as specifically binding to the antigen. In one embodiment an antibody or antigen-binding portion thereof specifically binds to a polypeptide consisting of SEQ ID NOs:1-44 when it binds with a binding affinity about Kₐ of 10⁷ l/mol or more. Specific binding can be tested using, for example, an enzyme-linked immunosorbant assay (ELISA), a radioimmunoassay (RIA), or a western blot assay using methodology well known in the art.

Additionally, monoclonal antibodies directed against epitopes present on a polypeptide of the invention can also be readily produced. For example, normal B cells from a mammal, such as a mouse, which was immunized with a polypeptide of the invention can be fused with, for example, HAT-sensitive mouse myeloma cells to produce hybridomas. Hybridomas producing M. tuberculosis-specific antibodies can be identified using RIA or ELISA and isolated by cloning in semi-solid agar or by limiting dilution. Clones producing M. tuberculosis-specific antibodies are isolated by another round of screening. Monoclonal antibodies can be screened for specificity using standard techniques, for example, by binding a polypeptide of the invention to a microtiter plate and measuring binding of the monoclonal antibody by an ELISA assay. Techniques for producing and processing monoclonal antibodies are known in the art. See e.g., Kohler & Milstein, Nature, 256:495 (1975). Particular isotypes of a monoclonal antibody can be prepared directly, by selecting from the initial fusion, or prepared secondarily, from a parental hybridoma secreting a monoclonal antibody of a different isotype by using a sib selection technique to isolate class-switch variants. See Steplewski et al., P.N.A.S. U.S.A. 82:8653 1985; Spria et al., J. Immunolog. Meth. 74:307, 1984. Monoclonal antibodies of the invention can also be recombinant monoclonal antibodies. See, e.g., U.S. Pat. No. 4,474,893; U.S. Pat. No. 4,816,567. Antibodies of the invention can also be chemically constructed. See, e.g., U.S. Pat. No. 4,676,980.

Antibodies of the invention can be chimeric (see, e.g., U.S. Pat. No. 5,482,856), humanized (see, e.g., Jones et al., Nature 321:522 (1986); Reichmann et al., Nature 332:323 (1988); Presta, Curr. Op. Struct. Biol. 2:593 (1992)), or human antibodies. Human antibodies can be made by, for example, direct immortalization, phage display, transgenic mice, or a Trimera methodology, see e.g., Reisener et al., Trends Biotechnol. 16:242-246 (1998).

Antibodies that specifically bind M. tuberculosis antigens (e.g., M. tuberculosis polypeptides) are particularly useful for detecting the presence of M. tuberculosis or M. tuberculosis antigens in a sample, such as a serum, blood, lung lavage fluid, sputum, urine or saliva sample from a M. tuberculosis-infected animal such as a human. An immunoassay for M. tuberculosis or a M. tuberculosis antigen can utilize one antibody or several antibodies. An immunoassay for M. tuberculosis or a M. tuberculosis antigen can use, for example, a monoclonal antibody directed towards a M. tuberculosis epitope, a combination of monoclonal antibodies directed towards epitopes of one M. tuberculosis polypeptide, monoclonal antibodies directed towards epitopes of different M. tuberculosis polypeptides, polyclonal antibodies directed towards the same M. tuberculosis antigen, polyclonal antibodies directed towards different M. tuberculosis antigens, or a combination of monoclonal and polyclonal antibodies. Immunoassay protocols can be based upon, for example, competition, direct reaction, or sandwich type assays using, for example, labeled antibody. Antibodies of the invention can be labeled with any type of label known in the art, including, for example, fluorescent, chemiluminescent, radioactive, enzyme, colloidal metal, radioisotope and bioluminescent labels.

Antibodies of the invention or antigen-binding portions thereof can be bound to a support and used to detect the presence of M. tuberculosis or a M. tuberculosis antigen. Supports include, for example, glass, polystyrene, polypropylene, polyethylene, dextran, nylon, amylases, natural and modified celluloses, polyacrylamides, agaroses and magletite.

Antibodies of the invention can further be used to isolate M. tuberculosis organisms or M. tuberculosis antigens by immunoaffinity columns. The antibodies can be affixed to a solid support by, for example, adsorption or by covalent linkage so that the antibodies retain their immunoselective activity. Optionally, spacer groups can be included so that the antigen binding site of the antibody remains accessible. The immobilized antibodies can then be used to bind M. tuberculosis organisms or M. tuberculosis antigens from a sample, such as a biological sample including saliva, serum, sputum, blood, lung lavage fluid, urine, feces, cerebrospinal fluid, amniotic fluid, wound exudate, or tissue. The bound M. tuberculosis organisms or M. tuberculosis antigens are recovered from the column matrix by, for example, a change in pH.

Antibodies of the invention can also be used in immunolocalization studies to analyze the presence and distribution of a polypeptide of the invention during various cellular events or physiological conditions. Antibodies can also be used to identify molecules involved in passive immunization and to identify molecules involved in the biosynthesis of non-protein antigens. Identification of such molecules can be useful in vaccine development. Antibodies of the invention, including, for example, monoclonal antibodies and single chain antibodies, can be used to monitor the course of amelioration of a disease caused by M. tuberculosis. By measuring the increase or decrease of M. tuberculosis antibodies to M. tuberculosis antigens in a test sample from an animal, it can be determined whether a particular therapeutic regiment aimed at ameliorating the disorder is effective. Antibodies can be detected and/or quantified using for example, direct binding assays such as RIA, ELISA, or western blot assays. Antibodies of the invention can be also be administered to a patient as a passive immunization therapy.

### Methods of Detection

The methods of the invention can be used to detect antibodies or antigen-binding portions thereof specific for M. tuberculosis in a test sample, such as a biological sample, an environmental sample, or a laboratory sample. A biological sample can include, for example, sera, blood, cells, plasma, lung lavage fluid, sputum, saliva, or tissue from a mammal such as a horse, cat, dog or human. The test sample can be untreated, precipitated, fractionated, separated, diluted, concentrated, or purified before combining with a polypeptide of the invention.

The methods comprise contacting a polypeptide of the invention (or a fragment thereof comprising at least one epitope) with a test sample under conditions that allow a polypeptide/antibody complex, i.e., an immunocomplex, to form. That is, a polypeptide of the invention specifically binds to an antibody or antigen-binding portion thereof specific for M. tuberculosis located in the sample. One of skill in the art is familiar with assays and conditions that are used to detect antibody/polypeptide complex binding. The formation of a complex between polypeptides and anti-M. tuberculosis antibodies in the sample is detected.

An antibody of the invention can be used in a method of the diagnosis of M. tuberculosis infection by obtaining a test sample from a human or animal suspected of having a M. tuberculosis infection. The test sample is contacted with an antibody of the invention under conditions enabling the formation of an antibody-antigen complex (i.e., an immunocomplex). The amount of antibody-antigen complexes can be determined by methodology known in the art. A level that is higher than that formed in a control sample indicates a M. tuberculosis infection. Alternatively, a polypeptide of the invention can be contacted with a test sample. M. tuberculosis antibodies in a positive body sample will form an antigen-antibody complex under suitable conditions. The amount of antibody-antigen complexes can be determined by methods known in the art. This method can indicate active infection even in cases where a patient has been vaccinated. This method can also indicate a secondary infection in patients who have had a past infection.

In one embodiment of the invention, the polypeptide/antibody complex is detected when an indicator reagent, such as an enzyme conjugate, which is bound to the antibody, catalyzes a detectable reaction. Optionally, an indicator reagent comprising a signal generating compound can be applied to the polypeptide/antibody complex under conditions that allow formation of a polypeptide/antibody/indicator complex. The polypeptide/antibody/indicator complex is detected. Optionally, the polypeptide or antibody can be labeled with an indicator reagent prior to the formation of a polypeptide/antibody complex. The method can optionally comprise a positive or negative control.

In one embodiment of the invention, antibodies of the invention are attached to a solid phase or substrate. A test sample potentially comprising a protein comprising a polypeptide of the invention is added to the substrate. Antibodies that specifically bind polypeptides of the invention are added. The antibodies can be the same antibodies used on the solid phase or can be from a different source or species and can be linked to an indicator reagent, such as an enzyme conjugate. Wash steps can be performed prior to each addition. A chromophore or enzyme substrate is added and color is allowed to develop. The color reaction is stopped and the color can be quantified using, for example, a spectrophotometer.

In another embodiment of the invention, antibodies of the invention are attached to a solid phase or substrate. A test sample potentially comprising a protein comprising a polypeptide of the invention is added to the substrate. Second anti-species antibodies that specifically bind polypeptides of the invention are added. These second antibodies are from a different species than the solid phase antibodies. Third anti-species antibodies are added that specifically bind the second antibodies and that do not specifically bind the solid phase antibodies are added. The third antibodies can comprise an indicator reagent such as an enzyme conjugate. Wash steps can be performed prior to each addition. A chromophore or enzyme substrate is added and color is allowed to develop. The color reaction is stopped and the color can be quantified using, for example, a spectrophotometer.

Assays of the invention include, but are not limited to those based on competition, direct reaction or sandwich-type assays, including, but not limited to enzyme linked immunosorbent assay (ELISA), western blot, IFA, radioimmunoassay (RIA), horizontal flow chromatography, dot blot, hemagglutination (HA), fluorescence polarization immunoassay (FPIA), and microtiter plate assays (any assay done in one or more wells of a microtiter plate).

Assays can use solid phases or substrates or can be performed by immunoprecipitation or any other methods that do not utilize solid phases. Where a solid phase or substrate is used, a polypeptide of the invention is directly or indirectly attached to a solid support or a substrate such as a microtiter well, magnetic bead, non-magnetic bead, column, matrix, membrane, fibrous mat composed of synthetic or natural fibers (e.g., glass or cellulose-based materials or thermoplastic polymers, such as, polyethylene, polypropylene, or polyester), sintered structure composed of particulate materials (e.g., glass or various thermoplastic polymers), or cast membrane film composed of nitrocellulose, nylon, polysulfone or the like (generally synthetic in nature). All of these substrate materials can be used in suitable shapes, such as films, sheets, or plates, or they may be coated onto or bonded or laminated to appropriate inert carriers, such as paper, glass, plastic films, or fabrics. Suitable methods for immobilizing peptides on solid phases include ionic, hydrophobic, covalent interactions and the like.

In one type of assay format, one or more polypeptides can be coated on a solid phase or substrate. A test sample suspected of containing an anti-M. tuberculosis antibody or antigen-binding portion thereof is incubated with an indicator reagent comprising a signal generating compound conjugated to an antibody or antigen-binding portion thereof specific for M. tuberculosis for a time and under conditions sufficient to form antigen/antibody complexes of either antibodies of the test sample to the polypeptides of the solid phase or the indicator reagent compound conjugated to an antibody specific for M. tuberculosis to the polypeptides of the solid phase. The reduction in binding of the indicator reagent conjugated to an anti-M. tuberculosis antibody to the solid phase can be quantitatively measured. A measurable reduction in the signal compared to the signal generated from a confirmed negative M. tuberculosis test sample indicates the presence of anti-M. tuberculosis antibodies in the test sample. This type of assay can quantitate the amount of anti-M. tuberculosis antibodies in a test sample. This method can indicate active infection even in cases where a patient has been vaccinated. This method can also indicate a secondary infection in patients who have had a past infection.

In another type of assay format, one or more polypeptides of the invention are immobilized onto a support or substrate. A polypeptide of the invention is conjugated to an indicator reagent and added to a test sample. This mixture is applied to the support or substrate. If M. tuberculosis-specific antibodies are present in the test sample they will bind the polypeptide conjugated to an indicator reagent and to the polypeptide immobilized on the support. The polypeptide/antibody/indicator complex can then be detected. This type of assay can quantitate the amount of anti-M. tuberculosis antibodies in a test sample.

In another type of assay format, one or more polypeptides of the invention are immobilized onto a support or substrate. The test sample is applied to the support or substrate and incubated. Unbound components from the sample are washed away by washing the solid support with a wash solution. If M. tuberculosis-specific antibodies are present in the test sample, they will bind to the polypeptide immobilized on the solid phase. This polypeptide/antibody complex can be detected using a second species-specific antibody that is conjugated to an indicator reagent. The polypeptide/antibody/anti-species antibody indicator complex can then be detected. This type of assay can quantitate the amount of anti-M. tuberculosis antibodies in a test sample.

The formation of a polypeptide/antibody complex or a polypeptide/antibody/indicator complex can be detected by radiometric, calorimetric, fluorometric, size-separation, or precipitation methods, for example. Optionally, detection of a polypeptide/antibody complex is by the addition of a secondary antibody that is coupled to an indicator reagent comprising a signal generating compound. Indicator reagents comprising signal generating compounds (labels) associated with a polypeptide/antibody complex can be detected using the methods described above and include chromogenic agents, catalysts such as enzyme conjugates fluorescent compounds such as fluorescein and rhodamine, chemiluminescent compounds such as dioxetanes, acridiniums, phenanthridiniums, ruthenium, and luminol, radioactive elements, direct visual labels, as well as cofactors, inhibitors, magnetic particles, and the like. Examples of enzyme conjugates include alkaline phosphatase, horseradish peroxidase, beta-galactosidase, and the like. The selection of a particular label is not critical, but it will be capable of producing a signal either by itself or in conjunction with one or more additional substances.

Formation of the complex is indicative of the presence of anti-M. tuberculosis antibodies in a test sample. Therefore, the methods of the invention can be used to diagnose M. tuberculosis infection in a patient. This method can indicate active infection even in cases where a patient has been vaccinated. This method can also indicate a secondary infection in patients who have had a past infection.

The methods of the invention can also indicate the amount or quantity of anti-M. tuberculosis antibodies in a test sample. With many indicator reagents, such as enzyme conjugates, the amount of antibody present is proportional to the signal generated. Depending upon the type of test sample, it can be diluted with a suitable buffer reagent, concentrated, or contacted with a solid phase without any manipulation. For example, it usually is preferred to test serum or plasma samples that previously have been diluted, or concentrate specimens such as urine, in order to determine the presence and/or amount of antibody present.

Methods of the invention can also be used to test for primary tuberculosis; to test for dormant infections; to test for secondary tuberculosis; to test for primary tuberculosis; and/or to test for all three stages of infection.

The invention further comprises assay kits for detecting anti-M. tuberculosis antibodies or antibody fragments, M. tuberculosis, or M. tuberculosis polypeptides in a sample. A kit comprises one or more polypeptides of the invention and means for determining binding of the polypeptide to anti-M. tuberculosis antibodies or antigen-binding portions thereof in the sample. A kit or article of manufacture can also comprise one or more antibodies or antigen-binding potions thereof of the invention and means for determining binding of the antibodies or antigen-binding portions thereof to M. tuberculosis or M. tuberculosis polypeptides in the sample. A kit can comprise a device containing one or more polypeptides or antibodies of the invention and instructions for use of the one or more polypeptides or antibodies for, e.g., the identification of a M. tuberculosis infection in a mammal. The kit can also comprise packaging material comprising a label that indicates that the one or more polypeptides or antibodies of the kit can be used for the identification of M. tuberculosis infection. Other components such as buffers, controls, and the like, known to those of ordinary skill in art, can be included in such test kits. The polypeptides, antibodies, assays, and kits of the invention are useful, for example, in the diagnosis of individual cases of M. tuberculosis infection in a patient, as well as epidemiological studies of M. tuberculosis outbreaks.

Polypeptides, antibodies, and assays of the invention can be combined with other polypeptides, antibodies, or assays to detect the presence of M. tuberculosis along with other organisms. For example, polypeptides and assays of the invention can be combined with reagents that detect HIV.

Methods of Treatment, Amelioration, or Prevention of a Disease Caused by M. tuberculosis

Polypeptides, polynucleotides, and antibodies of the invention can be used to immunize a subject, such as a mammal, against an M. tuberculosis infection, passively immunize a subject, stimulate a M. tuberculosis specific immunogenic response, or ameliorate or reduce the severity of one or more symptoms of a disease caused by M. tuberculosis. For example, an antibody, such as a monoclonal antibody of the invention or antigen-binding portions thereof, can be administered to an animal, such as a human. In one embodiment of the invention one or more antibodies or antigen-binding portions thereof are administered to an animal or human in a pharmaceutical composition comprising a pharmaceutically acceptable carrier. A composition comprises a therapeutically effective amount of an antibody or fragments thereof. A therapeutically effective amount is an amount effective in alleviating the symptoms of M. tuberculosis infection or in reducing the amount of M. tuberculosis organisms in a subject.

Polypeptides or polynucleotides of the invention can be present in an immunogenic composition and used to elicit an immune response in a host. An immunogenic composition is capable of inducing an immune response in an animal. An immunogenic polypeptide or polynucleotide composition of the invention is particularly useful in sensitizing an immune system of an animal such that, as one result, an immune response is produced that ameliorates or prevents the effect of M. tuberculosis infection. The elicitation of an immune response in animal model can be useful to determine, for example, optimal doses or administration routes. Elicitation of an immune response can also be used to treat, prevent, or ameliorate a disease or infection caused by M. tuberculosis. An immune response includes humoral immune responses or cell mediated immune responses, or a combination thereof. An immune response can also comprise the promotion of a generalized host response, e.g., by promoting the production of defensins.

The generation of an antibody titer by an animal against M. tuberculosis can be important in protection from infection and clearance of infection. Detection and/or quantification of antibody titers after delivery of a polypeptide or polynucleotide can be used to identify epitopes that are particularly effective at eliciting antibody titers. Epitopes responsible for a strong antibody response to M. tuberculosis can be identified by eliciting antibodies directed against M. tuberculosis polypeptides of different lengths. Antibodies elicited by a particular polypeptide epitope can then be tested using, for example, an ELISA assay to determine which polypeptides contain epitopes that are most effective at generating a strong response. Polypeptides or fusion proteins that contain these epitopes or polynucleotides encoding the epitopes can then be constructed and used to elicit a strong antibody response.

A polypeptide, polynucleotide, or antibody of the invention can be administered to a mammal, such as a mouse, rabbit, guinea pig, macaque, baboon, chimpanzee, human, cow, sheep, pig, horse, dog, cat, or to animals such as chickens or ducks, to elicit antibodies in vivo. Injection of a polynucleotide has the practical advantages of simplicity of construction and modification. Further, injection of a polynucleotide results in the synthesis of a polypeptide in the host. Thus, the polypeptide is presented to the host immune system with native post-translational modifications, structure, and conformation. A polynucleotide can be delivered to a subject as “naked DNA.”

Administration of a polynucleotide, polypeptide, or antibody can be by any means known in the art, including intramuscular, intravenous, intrapulmonary, intramuscular, intradermal, intraperitoneal, or subcutaneous injection, aerosol, intranasal, infusion pump, suppository, mucosal, topical, and oral, including injection using a biological ballistic gun (“gene gun”). A polynucleotide, polypeptide, or antibody can be accompanied by a protein carrier for oral administration. A combination of administration methods can also be used to elicit an immune response. Antibodies can be administered at a daily dose of about 0.5 mg to about 200 mg. In one embodiment of the invention antibodies are administered at a daily dose of about 20 to about 100 mg.

Pharmaceutically acceptable carriers and diluents for therapeutic use are well known in the art and are described in, for example, Remington's Pharmaceutical Sciences, Mack Publishing Co. (A. R. Gennaro ed. (1985)). The carrier should not itself induce the production of antibodies harmful to the host. Such carriers include, but are not limited to, large, slowly metabolized, macromolecules, such as proteins, polysaccharides such as latex functionalized SEPHAROSE®, agarose, cellulose, cellulose beads and the like, polylactic acids, polyglycolic acids, polymeric amino acids such as polyglutamic acid, polylysine, and the like, amino acid copolymers, peptoids, lipitoids, and inactive, avirulent virus particles or bacterial cells. Liposomes, hydrogels, cyclodextrins, biodegradable nanocapsules, and bioadhesives can also be used as a carrier for a composition of the invention.

Pharmaceutically acceptable salts can also be used in compositions of the invention, for example, mineral salts such as hydrochlorides, hydrobromides, phosphates, or sulfates, as well as salts of organic acids such as acetates, proprionates, malonates, or benzoates. Especially useful protein substrates are serum albumins, keyhole limpet hemocyanin, immunoglobulin molecules, thyroglobulin, ovalbumin, tetanus toxoid, and other proteins well known to those of skill in the art. Compositions of the invention can also contain liquids or excipients, such as water, saline, phosphate buffered saline, Ringer's solution, Hank's solution, glucose, glycerol, dextrose, malodextrin, ethanol, or the like, singly or in combination, as well as substances such as wetting agents, emulsifying agents, tonicity adjusting agents, detergent, or pH buffering agents. Additional active agents, such as bacteriocidal agents can also be used.

If desired, co-stimulatory molecules, which improve immunogen presentation to lymphocytes, such as B7-1 or B7-2, or cytokines such as MIP1α, GM-CSF, IL-2, and IL-12, can be included in a composition of the invention. Optionally, adjuvants can also be included in a composition. Adjuvants are substances that can be used to nonspecifically augment a specific immune response. Generally, an adjuvant and a polypeptide of the invention are mixed prior to presentation to the immune system, or presented separately, but are presented into the same site of the animal. Adjuvants can include, for example, oil adjuvants (e.g. Freund's complete and incomplete adjuvants) mineral salts (e.g. Alk(SO₄)₂; AlNa(SO₄)₂, AlNH₄(SO₄), Silica, Alum, Al(OH)₃, and Ca₃(PO₄)₂), polynucleotides (i.e. Polyic and Poly AU acids), and certain natural substances (e.g. wax D from Mycobacterium tuberculosis, as well as substances found in Corynebacterium parvum, Bordetella pertussis and members of the genus Brucella. Adjuvants which can be used include, but are not limited to MF59-0, aluminum hydroxide, N-acetyl-muramyl-L-threonyl-D-isoglutamine (thr-MDP), N-acetyl-nor-muramyl-L-alanyl-D-isoglutamine (CGP 11637), referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/TWEEN® 80 emulsion.

The compositions of the invention can be formulated into ingestible tablets, buccal tablets, troches, capsules, elixirs, suspensions, syrups, wafers, injectable formulations, mouthwashes, dentrifices, and the like. The percentage of one or more polypeptides, polynucleotides, or antibodies of the invention in such compositions and preparations can vary from 0.1% to 60% of the weight of the unit.

Administration of polypeptides, polynucleotides, or antibodies can elicit an immune response in the animal that lasts for at least 1 week, 1 month, 3 months, 6 months, 1 year, or longer. Optionally, an immune response can be maintained in an animal by providing one or more booster injections of the polypeptide, polynucleotide, or antibodies at 1 month, 3 months, 6 months, 1 year, or more after the primary injection. If desired, co-stimulatory molecules or adjuvants can also be provided before, after, or together with the compositions.

A composition of the invention comprising a polypeptide, polynucleotide, antibody, or a combination thereof is administered in a manner compatible with the particular composition used and in an amount that is effective to elicit an immune response as detected by, for example, an ELISA. A polynucleotide can be injected intramuscularly to a mammal, such as a baboon, chimpanzee, dog, or human, at a dose of 1 ng/kg, 10 ng/kg, 100 ng/kg, 1000 ng/kg, 0.001 mg/kg, 0.1 mg/kg, or 0.5 mg/kg. A polypeptide or antibody can be injected intramuscularly to a mammal at a dose of 0.01, 0.05, 0.5, 0.75, 1.0, 1.5, 2.0, 2.5, 5 or 10 mg/kg.

Polypeptides, polynucleotides, or antibodies, or a combination thereof can be administered either to an animal that is not infected with M. tuberculosis or can be administered to a M. tuberculosis-infected animal. The particular dosages of polynucleotide, polypeptides, or antibodies in a composition will depend on many factors including, but not limited to the species, age, gender, concurrent medication, general condition of the mammal to which the composition is administered, and the mode of administration of the composition. An effective amount of the composition of the invention can be readily determined using only routine experimentation.

Additionally, the invention provides for methods of determining effectiveness of a treatment for a M. tuberculosis. For example, an amount or presence of one or more polypeptides comprising SEQ ID NOs:1-44 can be determined in a test sample from a patient infected with M. tuberculosis prior to treatment. The amount or presence of the one or more polypeptides comprising SEQ ID NOs:1-44 in a test sample can then be determined after one or more treatments. The amount or presence of the one or more polypeptides before and after treatment can be compared. A lesser amount of the polypeptides after treatment as compared to before treatment indicates effectiveness of the treatment; and the absence of the polypeptides in after treatment as compared to presence of the polypeptides in before treatment indicates effectiveness of the treatment.

Another method of determining effectiveness of a treatment for a M. tuberculosis infection comprises determining an amount of one or more polypeptides comprising SEQ ID NOs:1-44 in a test sample from a patient infected with M. tuberculosis. The amount of the one or more polypeptides comprising SEQ ID NOs:1-44 is compared to a standard. The standard can comprise, e.g., no M. tuberculosis polypeptides of SEQ ID NO:1-44. If the amount of the one or more polypeptides is greater than the standard, then the treatment is ineffective. If the amount of the one or more polypeptides is less than or equal to the standard then the treatment is effective.

The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their customary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above. All references cited in this disclosure are incorporated herein by reference.

### EXAMPLES

### Example 1

### Genomic Expression Library Construction

The IVIAT scheme shown in FIG. 1 depicts the 7 basic steps required to isolate and identify IVI antigens of a human microbial pathogen. The choice of an appropriate starting strain for IVIAT-based identification of IVI genes of a pathogen is more or less critical depending on the ultimate goals of the project. In instances where the main thrust of the work is directed toward identifying in vivo expressed proteins that are likely candidates for diagnostic or vaccine strategies, an antigen common to all pathogenic clonotypes is desired. In this case, it is reasonable to construct the genomic expression library using DNA from a single, pathogenic strain. In instances where the main thrust of the work is directed toward obtaining the broadest possible understanding of the pathogenic mechanisms used by the microorganism, we typically use one or more representatives of each clonotype to maximize the chances of identifying the broadest array of virulence genes. It is clear that there is significant genetic diversity within the species M. tuberculosis (Burman et al., 1997; Filliol et al., 1999; Kato-Maeda et al., 2001), but since we are primarily interested in diagnostic applications, we constructed our library using CSU93 DNA provided by Colorado State University. This strain is suitable for IVIAT analysis since it is a recent clinical isolate from the Kentucky/Tennessee region (Valway et al., 1998) and its 4.4 Mb genome has been sequenced by TIGR. It would be preferable to start with cells and DNA from a multidrug resistant strain, but these are not currently available through CSU. CSU93 is pan-drug sensitive.

Library construction was performed using the pET30abc system of Novagen (Madison, Wis.). There are several important characteristics of this vector system that make it desirable for use in IVIAT. First, the pET system allows transcription of cloned DNA by T7 RNA polymerase (DE3) at a T7 promoter when the construct is in an appropriate lambda lysogen host such as BL21(DE3). In this host, potential toxic effects of cloned genes are prevented by the presence of a lac operator that permits tight regulation of expression by IPTG. Second, the plasmids provide a ribosome binding site (RBS) and a (histidine)₆-tag (His-tag) immediately following the translation start codon just upstream of a multiple cloning site. The His-tag potentially allows rapid purification of translated proteins using nickel affinity chromatography and provides a convenient marker in Western blot analyses. Finally, the system has specifically been engineered to allow universal in-frame cloning by introducing one (pET30b) and two (pET30c) base deletions before the multiple cloning site in pET30a.

We obtained genomic DNA from strain CSU93 (CDC 1551) from Colorado State University. A 10 μg sample of the DNA was dissolved in TE buffer and treated in a Hydroshear (Genomic Solutions, Ann Arbor, Mich.) under conditions that optimized production of 0.5-1.5 Kb fragments. This approach avoids the bias created by using restriction enzymes. Fragments of this size were cut from a 0.75% agarose gel and purified using GeneClean Turbo (Q-BIOgene, Carlsbad, Calif.). Terminal overhangs were removed using the END-IT™ DNA end-repair kit from Epicenter (Madison, Wis.) and the resulting blunt end products were ligated into the CIP-dephosphorylated EcoRV restriction site in the pET30c multiple cloning site. The vector library was amplified by electroporating it into E. coli NOVABLUE™ cells (Novagen) with transformants selected on BHI/kanamycin medium. A minimum of 10⁵ independent Top10 clones were created. Colonies that arose were collected, washed in PBS, and plasmid DNA isolated using the WIZARD® Plus maxiprep (Promega, Madison, Wis.). The purified plasmid DNA was used to transform chemically competent BL21(DE3), again with selection on BHI/kanamycin plates. Crack preparations of 100 clones were examined by agarose gel electrophoresis to provide assurance that the library contained a high (>95%) proportion of inserts in pET30 of various sizes ranging from 0.5 to 1.5 Kb. Sterile 80% glycerol was added to the library to give a final concentration of 30% (v/v), and aliquots were stored frozen at −80° C. until used in the screening step.

### Example 2

### Antibody Probe Preparation

Serum was obtained from the World Health Organization with sufficient accompanying information to stage the infection and determine its severity. Ten sera were selected representing primary infections (4), recurrent infections (4) and dormant infections (2). Each subgroup of sera spanned a significant timeframe, from weeks to months following the onset of the particular stage. In this fashion, the possibility of identifying transiently expressed genes was enhanced. Before adding to the pool, each serum was tested individually to verify high titer, broad reactivity to CSU93 extracts (provided by Colorado State University) by a modified ELISA procedure (Ebersole 1980) and by Western blots. Equal volumes of individual serum samples were then pooled. The pooled human serum was adsorbed to remove antibodies that were reactive with proteins made by CSU93 during in vitro cultivation. To accomplish this, 500 μl of the serum was subjected to five rounds of adsorption against whole, irradiated cells of CSU93 (FIG. 2). Each adsorption consisted of a 2 hour incubation with mild agitation at 4° C. of the pooled serum with approximately 10¹⁰ cells in 100 μl phosphate buffered saline (PBS) containing 0.02% sodium azide. The serum was then adsorbed with cell extracts of CSU93 bound to 0.49 μm latex beads (Bangs Laboratories, Fishers, Ind.) according to the manufacturer's instructions. This step was repeated using heat denatured CSU93 cell lysates prepared by incubation in a boiling water bath for 10 minutes. Then, the serum was treated with a lysate of IPTG-induced BL21 (DE3)/pET30b cells bound to latex beads. Small (10 μl) samples were taken after each adsorption step and tested by ELISA and Western blot analysis to verify the completeness of the process. A titer of <1:100 in ELISA and no visible reactivity in Western blots using a 1:100 dilution of primary antibody served as the endpoint criteria for the adsorption process. Unadsorbed serum and secondary serum alone served as positive and negative controls for the ELISA and Western studies. Dot blot analysis using goat anti-human IgG showed no significant decrease in the overall amount of IgG of the adsorbed serum, indicating that the adsorption process was specific and that no non-specific protease-mediated degradation or immunoglobulin binding had occurred. IgG was purified from the adsorbed serum using a protein A column and the BioLogic Duo-Flow Protein Purification System (BioRad, Hercules, Calif.). The final antibody probe was aliquoted and stored frozen at −80° C. until used.

### Example 3

### Probing the Expression Library

Samples of the genomic expression library were thawed and diluted, and samples spread to BHI/kan plates to give approximately 500 colonies per plate. The colonies were lifted onto PROTRAN® nitrocellulose membranes (0.2 μm; Schleicher & Schuell, Keene, N.H.) and placed, colony side up, onto BHI/kan medium containing 1 mM IPTG. The plates were incubated for 3 hours at 37° C. to induce expression of cloned genes. The original library plates were reincubated for 3 to 5 hours to regrow the colonies and served as the “master plates” for later use in isolating reactive clones. The membranes were then placed on Whatman #1 filter papers saturated with chloroform for 10 seconds to lyse cells and thereby liberate expressed proteins. The membranes were air dried and blocked for 1 hour at room temperature in PBS-TWEEN™ containing 5% non-fat skim milk. Following 3 successive washes in PBS/TWEEN™, the membranes were incubated overnight at 4° C. with gentle rocking with the IVI antibody probe prepared in step 2 and diluted 1:2500 in PBS-TWEEN™. Next, the membranes were washed 3 times with PBS/TWEEN™ and then incubated with the secondary antibody, affinity purified peroxidase-conjugated goat anti-human immunoglobulin (ICN/Cappel) diluted 1:20.000 in PBS/TWEEN™ for 1 hour at room temperature with gentle rocking. The membranes were washed 3 times with PBS-TWEEN™ and twice with PBS. Reactive clones were visualized by the addition of SUPERSIGNAL® West Femto Maximum Sensitivity Substrate (Pierce, Rockford, Ill.) and exposure for 15, 30, 45, 60, 75 and 90 second intervals on a UVP chemiluminescence imager. A size adjusted picture from the imager was used to identify reactive clones on the master plates, which were picked and purified by streaking on selective medium. The adsorption process eliminated a large number of colonies reactive with unadsorbed serum which, presumably, are ones that are producing proteins made by in vitro grown cells.

Our calculations indicated that approximately 125,000 independent clones with an average insert size of 1 kb would provide 99% coverage for the M. tuberculosis 4.4 Mb genome taking into account 2 orientations of insertion and 3 different reading frames. Assuming a maximum of 3 ORFs on an insert this size, we would require 500,000 clones to assure blanket coverage of the CSU93 chromosome. We did screen this number of clones over a 3 month period.

### Example 4

### Secondary Screening

To reduce the number of false positives, clones isolated and purified from the primary screening were retested for reactivity with the adsorbed serum. The clones were grown overnight at 37° C. in 1 ml of BHI broth containing kanamycin, and the cells were pelleted and resuspended in a minimum volume of fresh medium. After resuspension, 5 μL were spotted onto BHI agar plates containing kanamycin with or without IPTG (1 mM). Following incubation of the plates at 37° C. for five hours, clones were treated with chloroform vapors and proteins were immobilized on nitrocellulose membranes, which were probed with adsorbed sera as described above. Two negative controls were included on each plate: pET30b/BL21(DE3) with no cloned insert, and a random clone that contained an insert but was non-reactive with adsorbed sera. After preliminary screening identified a clone expressing a highly reactive antigen, this clone was included on each plate as a positive control. Rescreening was performed at least three times for each clone to confirm reactivity. From a total of 500,000 independent clones originally screened, 116 IPTG inducible clones with medium to strong reactivity were chosen for further study. Clones that gave weak reactivity or inconsistent results in the secondary screening were stored frozen in glycerol for later investigation.

### Example 5

### Identification of ORFs Expressing Reactive Antigens

Recombinant plasmids from IPTG-inducible positive clones were purified from 1 ml BHI broth/kan cultures (Qiagen miniprep), and the cloned DNA inserts were sequenced in both directions using pET30 primers (Novagen). Sequencing was performed by the University of Florida Interdisciplinary Biotechnology Core. Typically, about 500-800 bp of good sequence was obtained from each reaction. The bidirectional sequencing data revealed the start and end points of the cloned insert, which was matched to the CSU93 genomic database to determine the overall size of the insert plus annotated ORFs. Clones containing duplicate and overlapping inserts were culled, leaving 44 unique open reading frames. The occurrence of redundant clones provided good evidence for the completeness of the primary screening performed in step 3. The sequence data were analyzed at the junction with the pET30 vector to confirm that the cloned insert was in the proper orientation and in the proper reading frame to create a fusion protein with the pET30 leader. Information concerning function, localization, structural motifs, etc. of the expressed protein were recorded in a database for easy reference.

As in the case of Candida albicans (Cheng et al., 2003) and certain other instances, most of the clones contained a fragment of only one ORF on the positive strand. In these instances, and in instances where more than one ORF was present on the positive strand, the entire ORF(s) was amplified using CSU93 DNA as template. The resulting fragment was isolated using agarose gel electrophoresis and cloned into the pET30 Ek/LIC vector system in-frame with the plasmid leader peptide using the one step protocol provided by the manufacturer (Novagen). Because BL21(DE3) is relatively poorly transformable, the recombinant construct was first transformed into NovaBlue to amplify it, and then transformed into BL21(DE3). The reactivity of the subclones with the adsorbed antibody probe was analyzed using the secondary screening method described above.

### Example 6

### PAGE and Western Blot Confirmation

Subclones have been analyzed by PAGE and Western blots to confirm that they produce an IPTG-inducible protein of the approximate size deduced from the DNA sequence, and that this band is reactive with an anti-(His-tag) antibody (the Ek/LIC leader sequence provides a his tag) and with the IVIAT antibody probe. First, conditions for overexpression of the cloned gene were determined. Replicate 1 ml aliquots of 3 different media were inoculated with the clone and grown at 37° C. overnight. The cells were then pelleted and resuspended in the same volume of medium with or without IPTG at 10, 25 and 37° C. for 1, 2 or 3 hours. The cells were collected by centrifugation and taken up in 100 μl of SDS-PAGE sample buffer containing 6% (v/v) 2-mercaptoethanol, treated by incubation for 10 minutes in a boiling water bath, and centrifuged for 5 minutes in a microfuge at top speed to remove insoluble material. Twenty-five μl samples were electrophoresed on 12% SDS-PAGE gels with standard prestained molecular weight markers. The gels were stained with Coomassie blue R250, and surveyed for conditions that yield the maximum production of IPTG inducible cloned gene product. Cell samples from the optimal production conditions were run on triplicate gels, one of which is stained with Coomassie blue and the other 2 were electroblotted onto nitrocellulose membranes (Schleicher & Schuell) using blot buffer at 400 mA (constant amperage) for 1 hour in a Hoefer Transphor (Hoefer Scientific Instruments, San Francisco). One of these blots were probed with peroxidase-conjugated mouse anti-(His)₆ IgG1 monoclonal antibody (Cappel/ICN) to confirm that the expressed IVI antigen was labeled with the His-tag. The second blot was incubated with the adsorbed antibody probe and developed as described above. The blots and the stained gel are compared to identify clones that produce an IPTG-inducible band that co-migrates with the band that is reactive with both the anti-His-tag antibody and the IVI antibody probe. Of the 44 IVI genes that were verified by the secondary screening in step 4, approximately half have gone through steps 5 and 6.

The entire list of IVI genes and the extent of their characterization is presented in Appendix 1. The sequence of the genes is publicly available and information regarding the protein product available online (e.g., GenBank). The majority of genes isolated have unknown function (19 had unknown function; 9 were membrane/surface localized, 12 were related to metabolism, 2 were related to regulatory functions, 2 were related to drug resistance). Most have a presumptive cytoplasmic localization, which serves to substantiate our statement that not only surface exposed molecules will elicit a humoral immune response. There are at least 9 that may be membrane associated, and which may therefore make interesting potential targets for vaccine approaches. Five genes are likely to be involved in gene regulation and 4 are stress associated, based on BLAST analysis. It is noteworthy that in several cases, independently isolated IVI genes are either adjacent loci or parts of known operons.

### Example 7

### Purification of the Overexpressed IVI Protein and Testing for Differential Reactivity with Patient and Control Sera

Depending on how well an IVI gene is expressed when the clone is induced with IPTG under optimal conditions of medium and time, 1 to 100 ml cultures of the clone were grown. The cells were collected by centrifugation and lysed on ice by sonication using 5×10 second bursts at resonance frequency with 30 second rest intervals to prevent overheating. The sample is centrifuged and samples of the cell free supernatant and the cell debris were examined by SDS-PAGE to determine the localization of the overexpressed IVI protein. If present in the supernatant, the protein is purified via its his tag using nickel chromatography according to the manufacturer's (Novagen) instructions. In instances where the IVI protein is present in the pellet, it is purified from inclusion bodies by washing the pellet in inclusion body buffer (20 mM Tris, pH8, 0.2 M NaCl and 1% deoxycholate) and stirring for 20 minutes at room temperature. The suspension was centrifuged and the pellet is further washed 3 times by centrifugation in inclusion body buffer 2 (10 mM Tris, pH8, 0.25% sodium deoxycholate, 1 mM EGTA) for 30 minutes at room temperature. The pellet was dissolved in a solution containing 8M urea, 0.1 mM sodium azide, 1 mM EGTA, and Tris, pH8. After thorough vortexing, an equal volume of water was added and the resulting solution was dialyzed against Tris, pH8 plus 0.8% NaCl. If the protein precipitates from solution during the dialysis, it is collected by centrifugation and stored. It does not matter if the protein is in solution or not, since the next step in the analysis is to add SDS-PAGE sample buffer and place it in a boiling water bath for 5 minutes. Samples were run on 12% SDS-PAGE gels and stained with Coomassie blue R250. In instances where the protein of interest represent >90% of the protein visible on the Coomassie stained gel, Western blots were performed using the peroxidase-conjugated mouse anti-(His)₆ IgG1 monoclonal antibody (Cappel/ICN), which may indicate if any of the minor bands are breakdown products, and the adsorbed IVI antibody probe to verify that the correct protein has been purified.

To this point, 10 IVI genes have been overexpressed and their proteins purified. An additional 10 proteins are in the final stages of purification. We have done some preliminary screening using 1 of these proteins selected at random to determine if it is reactive with serum from tuberculosis patients and not with serum from control subjects. Since the degree of purity cannot be firmly fixed for this protein, we have performed Western blot analysis to provide a clear indication of the reactivity instead of dot blots or ELISAs. Purified IVI protein derived from IVI clone 141 expressing the gi13882534 protein was analyzed with a Western blot. Obviously, no firm conclusions can be drawn from such a small sample, but it appears that the protein expressed by gi13882534 reacts more strongly with sera from active tuberculosis patients and the reactivity with sera from patients with dormant infections are intermediate. These preliminary results suggest the possibility that the protein expressed by gi13882534 could form the basis for a host immune response-dependent serological test for active tuberculosis.

### Example 8

### Western Blot Validation of IVI Genes and Purification of the Expressed Proteins

The methods developed to date for subcloning the IVI ORFs, overexpressing the cloned gene and verifying the reactivity of the expressed product with adsorbed antibody probe using Western blotting methods are described in detail above. They have been optimized and are well suited to completing the task of verifying that the cloned gene expresses a protein that is inducible with IPTG, and the expressed protein reacts with both an anti-his antibody and the adsorbed IVI antibody probe to verify that we have cloned the correct gene. The entire 12.45 Kb PCR fragment for the gi13881781 IVI gene has been cloned into Ek/LIC. We are currently beginning studies to express its 431.6 KDa protein.

The conditions established for overexpressing the cloned IVI gene during the verification step will be scaled up for the purification step as described above. Again, since downstream methods do not depend on obtaining the purified protein in a soluble form, either nickel chromatography can be used to purify the proteins via their his tag if the protein is recovered from the host strain in the soluble fraction or inclusion body preps can be performed in cases where the protein is recovered from the host strain in the insoluble fraction. In either instance, the extent of purification will be approximated by SDS-PAGE, and the protein aliquoted and stored at −80° C. until used.

In any instances where varying the cultivation media and temperature of incubation for a clone do not result in significant overproduction of the gene product, the gene can be subcloned into another vector and host. There are a number of restriction enzyme sites on both sides of the ORF cloned into Ek/LIC that should help to simplify this step. Attention will be paid to finding a high expression, regulatable expression vector, such as pRSET (Invitrogen), and installing the ORF in the proper orientation and in-frame with any leader sequence to assure the product is properly expressed.

### Example 9

### Screening of IVI Gene Products for Their Potential Use in Diagnosis of Tuberculosis

Each of the purified proteins obtained will be initially screened by Western blot using sera from healthy control subjects, healthy control subjects previously vaccinated with BCG, active primary tuberculosis patients, patients with dormant infections, and patients with secondary tuberculosis. Sera diluted 1:500 in PBS-TWEEN® can be used as described above with horseradish peroxidase conjugated goat anti-human antibody to develop the blot, which will be used at a 1:20,000 dilution. The blot will be surveyed by eye to determine the candidate proteins that are likely to be best suited for use in a host immune response-dependent serological test.

It is reasonable to assume that M. tuberculosis engaged in a primary infection expresses proteins that are not made during latency and, possibly, during secondary infections. Likewise, it is reasonable to assume that M. tuberculosis makes proteins during latency that are not made during active infections. These proteins may be made in very small amounts given the low metabolic state of the organism during latency, but if one or more or these proteins is highly immunogenic, it may be present on our list of IVI genes. A significant percentage of individuals who have been infected by M. tuberculosis have not been diagnosed as such. Thus, a test that identifies patients with dormant infections is of interest, since it would alert physicians to maintain a careful watch on them. The Western blots will be surveyed keeping in mind the various possibilities: a test specific for primary tuberculosis; a test specific for dormant infections; a test specific for secondary tuberculosis; a test that is specific for primary or secondary tuberculosis; a test that recognizes all three stages.

Proteins that demonstrate potential in any one of these regards will be further tested using a battery of sera representing primary, secondary and dormant infections. An equal number of control sera, comprised of equal numbers of BCG vaccinated and non-vaccinated healthy subjects will be tested. Since the number of individual tests is high, it will be cost and time effective to purify the test protein essentially to homogeneity so it can be used in an ELISA format rather than a Western blot format. Purification will be attempted initially using C18 reverse phase HPLC chromatography (Bio-Rad, Hercules, Calif.). A sample of the nickel column or inclusion body purified protein will be adjusted to 20% acetonitrile in the case of soluble proteins, or the minimal acetonitrile: water ratio will be used to dissolve IVI protein precipitates. Approximately 500 μg of protein will be run on an analytical C18 column using a 20 to 80% acetonitrile gradient with 0.1% trifluoracetic acid. The optical density at 214 and 280 nm will be monitored and peaks collected. Samples of the peaks will be lyophilized to remove acetonitrile and taken up in SDS-PAGE sample buffer. The samples will be run on gels to determine the peak that contains the IVI protein and to assess the degree of its purity. Large scale preparations of IVI proteins showing >99% purity will be achieved using the same conditions on a semi-preparative C18 column using 20 mg of starting protein. If this degree of purity is not achieved using C18, we will test the ability of C8 and C12 columns to achieve this goal. Purified proteins will be lyophilized and stored at 4° C. until used in an ELISA method (Ebersole et al., 1980). Briefly, the protein dissolved in binding buffer will be used to coat a polyvinyl chloride microtiter well plate for one hour at room temperature. Unbound antigen will be removed by repeated washing using a plate washer (Bio-Rad), and the wells blocked with PBS-TWEEN®. One hundred μl of each serum sample, diluted 1:500, will be added to the plate and incubated overnight at 4° C. The wells will be extensively washed and a secondary antibody, HRP-conjugated goat anti-human IgG will be added and incubated at room temperature for 1 hour. After extensive washing, ABTS substrate (BD Biosciences, Canada) containing 3% hydrogen peroxide will be added and the plates read at 10 minute intervals 405 nm between 5 and 80 minutes or until the positive control reaches an OD=1.5. Positive controls will consist of wells containing adsorbed IVI antibody probe and negative controls will lack protein or primary antibody. The data will be grouped into uninfected and infected categories and the latter will be subdivided into primary, dormant or secondary. The results will be averaged within each group and statistically significant differences will be determined two ways. First, mean data for the uninfected (control) group will be compared to an average of the three infected groups using an independent samples t test to determine overall differences between the treatment and control. Secondly, to gain a more refined understanding of differences among the infected groups a one-way analysis of variance (ANOVA) will be employed. Significant group differences will be decomposed using planned comparison t tests to determine whether the various infected groups differ from the uninfected control group.

Proteins showing promise will be used in field tests for the identification of individuals infected with M. tuberculosis. The specificity and sensitivity of the test(s) can be determined.

### Example 10

### Raising Polyclonal Antibodies Against IVI Proteins for Identification of Proteins Shed by M. tuberculosis During Active Infection

Diagnosis of tuberculosis is often difficult using available methods. Skin reactivity to PPD is an important diagnostic tool, but is ineffective in BCG vaccinated subjects and it requires an intact host immune system. Tuberculin anergy occurs in 15-25% of non-HIV infected tuberculosis patients and is close to 50% in tuberculosis patients with HIV infection. Bacteriological culture of sputum is slow and uncertain, and identification of acid fast bacilli in sputum lacks sensitivity. Molecular methods for diagnosis, such as nucleic acid amplification are relatively fast and sensitive, but they are expensive and technically complex, and therefore require a high degree of quality control for accurate performance. Tests dependent on circulating antibodies against M. tuberculosis are easy and cost efficient, but obviously are of limited use in HIV-infected subjects. Landowski et al. (2001) provided evidence that a protein complex, Ag85, known to be one of the major shed protein produced by M. tuberculosis during growth in vitro is frequently present in the blood of infected patients. Monoclonal antibodies directed against epitopes of this complex served as the basis of a test to determine active infection by looking for the shed complex in patients' blood. The results indicated that this is a viable approach, but would require the addition of additional components in order to increase the sensitivity and specificity of the test to acceptable levels. Recognizing the potential problems associated with identifying a host immune response-dependent test as described above we will also adapt our methods to looks for complementary epitopes on shed M. tuberculosis proteins that can serve as targets for a host immune response-independent test for active infection. We have devised a novel approach to achieve this end.

First, we have taken into account the possibility that circulating antigens derived from M. tuberculosis during active infection do not necessarily depend on their transport and excretion from the bacterial cell. During the course of infection, particularly as the cell mass grows, death of cell will lead to lysis and the release of breakdown products that can come from any compartment of the bacterial cell. Thus, we will take a completely undirected approach by using all of the protein products expressed by IVI genes as potential targets. We will use the following methodology:
Each purified IVI protein from will be used to immunize Sprague Dawley rats; Ribi adjuvant will be employed to boost the immune response, and the time and amount of immunogen will follow the manufacturer's (Corixa, Hamilton, Mont.) directions; sample bleeds at indicated time points will be compared to prebleed control samples using ELISA to determine when a high titer response to the immunizing protein has occurred; the serum will be collected by cardiac puncture and the sera will be pooled and stored at −80° C. until used.
Antibodies in the rat sera that can potentially react with components in normal human blood will be removed using an adsorption process similar to that described for IVIAT; pooled sera from 50 healthy subjects will be bound to latex beads (Bangs Laboratories) and used to adsorb the rat sera by repeated exposure at 4° C. with gentle mixing (this reagent can be made and stored in large amounts so that one or two batches will suffice for the treatment of all the rat sera to be tested); the reactivity of the mouse sera with the healthy human sera will be followed throughout the course of the adsorption by testing samples in an ELISA format; we expect to see a pattern similar to that shown in FIG. 2, although the initial titers may be considerably lower at the start of the experiment; the IgG will then be purified from the adsorbed rat serum using a protein A column and reverse phase HPLC.
Serum from healthy and tuberculosis patients will be used to coat the wells of a PVC microtiter plate overnight at 4° C. (The serum from healthy subjects will be different from the samples used to for the adsorption process); the plate will be blocked with PBS-TWEEN® and purified IgG from an immunized rat will be diluted 1:1000 in binding buffer and 100 μl added to each well; the plates will be incubated overnight at 4° C. and then washed thoroughly with PBS-TWEEN®; the ELISA will be developed using horse radish peroxidase conjugated donkey anti-rat IgG preadsorbed with rat serum (RDI research Diagnostics, Concord, Mass.); the ELISA will be developed using ABTS substrate (BD Biosciences) containing 3% hydrogen peroxide and the plates read at 10 minute intervals 405 nm between 5 and 80 minutes or until the positive control reaches an OD=1.5. The positive control will be wells initially coated with the purified protein and the negative control wells will not be coated initially with human serum.
The data will be analyzed to identify IVI proteins that are consistently present in sera from tuberculosis patients that are not present in sera from healthy control patients; the mean recorded value of the wells and their variance will be calculated for the two groups and compared using a student's T test; instances in which serum from tuberculosis patients is found to have significantly greater reactivity than control serum from control subjects will be identified and used to develop field tests for the identification of individuals infected with M. tuberculosis.

Landowski et al. (2001) chose to use a dot immunoblotting method to test the sensitivity and specificity of Ag85 and other proteins known to be shed during in vitro cultivation. While their values were not significantly different from host immune response-dependent tests, they note that the addition of more epitopes to the test could very likely increase the sensitivity without significantly decreasing the specificity. They chose to work with small peptide epitopes to limit the amount of cross-reactivity that may arise from exposure to saprophytic mycobacteria or from other causes. This appeared to be effective to a large extent, but clearly was not perfect. We believe that adsorption with a large number of sera from healthy individuals will remove cross-reacting antibodies more efficiently and represents the novel component of this approach. It logically should allow us to use the entire IVI protein as the target for the assay, but we observe significant background in samples treated with control sera, it would suggest that adsorptions with even larger number of normal sera is required or that using small fragments of the protein would be better suited to serve as the immunogen. In the latter case we would use the methods described by Landowski et al. (2001) to retest the IVI proteins that looked the best in the initial trial. It is also worthwhile to note that Wallis et al. (2001) used Ag85 in sputum as a surrogate marker for the efficacy of treatment of tuberculosis. It therefore would be conceivable to perform the experiments described in this application with sputum samples rather than serum in cases of pulmonary tuberculosis. There is an excellent likelihood that the strength of the reaction would be enhanced owing to the concentration effect achieved by using the primary organ affected.

### Example 11

### Host Immune Response-Independent Diagnosis

A host immune response-independent test can also be made with the polypeptides of the invention. Shed proteins of M. tuberculosis can be identified in the blood of actively infected tuberculosis subjects. To accomplish this, each purified protein (SEQ ID NO:1-44) will be used to raise an immune serum in rats. The immune serum will be exhaustively adsorbed with serum from healthy humans to remove cross reacting antibodies and the rat IgG purified to serve as a probe for shed proteins. Serum from tuberculosis and healthy subjects will be bound to microtiter wells and probed in an ELISA assay using the adsorbed rat IgG probe. Wells showing reactivity will be shown to contain M. tuberculosis shed proteins that are recognized by the immune rat IgG probe. The ability of the shed protein to serve as a marker for active infection will be determined using statistical methods.

### REFERENCES CITED

Aitchison, E. J., Lambert, P. A., Smith, E. G. and Farrell, I. D. 1987, Serodiagnosis of Streptococcus faecalis endocarditis by immunoblotting of surface protein antigens. J. Clin. Microbiol. 25:211-215.
Balasubramanian, V., Wiegeshaus, E. H., and Smith, D. W. 1994. Mycobacterial infection in guinea pigs. Immunol. 191:395-401.
Behr., M. A. and Small, P. M. 1997. Has BCG attenuated to impotence? Nature 389:133-134.
Bina, J., J. Zhu, M. Dziejman, S. Faruque, S. Calderwood, and J. Mekalanos. 2003. ToxR regulon of Vibrio cholerae and its expression in vibrios shed by cholera patients. Proc. Natl. Acad. Sci. USA 100:2801-6.
Bloom, B. R. and Small, P. M. 1998. The evolving relation between humans and Mycobacterium tuberculosis. N. Engl. J. Med. 338:677-678.
Burman, W. J., Reves, R. R., Hawkes, A. P., Rietmeijer, C. A., Yang, Z., el-Jajj, H., Bates, J. H. and Cave, M. D. 1997. DNA fingerprinting with two probes decreases clustering of Mycobacterium tuberculosis. Am. J. Respir. Crit. Care Med. 155:1140-1146.
Camacho, L. R., Ensergueix, D., Perez, E., Gicquel, B. and Guilhot, C. 1999. Identification of a virulence gene cluster of Mycobacterium tuberculosis by signature-tagged transposon mutagenesis. Mol. Microbiol. 34:257-267.
Cao, S. L., Progulske-Fox, A., Hillman, J. D. and Handfield, M. 2004. In vivo induced antigenic determinants of Actinobacillus actinomycetemcomitans. FEMS Microbiol. Lett. 237:97-103.
Casanova, J.-L. and Abel, L. 2002. Genetic dissection of immunity to mycobacteria: the human model. Ann. Rev. Immunol. 20:581-620.
Casadevall, A. and Pirofski, L. A. 2000. Host-pathogen interactions: basic concepts of microbial commensalism, colonization, infection and disease. Infect. Immun. 68:6511-6518.
Chan, E. D., Heifets, L. and Iseman, M. D. 2000. Immunologic diagnosis of tuberculosis: a review. Tuberculosis Lung Dis. 80:131-140.
Cheng, S., Clancy, C. J., Checkley, M. A., Handfield, M., Hillman, J. D., Progulske-Fox, A., Lewin, A. S., Fidel, P. L. and Nguyen, M. H. 2003. Identification of Candida albicans genes induced during thrush offers insight into pathogenesis. J. Mol. Microbiol. 48:1275-1288.
Cheng, S., Nguyen, M. H., Zhang, Z., Jia, H., Handfield, M., Hillman, J. D., Progulske-Fox, A. and Clancy, C. J. Evaluating the roles of NOT5, NOT3, BUR2, and KEL1 in Candida albicans virulence using gene disruption strains that express URA3 from the native locus. J. Mol. Microbiol., Submitted.
Colditz, G. A., Berkey, C. S. Mosteller, F., Brewer, T. F., Wilson, M. E., Burdick, E. and Fineberg, H. V. 1995. The efficacy of bacillus Calmette-Guerin vaccination of newborns and infants in the prevention of tuberculosis: meta-analyses of the published literature. Pediatrics. 96:29-35.
Cotter, P. A. and Miller, J. F. 1998. In vivo and ex vivo regulation of bacterial virulence gene expression. Curr. Opin. Microbiol. 1:17-26.
Cox, J. S., Chen, B., McNeil, M. and Jacobs, W. R., Jr. 1999. Complex lipid determines tissue-specific replication of Mycobacterium tuberculosis in mice. Nature 402:79-83.
Daniel, T. M. and Janicki, B. W. 1978. Micobacterial Antigens—Review of their isolation, chemistry, and immunological properties. Microbiol. Rev. 42:84-113
Ebersole, J. L., Frey, D. E., Taubman, M. A., and Smith, D. J. 1980. An ELISA for measuring serum antibodies to Actinobacillus actinomycetemcomitans. J. Periodont. Res. 15:621-632.
Filliol, I., Sola, C., Legrand, E. and Rastogi, N. 1999. Genotypic diversity of Mycobacterium tuberculosis in the Guiana-Antilles-Guyane. Bull. Soc. Pathol. Exot. 92:292-298.
Finlay, B. B., and Falkow, S. 1997. Common themes in microbial pathogenicity. Microbiol. Rev. 53:210-230.
Gennaro, M. L. 2001. Immunologic diagnosis of tuberculosis. Clin. Infect. Dis. 30 Suppl 3:S243-6.
Handfield, M., and Levesque, R. C. 1999. Strategies for isolation of in vivo expressed genes from bacteria. FEMS Microbiol. Rev. 23:69-91.
Handfield, M., Brady, L. J., Progulske-Fox, A., and J. D. Hillman. 2000. IVIAT: a novel method to identify bacterial genes expressed specifically in human infections. Trends Microbiol. 8:336-339.
Handfield, M., Cao, S., Progulske-Fox, A., and J. D. Hillman. IVIAT Screening of the Entire Actinobacillus actinomycetemcomitans HK1651 Genome for In Vivo Induced Genes., J. Den. Res. Submitted
Hang, L., John, M., Asaduzzaman, M., Bridges, E. A., Vanderspurt, C., Kim, T. J., Taylor, R. K., Hillman, J. D., Progulske-Fox, A., Handfield, M., Ryan, E. T., and Calderwood, S. B. 2003. Use of in vivo-induced antigen technology (IVIAT) to identify genes uniquely expressed during human infection with Vibrio cholerae. Proc. Natl. Acad. Sci. USA 100:8508-8513.
Heithoff, D. M., Sinsheimer, R. L., Low, D. A. and Mahan M. J. 1999. An essential role for DNA adenine methylation in bacterial virulence. Science 284:967-970.
Hensel, M., Shea, J. E., Raupach, B., Monack, D., Falkow, S., Gleeson, C., Kubo, T., Holden, D. W. 1997. Functional analysis of ssaJ and the ssaK/U operon, 13 genes encoding components of the type III secretion apparatus of Salmonella pathogenicity island 1 and 2. Mol. Microbiol. 24:155-167.
Hensel, M., J. E. Shea, C. Gleeson, M. D. Jones, E. Dalton, D. W. Holden. 1995. Simultaneous identification of bacterial virulence genes by negative selection. Science 269:400-3.
Jensen, P. E. and Kapp, J. A. 1986. Bystander help in primary immune responses in vivo. J. Exptl. Med. 164:841-54.
Kato-Maeda, M., Bifani, P. J., Kreiswirth, B. N., and Small, P. M. 2001. The nature and consequence of genetic variability within Mycobacterium tuberculosis. J. Clin. Invest. 107:533-537.
Kim, Y. R., Lee, S. E., Kim, C. M., Kim, S. Y., Shin, E. K., Shin, D. H., Chung, S. S., Choy, H. E., Progulske-Fox, A., Hillman, J. D., Handfield and M., Rhee, J. H. 2003. Characterization and Pathogenic Significance of Vibrio vulnificus Antigens Preferentially Expressed in Septicemic Patients. Infect. Immun. 7:5461-5471.
Landowski, C. P., Godfrey, H. P., Bentley-Hibbert, S. I., Liu, X., Huang, Z., Sepulveda, R., Huygen, K., Gennaro, M. L., Moy, F. H., Lesley, S. A. and Haak-Frendscho, M. 2001. Combinatorial use of antibodies to secreted mycobacterial proteins in a host immune system-independent test for tuberculosis. J. Clin. Microbiol. 39:2418-2424.
Lee, S. H, S. M. Butler, and A. Camilli. 2001. Selection for in vivo regulators of bacterial virulence. Proc. Natl. Acad. Sci. USA 98:6889-94.
Lehoux, D. E., Sanschagrin, F., and Levesque, R. C. 2000. Genomics of the 35-kb pvd locus and analysis of novel pvdIJK genes implicated in pyoverdine biosynthesis in Pseudomonas aeruginosa. FEMS Microbiol. Lett. 190:141-146.
Li, Y., Frey, E., MacKenzie, A. M. R. and Finlay, B. B. 2000. Human response to Escherichia coli 0157:H7 infection: antibodies to secreted virulence factors. Infect. Immun. 68:5090-5095.
Lyashchenko, K. P., Pollock, J. M., Colangeli, R. and Gennaro, M. L. 1998a. Diversity of antigen recognition by serum antibodies in experimental bovine tuberculosis. Infect. Immun. 66:5344-5349.
Lyashchenko, K. P., Colangeli, R., Houde, M., Al-Jahdali, H., Menzies, D. and Gennaro, M. L. 1998b. Heterogeneous antibody responses in tuberculosis. Infect. Immun. 66:3936-3940.
Lyashchenko, K. P., Singh, M., Colangeli, R and Gennaro, M. L. 2000. A multi-antigen print immunoassay for the development of serological diagnosis of infectious diseases. J. Immunol. Meth. 242:91-100.
Mahan, M. J., Slauch, J. M., and Mekalanos, J. J. 1993. Selection of bacterial virulence genes that are specifically induced in host tissues. Science 259:686-688.
Manohar, J., Kudva, I. T., Griffin, R. W., Dodson, A., McManus, B., Krastins, B., Sarracino, D., Progulske-Fox, A., Hillman, J. D., Handfield, M., Tarr, P. I. and Calderwood, S. B. Identification of Escherichia coli 0157:H7 proteins expressed specifically during human infection using in vivo-induced antigen technology (IVIAT). Mol. Microbiol., In Press.
Mekalanos, J. J. 1992. Environmental signals controlling expression of virulence determinants in bacteria. J. Bacteriol. 174:1-7.
Merrell, D. S., S. M. Butler, F. Qadri, N. A. Dolganov, A. Alam, M. B. Cohen, S. B. Calderwood, G. K. Schoolnik, A. Camilli. 2002. Host-induced epidemic spread of the cholera bacterium. Nature 417:642-4.
Mollenkopf, H., Dietrich, G. and Kaufmann, S. H. 2001. Intracellular bacteria as targets and carriers for vaccination. Biol. Chem. 382:521-532.
Mustafa, A. S., Lundin, K. E. A., Meloen, R. H., Shinnick, T. M., and Oftung, F. 1999. Identification of promiscuous epitopes from the mycobacterial 65-kilodalton heat shock protein recognized by human CD4(+) T cells of the Mycobacterium leprae memory repertoire. Infect. Immun. 67:5683-5689.
Orme, I. M., McMurray, D. N. and Belisle, J. T. 2001. Tuberculosis vaccine development: recent progress. Trends Microbiol. 9:115-118.
Pelicic, V., Reyrat, J.-M., and Gicquel, B. 1998. Genetic advances for studying Mycobacterium tuberculosis pathogenicity. Molec. Microbiol. 28:413-420.
Pieters, J. 2001. Entry and survival of pathogenic mycobacteria in macrophages. Microbes Infect. 3:249-255.
Pine, R. 2002. IRF and tuberculosis. J. Interferon Cytokine Res. 22:15-25.
Schoolnik, G. K. 2002. Microarray analysis of bacterial pathogenicity. Adv. Microb. Physiol. 46:1-45.
Selinger D W, Saxena R M, Cheung K J, Church G M, Rosenow C. 2003. Global RNA half-life analysis in Escherichia coli reveals positional patterns of transcript degradation. Genome Res. 13:216-23.
Sharma, A. K. and Khuller, G. K. 2001. DNA vaccines: future strategies and relevance to intracellular pathogens. Immunol. Cell Biol. 79:537-546.
Slauch, J. M., and Camilli, A. 2000. IVET and RIVET: Use of gene fusions to identify bacterial virulence factors specifically induced in host tissues. Method. Enzymol. 326:73-96.
Smith, H. 1998. What happens to bacterial pathogens in vivo ? Trends Microbiol. 6:239-243.
Smith, D., Wiegeshaus, E. and Balasubramanian, V. 2000. Animal models for experimental tuberculosis. Clin. Infect. Dis. 31:S68-S70.
Song, Y.-H., Kozarov, E. V., Walters, S. M., Cao, S. L., Handfield, M., Hillman, J. D. and Progulske-Fox, A. 2002. Genes of periodontopathogens expressed during human disease. Ann. Periodontol 7:38-42.
Stanislavsky, E. S. and Lam, J. S. 1997. Pseudomonas aeruginosa antigens as potential vaccines. FEMS Microbiol. Rev. 21:243-277.
Tang, C. M., Bakshi, S, and Sun, Y. H. 2001. Identification of bacterial genes required for in-vivo survival. J. Pharm. Pharmacol. 53:1575-1579.
Tsuyuguchi, I. 2000. Riddles in human tuberculosis infection. Kekkaku-Tuberculosis. 75:603-609.
Valway, S. E., Sanchez, M. P., Shinnick, T. F., Orme, I., Agerton, T., Hoy, D., Jones, J. S., Westmoreland, H. and Onorato, I. M. 1998. An outbreak involving extensive transmission of a virulent strain of Mycobacterium tuberculosis. The New England J. of Med. 338:633-639.
Wallis, R. S., Phillips, M., Johnson, J. L., Teixeira, L., Rocha, L. M. M. C., Maciel, E., Rose, L., Well, C., Palaci, M., Dietze, R., Eisenach, K. and Ellner, J. J. 2001. Inhibition of isoniazid-induced expression of Mycobacterium tuberculosis antigen 85 in sputum: potential surrogate marker in tuberculosis chemotherapy trials. Antimicrob. Agents Chemother. 45:1302-1304.
Wang, J., Mushegian, A., Lory, S., and Jin, S. 1996, Large scale isolation of candidate virulence genes of Pseudomonas aeruginosa by in vivo selection. Proc. Natl. Acad. Sci. USA 93:10434-10439.
Wayne, L. G., and Sohaskey, C. D. 2001. Nonreplicating persistence of Mycobacterium tuberculosis. Annu. Rev. Microbiol. 55:139-163.
WHO. 1994. TB-a global emergency. WHO report on the TB epidemic. (ed.) Geneva.
WHO. 1997. Anti-tuberculosis drug resistance in the world. WHO/IUATLD global project on anti-tuberculosis drug resistance surveillance (ed.). Geneva.
Xu, Q., M. Dziejman, and J. J. Mekalanos. 2003. Determination of the transcriptome of Vibrio cholerae during intraintestinal growth and midexponential phase in vitro. Proc. Natl. Acad. Sci. USA 100:1286-91.
Yi, M. Y., Kaneko, S., Yu, D. Y., and Murakami, S. 1997. Hepatitis C virus envelope proteins bind lactoferrin. J. Virol. 71:5997-6002.
Zahrani, K., Al-Jahdali, H., Poirier, L., Rene, P., Gennaro, M. L., and Menzies, D. 2000. Accuracy and utility of commercially available amplification and serologic tests for the diagnosis of minimal pulmonary tuberculosis. Am. J. Respir. Crit. Care Med. 162:1323-1329.

APPENDIX 1.
Table of IVI genes of M. tuberculosis identified by IVIAT.

| Clone | ORF | Homologue/Orthologue/Function | | Protein Specs | |
|---|---|---|---|---|---|
| 388 | CDC1551: | Closest homologue | PIR: C70623 hypothetical protein Rv1024 - | Length [aa] | 228 |
| | gi13880624 | (BLASTP) | Mycobacterium tuberculosis (strain H37RV) 1e−83 | Molecular | 24569.9 |
| | SEQ ID NO: 1 | *COMP | Mycobacterium bovis AF2122: orf94 Predicted orf 5e−88 | weight [Da] | |
| | | COGs | COG2919 Septum formation initiator 2e−92 | Isoelectric | 9.8 |
| | | INTERPRO | IPR000694 Proline-rich region (1) | point | |
| | | domains | | GC content | 68.9 |
| | | | | [%] | |
| 467 | CDC1551: | Closest homologue | PIR: E70718 hypothetical protein Rv0967 - | Length [aa] | 119 |
| | gi13880561 | (BLASTP) | Mycobacterium tuberculosis (strain H37RV) 9e−58 | Molecular | 12799.7 |
| | SEQ ID NO: 2 | *COMP | Mtuberculosis CDC1551: gi_13880561 conserved | weight [Da] | |
| | | | hypothetical protein [Mycobacterium tuberculosis | Isoelectric | 6.2 |
| | | | CDC1551] 3e−59 | point | |
| | | COGs | COG1937 Uncharacterized BCR 4e−45 | GC content | 62.8 |
| | | INTERPRO | IPR003735 Protein of unknown function DUF156 (1) | [%] | |
| | | domains | | | |
| 251 | CDC1551: | Closest homologue | PIR: H70763 probable glgY protein - Mycobacterium | Length [aa] | 765 |
| | gi13881241 | (BLASTP) | tuberculosis (strain H37RV) 0.0 | Molecular | 82706.9 |
| | SEQ ID NO: 3 | *COMP | Mtuberculosis CDC1551: gi_13881241 maltooligosyl | weight [Da] | |
| | | | trehalose synthase [Mycobacterium tuberculosis | Isoelectric | 5.7 |
| | | | CDC1551] 0.0 | point | |
| | | COGs | COG3280 Maltooligosyl trehalose synthase 0.0 | GC content | 66.7 |
| | | | COG0366 Glycosidases 4e−70 | [%] | |
| | | INTERPRO | IPR000461 Glycoside hydrolase family 13 (1) | | |
| | | domains | | | |
| 192 | CDC1551: | Closest homologue | PIR: G70668 polyketide synthase pks1 - Mycobacterium | Length [aa] | 4151 |
| | gi13881781 | (BLASTP) | tuberculosis (strain H37RV) 0.0 | Molecular | 431582.0 |
| | SEQ ID NO: 4 | *COMP | Mtuberculosis CDC1551: gi_13881334 polyketide synthase | weight [Da] | |
| | | | [Mycobacterium tuberculosis CDC1551] 0.0 | Isoelectric | 5.0 |
| | | Automatically | 32 CELL RESCUE, DEFENSE AND VIRULENCE 0.0 - | point | |
| | | derived functional | N. crassa_put | GC content | 67.5 |
| | | categories | 32.05 disease, virulence and defense 0.0 - N. crassa_put | [%] | |
| | | | 32.05.05 virulence, disease factors 0.0 N. crassa_put | | |
| | | COGs | COG0304 3-oxoacyl-(acyl-carrier-protein) synthase I 1e−127 | | |
| | | INTERPRO | IPR000794 Beta-ketoacyl synthase (6) | | |
| | | domains | IPR001227 Acyl transferase (2) | | |
| | | | IPR001899 Surface protein from Gram-positive cocci, | | |
| | | | anchor region (1) | | |
| | | | IPR002085 Zinc-containing alcohol dehydrogenase | | |
| | | | superfamily (2) | | |
| | | | IPR003880 Phosphopantetheine attachment site (6) | | |
| 124 | CDC1551: | Closest homologue | PIR: A70809 probable 5′-phosphoribosyl-5- | Length [aa] | 364 |
| | gi13880377 | (BLASTP) | aminoimidazole synthetase - Mycobacterium tuberculosis | Molecular | 38393.5 |
| | SEQ ID NO: 5 | | (strain H37RV) 0.0 | weight [Da] | |
| | | *COMP | Mtuberculosis CDC1551: gi_13880377 | Isoelectric | 6.0 |
| | | | phosphoribosylformylglycinamidine cyclo-ligase | point | |
| | | | [Mycobacterium tuberculosis CDC1551] 0.0 | GC content | 65.0 |
| | | Automatically | 01.03.01.03 purine nucleotide anabolism 1e−122 - | [%] | |
| | | derived functional | L. innocua_put | | |
| | | categories | 01 METABOLISM 1e−122 - L. innocua_put | | |
| | | COGs | COG0150 Phosphoribosylaminoimidazol (AIR) synthetase | | |
| | | | 1e−104 | | |
| | | INTERPRO | IPR000728 AIR synthase related protein (2) | | |
| | | domains | | | |
| 154 | CDC1551: | Closest homologue | PIR: F70537 hypothetical protein Rv1117 - Mycobacterium | Length [aa] | 107 |
| | Gi13880733 | (BLASTP) | tuberculosis (strain H37RV) 5e−52 | Molecular | 12049.5 |
| | SEQ ID NO: 6 | *COMP | Mtuberculosis CDC1551: gi_13880733 conserved | weight [Da] | |
| | | | hypothetical protein [Mycobacterium tuberculosis | Isoelectric | 4.7 |
| | | | CDC1551] 1e−53 | point | |
| | | COGs | COG1359 Uncharacterized ACR 5e−33 | GC content | 63.0 |
| | | | COG2329 Uncharacterized enzyme of polyketide | [%] | |
| | | | biosynthesis | | |
| | | INTERPRO | IPR003771 Protein of unknown function DUF176 (1) | | |
| | | domains | | | |
| 213 | CDC1551: | Closest homologue | PIR: S72609 GTP-binding membrane protein lepA - | Length [aa] | 653 |
| | gi13882191 | (BLASTP) | Mycobacterium leprae 0.0 | Molecular | 72364.0 |
| | SEQ ID NO: 7 | *COMP | Mtuberculosis CDC1551: gi_13882191 GTP-binding | weight [Da] | |
| | | | protein [Mycobacterium tuberculosis CDC1551] 0.0 | Isoelectric | 5.6 |
| | | Automatically | 16.19.05 GTP binding 0.0 - B. subtilis_exp | point | |
| | | derived functional | 98 CLASSIFICATION NOT YET CLEAR-CUT 0.0 - | GC content | 63.9 |
| | | categories | B. subtilis_exp | [%] | |
| | | | 16 PROTEIN WITH BINDING FUNCTION OR | | |
| | | | COFACTOR REQUIREMENT (structural or catalytic) | | |
| | | | 0.0 - B. subtilis_exp | | |
| | | COGs | COG0481 Membrane GTPase LepA 0.0 | | |
| | | INTERPRO | IPR000640 Elongation factor G, C-terminal (1) | | |
| | | domains | IPR000795 Elongation factor, GTP-binding (7) | | |
| | | | IPR001687 ATP/GTP-binding site motif A (P-loop) (1) | | |
| | | | IPR004161 Elongation factor Tu, domain 2 (1) | | |
| 255 | CDC1551: | Closest homologue | PIR: H70783 hypothetical protein Rv2191 - | Length [aa] | 645 |
| | gi13881937 | (BLASTP) | Mycobacterium tuberculosis (strain H37RV) 0.0 | Molecular | 69148.1 |
| | SEQ ID NO: 8 | *COMP | Mtuberculosis CDC1551: gi_13881937 DNA polymerase | weight [Da] | |
| | | | III, epsilon subunit, putative [Mycobacterium tuberculosis | Isoelectric | 9.6 |
| | | | CDC1551] 0.0 | point | |
| | | COMPLETE COGs | COG0322 Nuclease subunit of the excinuclease complex | GC content | 69.7 |
| | | | 1e−96 | [%] | |
| | | INTERPRO | IPR000305 Excinuclease ABC, C subunit, N-terminal (3) | | |
| | | domains | IPR000520 Exonuclease (4) | | |
| 141 | CDC1551: | Closest homologue | PIR: D70531 hypothetical protein Rv2705c - | Length [aa] | 129 |
| | gi13882534 | (BLASTP) | Mycobacterium tuberculosis (strain H37RV) 6e−60 | Molecular | 14055.0 |
| | SEQ ID NO: 9 | *COMP | Mycobacterium bovis AF2122: orf26 Predicted orf 1e−61 | weight [Da] | |
| | | Automatically | 32 CELL RESCUE, DEFENSE AND VIRULENCE 4e−33 - | Isoelectric | 5.9 |
| | | derived functional | A. thaliana_put | point | |
| | | categories | 32.05 disease, virulence and defense 4e−33 - A. thaliana_put | GC content | 65.9 |
| | | | 32.05.01 resistance proteins 4e−33 - A. thaliana_put | [%] | |
| | | PFAM domains | PF06108 Protein of unknown function (DUF952) 3e−60 | | |
| | | Automatically | PIR: D70531 Mycobacterium tuberculosis hypothetical | | |
| | | derived PIR | protein Rv2705c 6e−60 | | |
| | | superfamilies | | | |
| 241-a | CDC1551: | Closest homologue | PIR: C70835 hypothetical protein Rv0277c - | Length [aa] | 142 |
| | gi13879778 | (BLASTP) | Mycobacterium tuberculosis (strain H37RV) 1e−67 | Molecular | 15887.3 |
| | SEQ ID NO: 10 | *COMP | Mtuberculosis CDC1551: gi_13879778 hypothetical | weight [Da] | |
| | | | protein [Mycobacterium tuberculosis CDC1551] 1e−68 | Isoelectric | 6.7 |
| | | COGs | COG1848 Predicted nucleic acid-binding protein, contains | point | |
| | | | PIN domain 5e−45 | GC content | 62.2 |
| | | INTERPRO | IPR002716 PilT protein, N terminal (1) | [%] | |
| | | domains | | | |
| 241-b | CDC1551: | Closest homologue | PIR: B70835 hypothetical protein Rv0276 - | Length [aa] | 306 |
| | gi13879777 | (BLASTP) | Mycobacterium tuberculosis (strain H37RV) 1e−162 | Molecular | 34779.3 |
| | SEQ ID NO: 11 | *COMP | Mtuberculosis CDC1551: gi_13879777 hypothetical | weight [Da] | |
| | | | protein [Mycobacterium tuberculosis CDC1551] 1e−163 | GC content | 66.9 |
| | | | | [%] | |
| 371-a | CDC1551: | Closest homologue | PIR: B70811 hypothetical protein Rv0826 - | Length [aa] | 351 |
| | gi13880397 | (BLASTP) | Mycobacterium tuberculosis (strain H37RV) 0.0 | Molecular | 40249.1 |
| | SEQ ID NO: 12 | *COMP | Mtuberculosis CDC1551: gi_13880397 hypothetical | weight [Da] | |
| | | | protein [Mycobacterium tuberculosis CDC1551] 0.0 | Isoelectric | 9.3 |
| | | Automatically | PIR: B70811 Mycobacterium tuberculosis hypothetical | point | |
| | | derived PIR | protein Rv1645c 0.0 | GC content | 66.2 |
| | | superfamilies | | [%] | |
| 371-b | CDC1551: | Closest homologue | PIR: C70811 hypothetical protein Rv0827c - | Length [aa] | 130 |
| | gi13880398 | (BLASTP) | Mycobacterium tuberculosis (strain H37RV) 1e−65 | Molecular | 14296.3 |
| | SEQ ID NO: 13 | *COMP | Mtuberculosis CDC1551: gi_13880398 transcriptional | weight [Da] | |
| | | | regulator, ArsR family [Mycobacterium tuberculosis | Isoelectric | 7.0 |
| | | | CDC1551] 9e−67 | point | |
| | | Automatically | 11.02.03.04 transcriptional control 3e−33 - | GC content | 64.4 |
| | | derived functional | L. monocytogenes_put | [%] | |
| | | categories | 16.03.01 DNA hinding 3e−33 - L. monocytogenes_put | | |
| | | | 32.05.01.03.03 inorganic chemical agent resistance (e.g. | | |
| | | | heavy metals) 3e−33 - L. monocytogenes_put | | |
| | | | 32 CELL RESCUE, DEFENSE AND VIRULENCE 3e−33 - | | |
| | | | L. monocytogenes_put | | |
| | | | 70 SUBCELLULAR LOCALIZATION 3e−33 - | | |
| | | | L. monocytogenes_put | | |
| | | COGs | COG0640 Predicted transcriptional regulators 5e−38 | | |
| | | INTERPRO | IPR001845 Bacterial regulatory protein ArsR (6) | | |
| | | domains | | | |
| 22 | CDC1551: | Closest homologue | TRCDSEMBL: AE007134_1 gene: “MT3167”; product: | Length [aa] | 340 |
| | gi13882970 | (BLASTP) | “transcriptional regulator, AraC family”; Mycobacterium | Molecular | 37789.4 |
| | SEQ ID NO: 14 | | tuberculosis CDC1551, section 220 of 280 of the complete | weight [Da] | |
| | | | genome. 0.0 | Isoelectric | 9.2 |
| | | *COMP | Mtuberculosis CDC1551: gi_13882970 transcriptional | point | |
| | | | regulator, AraC family [Mycobacterium tuberculosis | GC content | 66.9 |
| | | | CDC1551] 0.0 | [%] | |
| | | COGs | COG2207 AraC-type DNA-binding domain-containing | | |
| | | | proteins 4e−78 | | |
| | | INTERPRO | IPR000005 Helix-turn-helix, AraC type (4) | | |
| | | domains | | | |
| | CDC1551: | Closest | PIR: G70928 probable 4-ALPHA-GLUCANOTRANSFERASE - | Length [aa] | 724 |
| | gi13881476 | homologue | Mycobacterium tuberculosis (strain H37RV) 0.0 | Molecular | 79745.1 |
| | SEQ ID NO: 15 | (BLASTP) | | weight [Da] | |
| | | *COMP | Mtuberculosis CDC1551: gi_13881476 4-alpha- | Isoelectric | 5.4 |
| | | | glucanotransferase/amylomaltase/disproportionating enzyme | point | |
| | | | [Mycobacterium tuberculosis CDC1551] 0.0 | GC content | 68.0 |
| | | COGs | COG1640 4-alpha-glucanotransferase 0.0 | [%] | |
| | | PFAM | PF02446 4-alpha-glucanotransferase 5.5e−251 | | |
| | | domains | | | |
| | | BLOCKS | IPB003385 4-alpha-glucanotransferase | | |
| | | INTERPRO | IPR003385 Glycoside hydrolase, family 77 (1) | | |
| | | domains | | | |
| | | Automatically | PIR: G70928 4-alpha-glucanotransferase 0.0 | | |
| | | derived PIR | | | |
| | | superfamilies | | | |
| | | Automatically | PIR: AC0016 2.4.1.25 4-a-glucanotransferase 9e−77 | | |
| | | derived EC | | | |
| | | numbers | | | |
| | CDC1551: | Closest | PIR: H70647 probable NADH2 dehydrogenase (ubiquinone) | Length [aa] | 806 |
| | Gi13883049 | homologue | (EC 1.6.5.3) I chain G - Mycobacterium tuberculosis (strain | Molecular | 85392.2 |
| | SEQ ID NO: 16 | (BLASTP) | H37RV) 0.0 | weight [Da] | |
| | | *COMP | Mtuberculosis CDC1551: gi_13883049 NADH dehydrogenase | Isoelectric | 5.4 |
| | | | I, G subunit [Mycobacterium tuberculosis CDC1551] 0.0 | point | |
| | | *HUMAN | TRANSLATION: ENSP00000233190 Database: core | GC content | 68.5 |
| | | | Gene: ENSG00000023228 Clone: AC007383 | [%] | |
| | | | Contig: AC007383.4.1.214978 Chr: 2 Basepair: 206952932 | | |
| | | | Status: known 1e−62 | | |
| | | Automatically | 01.05.01.01.05 C-1 compound catabolism 0.0 - | | |
| | | derived | L. monocytogenes_put | | |
| | | functional | 02.13.01 anaerobic respiration 0.0 - L. monocytogenes_put | | |
| | | categories | 16.17.09 heavy metal binding (Cu, Fe, Zn) 0.0 - | | |
| | | | L. monocytogenes_put | | |
| | | | 16.21.03 molybdopterin binding 0.0 - L. monocytogenes_put | | |
| | | | 70.03 cytoplasm 0.0 - L. monocytogenes_put | | |
| | | | 01 METABOLISM 0.0 - L. monocytogenes_put | | |
| | | | 02 ENERGY 0.0 - L. monocytogenes_put | | |
| | | | 16 PROTEIN WITH BINDING FUNCTION OR COFACTOR | | |
| | | | REQUIREMENT (structural or catalytic) 0.0 - | | |
| | | | L. monocytogenes_put | | |
| | | | 70 SUBCELLULAR LOCALIZATION 0.0 - | | |
| | | | L. monocytogenes_put | | |
| | | | 01.05 C-compound and carbohydrate metabolism 0.0 - | | |
| | | | L. monocytogenes_put | | |
| | | | 02.13 respiration 0.0 - L. monocytogenes_put | | |
| | | | 16.17 metal binding 0.0 - L. monocytogenes_put | | |
| | | | 16.21 complex cofactor/cosubstrate binding 0.0 - | | |
| | | | L. monocytogenes_put | | |
| | | | 01.05.01 C-compound and carbohydrate utilization 0.0 - | | |
| | | | L. monocytogenes_put | | |
| 95-a* | CDC 1551: | Closest homologue | PIR: F70665 probable NADH dehydrogenase - | Length [aa] | 463 |
| | 13881553 | (BLASTP) | Mycobacterium tuberculosis (strain H37RV) 0.0 | Molecular | 49619.2 |
| | SEQ ID NO: 17 | *COMP | Mtuberculosis CDC1551: gi_13881553 NADH | weight [Da] | |
| | | | dehydrogenase [Mycobacterium tuberculosis CDC1551] | Isoelectric | 9.3 |
| | | | 0.0 | point | |
| | | Automatically | 02 ENERGY 1e−115 - A. thaliana_put | GC content | 64.1 |
| | | derived functional | 02.13 respiration 1e−115 - A. thaliana_put | [%] | |
| | | categories | | | |
| | | COGs | COG1252 NADH dehydrogenase, FAD-containing subunit | | |
| | | | 1e−105 | | |
| | | | COG1249 Dihydrolipoamide dehydrogenase/glutathione | | |
| | | | oxidoreductase and related enzymes 1e−60 | | |
| | | INTERPRO | IPR001327 FAD-dependent pyridine nucleotide-disulphide | | |
| | | domains | oxidoreductase (4) | | |
| 95-b* | CDC1551: | Closest homologue | TRCDSEMBL: AE007047_17 gene: “MT1901”; product: | Length [aa] | 208 |
| | 13881552 | (BLASTP) | “hypothetical protein”; Mycobacterium tuberculosis | Molecular | 22303.2 |
| | SEQ ID NO: 18 | | CDC1551, section 133 of 280 of the complete genome. 1e−100 | weight [Da] | |
| | | *COMP | Mtuberculosis CDC1551: gi_13881552 hypothetical | Isoelectric | 9.6 |
| | | | protein [Mycobacterium tuberculosis CDC1551] 1e−101 | point | |
| | | BLOCKS | IPB002669 UreD urease accessory protein | Manually | no |
| | | | | edited | |
| | | | | Contig name | Mtuberculosis |
| | | | | Position | 2101021-2101647 |
| | | | | GC content | 68.3 |
| | | | | [%] | |
| 134 | CDC 1551: | Closest homologue | PIR: F70502 hypothetical protein Rv1695 - Mycobacterium | Length [aa] | 307 |
| | 13881371 | (BLASTP) | tuberculosis (strain H37RV) 1e−159 | Molecular | 32871.7 |
| | SEQ ID NO: 19 | *COMP | Mtuberculosis CDC1551: gi_13881371 conserved | weight [Da] | |
| | | | hypothetical protein [Mycobacterium tuberculosis | Isoelectric | 5.3 |
| | | | CDC1551] 1e−160 | point | |
| | | Automatically | 01.03 nucleotide metabolism 4e−76 - H. pylori_put | GC content | 64.9 |
| | | derived functional | 01.07.01 biosynthesis of vitamins, cofactors, and prosthetic | [%] | |
| | | categories | groups 2e−75 - B. subtilis_put | | |
| | | COGs | COG0061 Predicted kinase 1e−112 | | |
| | | INTERPRO | IPR002504 ATP-NAD kinase (1) | | |
| | | domains | | | |
| 373 | CDC 1551: | Closest homologue | PIR: A87112 heat shock protein Hsp90 family [imported] - | Length [aa] | 647 |
| | 13882052 | (BLASTP) | Mycobacterium leprae 0.0 | Molecular | 72961.2 |
| | SEQ ID NO: 20 | *COMP | Mtuberculosis CDC1551: gi_13882052 heat shock protein | weight [Da] | |
| | | | HtpG [Mycobacterium tuberculosis CDC1551] 0.0 | Isoelectric | 4.8 |
| | | Automatically | 14.01 protein folding and stabilization 0.0 - A. thaliana_put | point | |
| | | derived functional | 70.03 cytoplasm 0.0 - A. thaliana_put | Manually | no |
| | | categories | 70.04.05 microtubule cytoskeleton 0.0 - A. thaliana_put | edited | |
| | | COGs | COG0326 Molecular chaperone, HSP90 family 0.0 | Contig name | Mtuberculosis |
| | | | COG0187 DNA gyrase (topoisomerase II) B subunit 4e−52 | Position | 2572001-2570058 |
| | | INTERPRO | IPR001404 Heat shock protein Hsp90 (9) | GC content | 62.0 |
| | | domains | IPR003594 ATP-binding protein, ATPase-like (2) | [%] | |
| 85 | CDC 1551: | Closest homologue | PIR: F87206 conserved large membrane protein ML2378 | Length [aa] | 958 |
| | 13879914 | (BLASTP) | [imported] - Mycobacterium leprae 0.0 | Molecular | 104807.0 |
| | SEQ ID NO: 21 | *COMP | Mtuberculosis CDC1551: gi_13879914 membrane protein, | weight [Da] | |
| | | | MmpL family [Mycobacterium tuberculosis CDC1551] 0.0 | Isoelectric | 6.7 |
| | | Automatically | 70.30 prokaryotic cytoplasmic membrane 0.0 - | point | |
| | | derived functional | B. subtilis_put | GC content | 60.5 |
| | | categories | 70 SUBCELLULAR LOCALIZATION 0.0 - | [%] | |
| | | | B. subtilis_put | | |
| | | COGs | COG2409 Predicted transporters 0.0 | | |
| | | | COG0841 Cation/multidrug efflux pump 1e−107 | | |
| | | INTERPRO | IPR004707 Transport protein (1) | | |
| | | domains | IPR004869 MMPL domain (2) | | |
| 92 | CDC 1551: | Closest homologue | TRCDSEMBL: AE006925_4 gene: “MT0132”; product: | Length [aa] | 487 |
| | 13879614 | (BLASTP) | “PE_PGRS family protein”; Mycobacterium tuberculosis | Molecular | 40891.1 |
| | SEQ ID NO: 22 | | CDC1551, section 11 of 280 of the complete genome. 6e−29 | weight [Da] | |
| | | *COMP | Mtuberculosis CDC1551: gi_13879614 PE_PGRS family | | |
| | | | protein [Mycobacterium tuberculosis CDC1551] 2e−30 | Isoelectric | 4.9 |
| | | INTERPRO | IPR000084 PE N-terminal (1) | point | |
| | | domains | IPR002173 Carbohydrate kinase, PfkB (3) | GC content | 77.7 |
| | | | | [%] | |
| 115 | CDC 1551: | Closest homologue | PIR: C70815 probable beta-ketoadipyl CoA thiolase - | Length [aa] | 403 |
| | 13880432 | (BLASTP) | Mycobacterium tuberculosis (strain H37RV) 0.0 | Molecular | 42414.7 |
| | SEQ ID NO: 23 | *COMP | Mycobacterium bovis AF2122: orf27 Predicted orf 0.0 | weight [Da] | |
| | | Automatically | 02 ENERGY 1e−128 - A. thaliana_put | Isoelectric | 5.2 |
| | | derived functional | 70.16 mitochondrion 1e−128 - A. thaliana_put | point | |
| | | categories | 01.05 C-compound and carbohydrate metabolism 1e−122 - | GC content | 67.1 |
| | | | B. subtilis_put | [%] | |
| | | COGs | COG0183 Acetyl-CoA acetyltransferases 1e−118 | | |
| | | INTERPRO | IPR002155 Thiolase (5) | | |
| | | domains | | | |
| 126 | CDC 1551: | Closest homologue | TRCDSEMBL: AE006971_7 gene: “MT0807”; product: | Length [aa] | 540 |
| | 13880353 | (BLASTP) | “drug transporter”; Mycobacterium tuberculosis | Molecular | 56587.4 |
| | SEQ ID NO: 24 | | CDC1551, section 57 of 280 of the complete genome. 0.0 | weight [Da] | |
| | | *COMP | Mtuberculosis CDC1551: gi_13880353 drug transporter | Isoelectric | 10.1 |
| | | | [Mycobacterium tuberculosis CDC1551] 0.0 | point | |
| | | Automatically | 20.01.27 drug transport 1e−46 - B. subtilis_put | GC content | 63.6 |
| | | derived functional | 20.03.25 ABC transporters 1e−46 - B. subtilis_put | [%] | |
| | | categories | 70 SUBCELLULAR LOCALIZATION 1e−46 - | | |
| | | | B. subtilis_put | | |
| | | COGs | COG0477 Permeases of the major facilitator superfamily | | |
| | | | 4e−66 | | |
| | | INTERPRO | IPR003662 General substrate transporter (1) | | |
| | | domains | IPR004638 Drug resistance transporter EmrB/QacA | | |
| | | | subfamily (1) | | |
| 210 | CDC 1551: | Closest homologue | PIR: C70668 probable mmpL7 protein - Mycobacterium | Length [aa] | 920 |
| | 13882795 | (BLASTP) | tuberculosis (strain H37RV) 0.0 | Molecular | 95122.1 |
| | SEQ ID NO: 25 | *COMP | Mtuberculosis CDC1551: gi_13882795 membrane protein, | weight [Da] | |
| | | | MmpL family [Mycobacterium tuberculosis CDC1551] 0.0 | Isoelectric | 9.0 |
| | | COGs | COG2409 Predicted transporters 0.0 | point | |
| | | INTERPRO | IPR000169 Eukaryotic thiol (cysteine) protease, active site | GC content | 66.0 |
| | | domains | (1) | [%] | |
| | | | IPR001117 Multicopper oxidase, type 1 (1) | | |
| | | | IPR004869 MMPL domain (1) | | |
| 223 | CDC 1551: | Closest homologue | PIR: F70907 hypothetical protein Rv0587 - Mycobacterium | Length [aa] | 265 |
| | 13880131 | (BLASTP) | tuberculosis (strain H37RV) 1e−113 | Molecular | 27327.6 |
| | SEQ ID NO: 26 | *COMP | Mtuberculosis CDC1551: gi_13880131 conserved | weight [Da] | |
| | | | hypothetical protein [Mycobacterium tuberculosis | Isoelectric | 7.7 |
| | | | CDC1551] 1e−115 | point | |
| | | COGs | COG0767 Permease component of an ABC-transporter | GC content | 64.5 |
| | | | 2e−74 | [%] | |
| | | INTERPRO | IPR003453 Protein of unknown function DUF140 (1) | | |
| | | domains | | | |
| 242 | CDC 1551: | Closest homologue | PIRw: C70879 probable ftsK - Mycobacterium | Length [aa] | 883 |
| | 13882582 | (BLASTP) | tuberculosis (strain H37RV) 0.0 | Molecular | 94406.6 |
| | SEQ ID NO: 27 | *COMP | Mtuberculosis CDC1551: gi_13882582 cell division protein | weight [Da] | |
| | | | FtsK [Mycobacterium tuberculosis CDC1551] 0.0 | Isoelectric | 8.6 |
| | | Automatically | 10.03.03 cytokinesis (cell division)/septum formation 0.0 - | point | |
| | | derived functional | L. monocytogenes_put | GC content | 67.7 |
| | | categories | 10.03.04.05 chromosome segregation/division 0.0 - | [%] | |
| | | | L. monocytogenes_put | | |
| | | COGs | COG1674 DNA segregation ATPase FtsK/SpoIIIE and | | |
| | | | related proteins 0.0 | | |
| | | INTERPRO | IPR001687 ATP/GTP-binding site motif A (P-loop) (1) | | |
| | | domains | IPR002543 Cell divisionFtsK/SpoIIIE protein (1) | | |
| 247-a | CDC 1551: | Closest homologue | PIR: E70501 probable transmrembrane protein - | Length [aa] | 226 |
| | 13881362 | (BLASTP) | Mycobacterium tuberculosis (strain H37RV) 6e−95 | Molecular | 24392.5 |
| | SEQ ID NO: 28 | *COMP | Mycobacterium bovis AF2122: orf23 Predicted orf 1e−104 | weight [Da] | |
| | | COGs | COG0842 Permease component of an ABC-transporter | Isoelectric | 8.5 |
| | | | 4e−40 | point | |
| | | | COG1123 ATPase components of uncharacterized ABC- | GC content | 63.4 |
| | | | type transport system, contain duplicated ATPase domain | [%] | |
| | | | 2e−21 | | |
| | | INTERPRO | IPR004377 ABC transporter, DrrB efflux protein (1) | | |
| | | domains | | | |
| 247-b | CDC 1551: | Closest homologue | PIR: G70763 probable maltooligosyltrehalose | Length [aa] | 580 |
| | 13881240 | (BLASTP) | trehalohydrolase - Mycobacterium tuberculosis (strain | Molecular | 64076.8 |
| | SEQ ID NO: 29 | | H37RV) 0.0 | weight [Da] | |
| | | *COMP | Mtuberculosis CDC1551: gi_13881240 maltooligosyl | Isoelectric | 5.5 |
| | | | trehalose trehalohydrolase [Mycobacterium tuberculosis | point | |
| | | | CDC1551] 0.0 | GC content | 65.2 |
| | | Automatically | 01.05.01.01.01 sugar, glucoside, polyol and carboxylate | [%] | |
| | | derived functional | catabolism 1e−115 - L. monocytogenes_put | | |
| | | categories | 02.19 metabolism of energy reserves (e.g. glycogen, | | |
| | | | trehalose) 1e−115 - L. monocytogenes_put | | |
| | | COGs | COG0296 1,4-alpha-glucan branching enzyme 1e−160 | | |
| | | | COG0366 Glycosidases 1e−115 | | |
| | | | COG1523 Pullulanase and related glycosidases 1e−100 | | |
| | | INTERPRO | IPR000461 Glycoside hydrolase family 13 (1) | | |
| | | domains | IPR004193 Glycoside hydrolase, family 13, N-terminal (1) | | |
| 284 | CDC 1551: | Closest homologue | TRCDSEMBL: AE007036_10 gene: “MT1745”; product: | Length [aa] | 385 |
| | 13881383 | (BLASTP) | “PPE family protein”; Mycobacterium tuberculosis | Molecular | 38443.6 |
| | SEQ ID NO: 30 | | CDC1551, section 122 of 280 of the complete genome. 1e−132 | weight [Da] | |
| | | *COMP | Mtuberculosis CDC1551: gi_13881383 PPE family protein | Isoelectric | 5.0 |
| | | | [Mycobacterium tuberculosis CDC1551] 1e−134 | point | |
| | | PFAM domains | PF00823 PPE family 6e−83 | Manually | no |
| | | INTERPRO | IPR000030 Mycobacterial PPE protein (2) | edited | |
| | | domains | | Contig name | Mtuberculosis |
| | | | | Position | 1932653-1931496 |
| | | | | GC content | 67.6 |
| | | | | [%] | |
| 325 | CDC 1551: | Closest homologue | PIR: B70515 hypothetical protein Rv1877 - | Length [aa] | 687 |
| | 13881580 | (BLASTP) | Mycobacterium tuberculosis (strain H37RV) 0.0 | Molecular | 72354.2 |
| | SEQ ID NO: 31 | *COMP | Mtuberculosis CDC1551: gi_13881580 drug transporter | weight [Da] | |
| | | | [Mycobacterium tuberculosis CDC1551] 0.0 | Isoelectric | 6.3 |
| | | Automatically | 32 CELL RESCUE, DEFENSE AND VIRULENCE 5e−65 - | point | |
| | | derived functional | N. crassa_put | GC content | 64.4 |
| | | categories | 32.05 disease, virulence and defense 5e−65 - N. crassa_put | [%] | |
| | | | 32.05.01 resistance proteins 5e−65 - N. crassa_put | | |
| | | | 20.09.16 cellular export and secretion 3e−63 - N. crassa_put | | |
| | | | 32.07 detoxification 3e−63 - N. crassa_put | | |
| | | | 20.01.27 drug transport 3e−63 - N. crassa_put | | |
| | | COGs | COG0477 Permeases of the major facilitator superfamily | | |
| | | | 1e−171 | | |
| | | INTERPRO | IPR003662 General substrate transporter (2) | | |
| | | domains | | | |
| 341 | CDC 1551: | Closest homologue | PIR: B70945 hypothetical protein Rv2051c - | Length [aa] | 874 |
| | 13881785 | (BLASTP) | Mycobacterium tuberculosis (strain H37RV) 0.0 | Molecular | 93792.7 |
| | SEQ ID NO: 32 | *COMP | Mtuberculosis CDC1551: gi_13881785 apolipoprotein n- | weight [Da] | |
| | | | acyltransferase Lnt/dolichol-phosphate-mannosyl | Isoelectric | 8.4 |
| | | | transferase Dpm1 [Mycobacterium tuberculosis | point | |
| | | | CDC1551] 0.0 | Manually | no |
| | | Automatically | 01.05.01 C-compound and carbohydrate utilization 9e−53 - | edited | |
| | | derived functional | N. crassa_put | Contig name | Mtuberculosis |
| | | categories | 70 SUBCELLULAR LOCALIZATION 9e−53 - | Position | 2310754-2308130 |
| | | | N. crassa_put | GC content | 67.0 |
| | | COGs | COG0815 Apolipoprotein N-acyltransferase 0.0 | [%] | |
| | | | COG0463 Glycosyltransferases involved in cell wall | | |
| | | | biogenesis 4e−69 | | |
| | | | COG0388 Predicted amidohydrolase 6e−55 | | |
| | | INTERPRO | IPR001173 Glycosyl transferase, family 2 (2) | | |
| | | domains | IPR003010 Nitrilase/cyanide hydratase (2) | | |
| 367 | CDC 1551: | Closest homologue | PIR: B70612 probable recD protein - Mycobacterium | Length [aa] | 575 |
| | 13880175 | (BLASTP) | tuberculosis (strain H37RV) 0.0 | Molecular | 61714.8 |
| | SEQ ID NO: 33 | *COMP | Mycobacterium bovis AF2122: orf9 Predicted orf 0.0 | weight [Da] | |
| | | COGs | COG0507 ATP-dependent exoDNAse (exonuclease V), | Isoelectric | 6.7 |
| | | | alpha subunit - helicase superfamily I member 1e−176 | point | |
| | | | COG1112 Superfamily I DNA and RNA helicases and | Manually | no |
| | | | helicase subunits 9e−50 | edited | |
| | | PROSITE motifs | PROSITE: ATP GTP A | Contig name | Mtuberculosis |
| | | INTERPRO | IPR001687 ATP/GTP-binding site motif A (P-loop) (1) | Position | 721731-720004 |
| | | domains | IPR004587 Exodeoxyribonuclease V alpha suhunit (1) | GC content | 70.0 |
| | | | | [%] | |
| 370 | CDC 1551: | Closest homologue | PIR: T45096 probable arabinosyltransferase embB | Length [aa] | 1098 |
| | 13883784 | (BLASTP) | [imported] - Mycobacterium smegmatis 0.0 | Molecular | 118022.0 |
| | SEQ ID NO: 34 | *COMP | Mtuberculosis CDC1551: gi_13883784 arabinosyl | weight [Da] | |
| | | | transferase [Mycobacterium tuberculosis CDC1551] 0.0 | Isoelectric | 9.6 |
| | | PFAM domains | PF04602 Mycobacterial cell wall arabinan synthesis | point | |
| | | | protein 0 | Manually | no |
| | | | | edited | |
| | | | | Contig name | Mtuberculosis |
| | | | | Position | 4246512-4249808 |
| | | | | GC content | 67.2 |
| | | | | [%] | |
| 374 | CDC 1551: | Closest homologue | PIR: B70797 probable transferase - Mycobacterium | Length [aa] | 776 |
| | 13883713 | (BLASTP) | tuberculosis (strain H37RV) 0.0 | Molecular | 84129.3 |
| | SEQ ID NO: 35 | *COMP | Mtuberculosis CDC1551: gi_13883713 moaA/nifB/pqqE | weight [Da] | |
| | | | family protein [Mycobacterium tuberculosis CDC1551] | Isoelectric | 5.6 |
| | | | 0.0 | point | |
| | | Automatically | 01.07.01 biosynthesis of vitamins, cofactors, and prosthetic | Manually | no |
| | | derived functional | groups 8e−28 - B. subtilis_put | edited | |
| | | categories | 01.20.15.03 biosynthesis of ubiquinone 8e−28 - | Contig name | Mtuberculosis |
| | | | B. subtilis_put | Position | 4178283-4180613 |
| | | COGs | COG1964 Predicted Fe—S oxidoreductases 1e−161 | GC content | 65.1 |
| | | | COG0500 SAM-dependent methyltransferases 2e−61 | [%] | |
| | | | COG2230 Cyclopropane fatty acid synthase and related | | |
| | | | methyltransferases 4e−58 | | |
| | | | COG2100 Predicted Fe—S oxidoreductase 4e−48 | | |
| | | | COG2896 Molybdenum cofactor biosynthesis enzyme 3e−45 | | |
| | | INTERPRO | IPR000051 SAM (and some other nucleotide) binding | | |
| | | domains | motif (1) | | |
| | | | IPR000385 MoaA/nifB/pqqE family (1) | | |
| 378-a | CDC 1551: | Closest homologue | PIR: E70704 hypothetical protein Rv2324 - | Length [aa] | 85 |
| | 13882114 | (BLASTP) | Mycobacterium tuberculosis (strain H37RV) 1e−71 | Molecular | 9187.6 |
| | SEQ ID NO: 36 | *COMP | Mtuberculosis CDC1551: gi_13882090 transcriptional | weight [Da] | |
| | | | regulator, AsnC family [Mycobacterium tuberculosis | Isoelectric | 10.9 |
| | | | CDC1551] 6e−73 | point | |
| | | Automatically | 01.01.13 regulation of amino acid metabolism 1e−38 - | Manually | no |
| | | derived functional | B. subtilis_exp | edited | |
| | | categories | 11.02.03.04 transcriptional control 1e−38 - B. subtilis_exp | Contig name | Mtuberculosis |
| | | COGs | COG1522 Transcriptional regulators 7e−38 | Position | 2620271-2620528 |
| | | INTERPRO | IPR000485 Bacterial regulatory proteins, AsnC family (5) | GC content | 65.9 |
| | | domains | | [%] | |
| 378-b | CDC 1551: | Closest homologue | PIR: F70661 probable lipoprotein - Mycobacterium | Length [aa] | 139 |
| | 138882113 | (BLASTP) | tuberculosis (strain H37RV) 4e−64 | Molecular | 14140.9 |
| | SEQ ID NO: 37 | *COMP | Mtuberculosis CDC1551: gi_13882113 lipoprotein, | weight [Da] | |
| | | | putative [Mycobacterium tuberculosis CDC1551] 3e−65 | Isoelectric | 5.2 |
| | | | | point | |
| | | | | Manually | no |
| | | | | edited | |
| | | | | Contig name | Mtuberculosis |
| | | | | Position | 2619596-2620015 |
| | | | | GC content | 64.5 |
| | | | | [%] | |
| *382-a | CDC 1551: | Closest homologue | PIR: H70824 hypothetical protein Rv0749 - | Length [aa] | 142 |
| | 13880315 | (BLASTP) | Mycobacterium tuberculosis (strain H37RV) 2e−66 | Molecular | 15831.3 |
| | SEQ ID NO: 38 | *COMP | Mtuberculosis CDC1551: gi_13880315 hypothetical | weight [Da] | |
| | | | protein [Mycobacterium tuberculosis CDC1551] 1e−67 | Isoelectric | 7.1 |
| | | COGs | COG1848 Predicted nucleic acid-binding protein, contains | point | |
| | | | PIN domain 4e−45 | Manually | no |
| | | INTERPRO | IPR002716 PilT protein, N terminal (1) | edited | |
| | | domains | | Contig name | Mtuberculosis |
| | | | | Position | 841227-841655 |
| | | | | GC content | 64.1 |
| | | | | [%] | |
| *382-b | CDC 1551: | Closest homologue | TRCDSEMBL: AE006969_4 gene: “MT0774”; product: | Length [aa] | 81 |
| | 13880317 | (BLASTP) | “hypothetical protein”; Mycobacterium tuberculosis | Molecular | 8686.9 |
| | | | CDC1551, section 55 of 280 of the complete genome. 3e−40 | weight [Da] | |
| | SEQ ID NO: 39 | *COMP | Mtuberculosis CDC1551: gi_13880317 hypothetical | Isoelectric | 4.4 |
| | | | protein [Mycobacterium tuberculosis CDC1551] 1e−41 | point | |
| | | | | Manually | no |
| | | | | edited | |
| | | | | Contig name | Mtuberculosis |
| | | | | Position | 842032-842277 |
| | | | | GC content | 65.4 |
| | | | | [%] | |
| 379 | CDC 1551: | Closest homologue | PIR: D70813 probable NarL - Mycobacterium tuberculosis | Length [aa] | 216 |
| | 13880417 | (BLASTP) | (strain H37RV) 1e−102 | Molecular | 22916.4 |
| | SEQ ID NO: 40 | *COMP | Mtuberculosis CDC1551: gi_13880417 DNA-binding | weight [Da] | |
| | | | response regulator, LuxR family [Mycobacterium | Isoelectric | 6.1 |
| | | | tuberculosis CDC1551] 1e−103 | point | |
| | | Automatically | 10.01.01.01 bacterial competence 8e−54 - B. subtilis_exp | Manually | no |
| | | derived functional | 11.02.03.04 transcriptional control 8e−54 - B. subtilis_exp | edited | |
| | | categories | 16 PROTEIN WITH BINDING FUNCTION OR | Contig name | Mtuberculosis |
| | | | COFACTOR REQUIREMENT (structural or catalytic) | Position | 941105-940455 |
| | | | 8e−54 - B. subtilis_exp | GC content | 67.7 |
| | | | 30 CELLULAR COMMUNICATION/SIGNAL | [%] | |
| | | | TRANSDUCTION MECHANISM 8e−54 - B. subtilis_exp | | |
| | | | 34 INTERACTION WITH THE CELLULAR | | |
| | | | ENVIRONMENT 8e−54 - B. subtilis_exp | | |
| | | COGs | COG2197 Response regulators consisting of a CheY-like | | |
| | | | receiver domain and a HTH DNA-binding domain 4e−45 | | |
| | | | COG0745 Response regulators consisting of a CheY-like | | |
| | | | receiver domain and a HTH DNA-binding domain 1e−44 | | |
| | | PFAM domains | PF00196 Bacterial regulatory proteins, luxR family 9e−16 | | |
| | | INTERPRO | IPR000792 Bacterial regulatory protein, LuxR family (7) | | |
| | | domains | IPR001789 Response regulator receiver (4) | | |
| 441 | CDC 1551: | Closest homologue | PIR: C70572 hypothetical protein Rv2620c - | Length [aa] | 141 |
| | 13882446 | (BLASTP) | Mycobacterium tuberculosis (strain H37RV) 2e−42 | Molecular | 14638.8 |
| | SEQ ID NO: 41 | *COMP | Mycobacterium bovis AF2122: orf6 Predicted orf 5e−45 | weight [Da] | |
| | | | | Isoelectric | 11.8 |
| | | | | point | |
| | | | | Manually | no |
| | | | | edited | |
| | | | | Contig name | Mtubercu- |
| | | | | | losis_CDC |
| | | | | | 1551 |
| | | | | Position | 2944030-2943605 |
| | | | | GC content | 64.1 |
| | | | | [%] | |
| 495 | CDC 1551: | Closest homologue | PIR: C70558 probable ABC transporter cydD - | Length [aa] | 527 |
| | 13881289 | (BLASTP) | Mycobacterium tuberculosis (strain H37RV) 0.0 | Molecular | 54798.4 |
| | SEQ ID NO: 42 | *COMP | Mtuberculosis CDC1551: gi_13881289 ABC transporter, | weight [Da] | |
| | | | ATP-binding protein CydD [Mycobacterium tuberculosis | Isoelectric | 10.9 |
| | | | CDC1551] 0.0 | point | |
| | | COGs | COG1132 ABC-type multidrug/protein/lipid transport | Manually | no |
| | | | system, ATPase component 1e−109 | edited | |
| | | | COG2274 ABC-type bacteriocin/lantibiotic exporters, | Contig name | Mtubercu- |
| | | | contain an N-terminal double-glycine peptidase domain | | losis_CDC |
| | | | | | 1551 |
| | | | 1e−102 | Position | 1814180-1812597 |
| | | PFAM domains | PF00005 ABC transporter 9.1e−4 | GC content | 68.6 |
| | | INTERPRO | IPR001140 ABC transporter, transmembrane region (1) | [%] | |
| | | domains | IPR001687 ATP/GTP-binding site motif A (P-loop) (2) | | |
| | | | IPR003439 ABC transporter (4) | | |
| | | | IPR003593 AAA ATPase (1) | | |
| | CDC 1551: | Closest | PIR: B70963 hypothetical protein Rv0236c - Mycobacterium | Length [aa] | 1400 |
| | 13879733 | homologue | tuberculosis (strain H37RV) 0.0 | Molecular | 146248.0 |
| | | (BLASTP) | | weight [Da] | |
| | SEQ ID NO: 43 | *COMP | Mtuberculosis CDC1551: gi_13879733 conserved hypothetical | Isoelectric | 9.6 |
| | | | protein [Mycobacterium tuberculosis CDC1551] 0.0 | point | |
| | | Automatically | PIR: B70963 Mycobacterium leprae probable integral | GC content | 71.4 |
| | | derived PIR | membrane protein 0.0 | [%] | |
| | | superfamilies | | | |
| | CDC 1551: | Closest | PIR: E70862 hypothetical protein Rv2257c - Mycobacterium | Length [aa] | 272 |
| | 13882008 | homologue | tuberculosis (strain H37RV) 1e−131 | Molecular | 28385.1 |
| | | (BLASTP) | | weight [Da] | |
| | SEQ ID NO: 44 | *COMP | Mtuberculosis CDC1551: gi_13882008 hypothetical protein | Isoelectric | 5.2 |
| | | | [Mycobacterium tuberculosis CDC1551] 1e−132 | point | |
| | | COGs | COG1680 Beta-lactamase class C and other penicillin binding | | |
| | | | proteins 4e−79 | | |

*a, b represent 2 open reading frames on the same cloned insert, either or both of which may be responsible for expressing a protein reactive with the adsorbed serum. Subcloning will distinguish which of these are producing the desired in vivo induced protein.

1. A method for detecting an antibody specific for Mycobacterium tuberculosis in a test sample comprising contacting the test sample with a purified polypeptide comprising SEQ ID NOs:1-44 or a combination thereof and detecting formation of an immunocomplex comprising the polypeptide of SEQ ID NO:1-44 and the antibody specific for M. tuberculosis, wherein detection of the immunocomplex indicates the presence of an antibody specific for M. tuberculosis in the test sample.

2. The method of claim 1, wherein the test sample is blood, sputum, serum, or lung lavage fluid.

3. The method of claim 1, wherein the polypeptide is immobilized on a substrate.

4. The method of to claim 1, wherein the method comprises an assay selected from the group consisting of a radioimmunoassay, horizontal flow chromatography, a dot blot assay, a competitive-binding assay, a western blot, an enzyme-linked immunosorbent assay (ELISA), and a sandwich assay.

5. (canceled)

6. (canceled)

7. A method of detecting the presence or absence of a M. tuberculosis antigen in a test sample comprising contacting the test sample with the antibody or the antigen-binding portion thereof of claim 11, and detecting an immunocomplex comprising the M. tuberculosis antigen and the antibody or antigen-binding portion thereof, wherein detection of the immunocomplex indicates the presence of the M. tuberculosis antigen in the test sample.

8. The method of claim 7, wherein the test sample is blood, sputum, serum, or lung lavage fluid.

9. The method of claim 7, wherein the antibody or antigen-binding portion thereof is immobilized on a substrate.

10. The method of claim 7, wherein the method comprises an assay selected from the group consisting of a radioimmunoassay, horizontal flow chromatography, a dot blot assay a competitive-binding assay, a western blot, an ELISA, and a sandwich assay.

11. A purified antibody or antigen-binding portion thereof that binds to a polypeptide consisting of SEQ ID NOs:1-44 with a binding affinity of about Kₐ of 10⁷ l/mol or more.

12. A composition comprising the purified antibody or antigen-binding portion thereof of claim 11 and a pharmaceutically acceptable carrier.

13. (canceled)

14. (canceled)

15. A method of passively immunizing or ameliorating one or more symptoms of tuberculosis comprising administering an antibody of claim 12 to a tuberculosis patient.

16. An immunogenic composition comprising one or more purified polypeptides comprising SEQ ID NO:1-44 and one or more pharmaceutically acceptable carriers.

17. The immunogenic composition of claim 16, further comprising one or more adjuvants or immunostimulatory compounds.

18. A method of immunizing a mammal against a Mycobacterium tuberculosis infection, stimulating a Mycobacterium tuberculosis specific immunogenic response in a mammal, or reducing the severity of a Mycobacterium tuberculosis infection comprising administering the immunogenic composition of claim 16 to the mammal.

19. A fusion protein comprising one or more of polypeptides comprising SEQ ID NO:1-44 and a heterologous protein, wherein the heterologous protein can be a polypeptide comprising SEQ ID NOs:1-44.

20. The fusion protein of claim 19 wherein the heterologous protein is Mycobacterium tuberculosis Antigen 85b, Mycobacterium tuberculosis ESAT-6, Mycobacterium tuberculosis MtB41, or Mycobacterium tuberculosis Mtb39.

21. (canceled)

22. (canceled)

23. (canceled)

24. (canceled)
